SEQUENCE LISTING (16 sequences)

SEQ ID NO: 1 - protein, 118 aa, Artificial Sequence
  Artificial sequence derived from Homo sapiens sequence
GVQLLESGGGLVQPGGSLRLSCAASGFTFAWYDMGWVRQAPGKGLEWVSSIDWHGEVTYYADSVKGRFTISRDNSKNTLYLQMNSLRAEDTAVYYCATAEDEPGYDYWGQGTLVTVSS

SEQ ID NO: 2 - DNA, 354 bp, Artificial Sequence
  Artificial sequence derived from Homo sapiens sequence
ggggtgcagctgttggagtctgggggaggcttggtacagcctggggggtccctgcgtctctcctgtgcagcctccggattcaccttcgcttggtatgatatggggtgggtccgccaggctccagggaagggtctagagtgggtctcaagtattgattggcatggtgaggttacatactacgcagactccgtgaagggccggttcaccatctcccgcgacaattccaagaacacgctgtatctgcaaatgaacagcctgcgtgccgaggacaccgcggtatattactgtgcgacagcggaggacgagccggggtatgactactggggccagggaaccctggtcaccgtctctagc

SEQ ID NO: 3 - DNA, 360 bp, Artificial Sequence
  Artificial sequence derived from Homo sapiens sequence
ggtgttcaattgttggaatccggtggtggattggttcaacctggtggttctttgagattgtcctgtgctgcttccggttttactttcgcttggtacgacatgggatgggtcagacaagctcctggaaagggattggaatgggtttcctccattgattggcacggtgaagttacttactacgctgactccgttaagggaagattcactatctccagagacaactccaagaacactttgtacttgcagatgaactccttgagagctgaggatactgctgtttactactgtgctacagctgaagatgaaccaggttacgactactggggacagggaactttggttactgtttcctcctagtag

SEQ ID NO: 4 - DNA, 360 bp, Artificial Sequence
  Artificial sequence derived from Homo sapiens sequence
ggtgttcaattgttggaatccggtggtggattggttcaacctggtggttctttgagattgtcctgtgctgcttccggttttactttcgcttggtacgacatgggatgggttagacaagctcctggaaagggattggagtgggtttcctccattgattggcacggtgaagttacttactacgctgactccgttaagggaagattcactatctccagagacaactccaagaacactttgtacttgcagatgaactccttgagagctgaggatactgctgtttactactgtgctactgctgaagatgaaccaggttacgactactggggacagggaactttggttactgtttcctcctagtag

SEQ ID NO: 5 - DNA, 354 bp, Artificial Sequence
  Artificial sequence derived from Homo sapiens sequence
gaggttcaactgctggagtctggtggtggtctggttcagcctggtggtagcctgcgtctgtcttgcgtggcgtccggcttcactttcgatgttgctgagatggactgggttcgtcaggcgcctggtaaaggtctggagtgggtgtctaccattagcccatctcgtcgtggtacgtattacgctgacagcgtaaaaggtcgttttaccatctcccgcgataactctaaaaacactctgtacctgcaaatgaattctctgcgtgctgaggacaccgcagtatactactgcgcaaaagcctacactggccgttccctgtggggcccgggtaccctggtgactgtaagctcttaatga

SEQ ID NO: 6 - protein, 116 aa, Artificial Sequence
  Artificial sequence derived from Homo sapiens sequence
EVQLLESGGGLVQPGGSLRLSCVASGFTFDVAEMDWVRQAPGKGLEWVSTISPSRRGTYYADSVKGRFTISRDNSKNTLYLQMNSLRAEDTAVYYCAKAYTGRSLWGPGTLVTVSS

SEQ ID NO: 7 - protein, 116 aa, Artificial Sequence
  Artificial sequence derived from Homo sapiens sequence
EVQLLESGGGLVQPGGSLRLSCAASGFTFDVAEMDWVRQAPGKGLEWVSTISPSRRGTYYADSVKGRFTISRDNSKNTLYLQMNSLRAEDTAVYYCAKAYTGRSWWGPGTLVTVSS

SEQ ID NO: 8 - protein, 116 aa, Artificial Sequence
  Artificial sequence derived from Homo sapiens sequence
EVHLLESGGGLVQPGGSLRLSCAASGFTFDSAEMDWVRQAPGKGLEWVSTISPSRRGTYYADSVKGRFTISRDNSKNTLYLQMNSLRAEDTAIYYCAKAYTGRSYWGPGTLVTVSS

SEQ ID NO: 9 - protein, 116 aa, Artificial Sequence
  Artificial sequence derived from Homo sapiens sequence
GVQLLESGGGLVQPGGSLRLSCAASGFTFDSAEMDWVRQAPGKGLEWVSTISPSRRGTYYADSVKGRFTISRDNSKNTLYLQMNSLRAEDTAVYYCAKAYTGRSYWGPGTLVTVSS

SEQ ID NO: 10 - protein, 116 aa, Artificial Sequence
  Artificial sequence derived from Homo sapiens sequence
GVQLLESGGGLVQPGGSLRLSCVASGFTFDVAEMDWVRQAPGKGLEWVSTISPSRRGTYYADSVKGRFTISRDNSKNTLYLQMNSLRAEDTAVYYCAKAYTGRSLWGPGTLVTVSS

SEQ ID NO: 11 - DNA, 348 bp, Artificial Sequence
  Artificial sequence derived from Homo sapiens sequence
gaggtgcagctgttggagtctgggggaggcttggtacagcctggggggtccctgcgtctctcctgtgtagcctccggattcacctttgatgtggccgagatggattgggtccgccaggctccagggaagggtctagagtgggtctcaactatttcgccgtcgaggagggggacatactacgcagactccgtgaagggccggttcaccatctcccgcgacaattccaagaacacgctgtatctgcaaatgaacagcctgcgtgccgaggacaccgccgtatattactgtgcgaaagcgtacacggggaggagcttgtggggtccgggaaccctggtcaccgtctcgagc

SEQ ID NO: 12 - DNA, 348 bp, Artificial Sequence
  Artificial sequence derived from Homo sapiens sequence
gaggtgcagctgttggagtctgggggaggcttggtacagcctggggggtccctgcgtctctcctgtgcagcctccggattcacctttgatgtggccgagatggattgggtccgccaggctccagggaagggtctagagtgggtctcaactatttcgccgtcgaggagggggacatactacgcagactccgtgaagggccggttcaccatctcccgcgacaattccaagaacacgctgtatctgcaaatgaacagcctgcgtgccgaggacaccgccgtatattactgtgcgaaagcgtatacggggaggagctggtggggtccgggaaccctggtcaccgtctcgagc

SEQ ID NO: 13 - DNA, 348 bp, Artificial Sequence
  Artificial sequence derived from Homo sapiens sequence
gaggtgcacctgttggagtctgggggaggcttggtacagcctggggggtccctgcgtctctcctgcgcagcctccggattcacctttgattccgccgagatggattgggtccgccaggctccagggaagggtctagagtgggtctcaactatttcgccgtcgaggagggggacatactacgcagactccgtgaagggccggttcaccatctcccgcgacaattccaagaacacgctgtatctgcaaatgaacagcctgcgtgccgaggacaccgccatatattactgtgcgaaagcctacaccgggaggagctactggggtccgggaaccctggtcaccgtctcgagc

SEQ ID NO: 14 - DNA, 348 bp, Artificial Sequence
  Artificial sequence derived from Homo sapiens sequence
ggggtgcagctgttggagtctgggggaggcttggtacagcctggggggtccctgcgtctctcctgcgcagcctccggattcacctttgattccgccgagatggattgggtccgccaggctccagggaagggtctagagtgggtctcaactatttcgccgtcgaggagagggacatactacgcagactccgtgaagggccggttcaccatctcccgcgacaattccaagaacacgctgtatctgcaaatgaacagcctgcgtgccgaggacaccgccgtatattactgtgcgaaagcctacaccgggaggagctactggggtccgggaaccctggtcaccgtctcgagc

SEQ ID NO: 15 - DNA, 348 bp, Artificial Sequence
  Artificial sequence derived from Homo sapiens sequence
ggggtgcagctgttggagtctgggggaggcttggtacagcctggggggtccctgcgtctctcctgtgtagcctccggattcacctttgatgtggccgagatggattgggtccgccaggctccagggaagggtctagagtgggtctcaactatttcgccgtcgaggagggggacatactacgcagactccgtgaagggccggttcaccatctcccgcgacaattccaagaacacgctgtatctgcaaatgaacagcctgcgtgccgaggacaccgccgtatattactgtgcgaaagcgtacacggggaggagcttgtggggtccgggaaccctggtcaccgtctcgagc

SEQ ID NO: 16 - DNA, 347 bp, Artificial Sequence
  Consensus
ggkgtrcarctgytggartctggkggwggyytggtwcarcckggkggktcyctgcgtctstcytgygcagcswscggmttcaccttcgcytggtaygatatgggstgggtycgycaggctccrggsaarggtctrgagtgggtstcwwstatygaytggcatggygaggttacmtactaygcrgactcygtkaarggycgkttcaccatctcccgygayaaywscaaraacacsctgtayctgcaratgaacagcctgcgygcygargacaccgcggtataytactgtgcgacmgckgaggaygarccgggktaygactaytggggccagggmacyctggtcacsgt

(19) United States
(12) Patent Application Publication
(10) Pub. No.: US 2012/0093830 A1
(43) Pub. Date: Apr. 19, 2012

(54) SINGLE DOMAIN ANTIBODIES THAT BIND IL-13

(76) Inventor: Inusha De Silva, Cambridgeshire (GB)

(21) Appl. No.: 12/992,718

(22) PCT Filed: May 13, 2009
(86) PCT No.: PCT/EP2009/055745

§ 371 Date: Nov. 15, 2010

Related U.S. Application Data
(63) Continuation of application No. 12/152,903, filed on May 15, 2008, now abandoned.
Application No. 12/152,903 is a continuation-in-part of application No. PCT/GB2007/000228, filed on Jan. 24, 2007.
Application No. PCT/EP2009/055745 is a continuation of application No. 12/397,826, filed on Mar. 4, 2009, now abandoned.
Application No. 12/397,826 is a continuation-in-part of application No. 12/152,903, filed on May 15, 2008, now abandoned.
Application No. 12/152,903 is a continuation-in-part of application No. PCT/GB2007/000228, filed on Jan. 24, 2007.
(60) Provisional application No. 60/761,708, filed on Jan. 24, 2006.
(60) Provisional application No. 60/761,708, filed on Jan. 24, 2006.

(30) Foreign Application Priority Data
Dec. 11, 2008 (EP) PCT/EP2008/067295

(51) Int. Cl. A61K 39/395; C12N 15/63; C12N 5/10; C07K 16/24; C12N 1/19; A61P 35/00; A61P 37/02; C07H 21/04; C12N 1/21

(52) U.S. Cl. 424/158.1; 536/23.53; 435/320.1; 435/325; 435/252.33; 435/252.31; 435/348; 435/254.2; 435/254.21; 435/254.22; 435/254.23; 435/365; 530/389.2

(57) ABSTRACT

Disclosed are ligands that have binding specificity for interleukin-13 (IL-13), or for IL-4 and IL-13. Also disclosed are methods of using these ligands. In particular, the use of these ligands for treating allergic asthma is described.

### BACKGROUND OF THE INVENTION

Reference is made to W02007/085815A2, which is incorporated by reference herein in its entirety.

Interleukin-13 (IL-13) is a pleiotropic cytokine that induces immunoglobulin isotype switching to IgG4 and IgE, CD23 up regulation, VCAM-1 expression, and directly activates eosinphils and mast cells, for example. IL-13 is mainly produced by Th2 cells and inhibits the production of inflammatory cytokines IL-6, TNF, IL-8) by LPS-stimulated monocytes. IL-13 is closely related to IL-4 with which it shares 20-25% sequence similarity at the amino acid level. (Minty et. al., Nature, 363(6417):248-50 (1993)). Although many activities of IL-13 are similar to those of IL-4, IL-13 does not have growth promoting effects on activated T cells or T cells clones as IL-4 does. (Zurawski et al., EMBO J. 12:2663 (1993)).

Interleukin-4 (IL-4) is a pleiotropic cytokine that has a broad spectrum of biological effects on B cells, T cells, and many non-lymphoid cells including monocytes, endothelial cells and fibroblasts. For example, IL-4 stimulates the proliferation of several IL-2- and IL-3-dependent cell lines, induces the expression of class II major histocompatability complex molecules on resting B cells, and enhances the secretion of IgG4 and IgE by human B cells. IL-4 is associated with a Th2-type immune response, and is produced by and promotes differentiation of Th2 cells. IL-4 has been implicated in a number of disorders, such as allergy and asthma.

The cell surface receptors and receptor complexes bind IL-4 and/or IL-13 with different affinities. The principle components of receptors and receptor complexes that bind IL-4 and/or IL-13 are IL-4Rα, IL-13Rα1 and IL-13Rα2. These chains are expressed on the surface of cells as monomers or heterodimers of IL-4Rα/IL-13Rα1 or IL-4Rα/IL-13Rα2. IL-4-rα monomer binds IL-4, but not IL-13. IL-13Rα1 and IL-13Rα2 monomers bind IL-13, but do not bind IL-4. IL-4Rα/IL-13Rα1 and IL-4Rα/IL-13Rα2 heterodimers bind both IL-4 and IL-13.

Th2-type immune responses promote antibody production and humoral immunity, and are elaborated to fight off extracellular pathogens. Th2 cells are mediators of 1 g production (humoral immunity) and produce IL-4, IL-5, IL-6, IL-9, IL-10 and IL-13 (Tanaka, et. al., Cytokine Regulation of Humoral Immunity, 251-272, Snapper, ed., John Wiley and Sons, New York (1996)). Th2-type immune responses are characterized by the generation of certain cytokines (e.g., IL-4, IL-13) and specific types of antibodies (IgE, IgG4) and are typical of allergic reactions, which may result in watery eyes and asthmatic symptoms, such as airway inflammation and contraction of airway muscle cells in the lungs.

Both IL-4 and IL-13 are therapeutically important proteins based on their biological functions. IL-4 has been shown to be able to inhibit autoimmune disease and IL-4 and IL-13 have both shown the potential to enhance anti-tumor immune responses. Since both cytokines are involved in the pathogenesis of allergic diseases, inhibitors of these cytokines could provide therapeutic benefits. Accordingly, a need exists for improved agents that inhibit IL-13, and single agents that inhibit both IL-4 and IL-13.

### SUMMARY OF THE INVENTION

In one aspect the invention provides for the use of DOM10-53-474 (SEQ ID NO:1) to inhibit IL-13 (R130Q variant)-stimulated cell proliferation. DOM10-53-474 is SEQ ID NO:2369 in WO2007/085815A2.

In one embodiment of any aspect of the invention herein, the IL-13 is human IL-13. In one embodiment the IL-13 is human IL-13 with a Q at position 130 (a human IL-13 R130Q variant).

In one aspect the invention provides for the use of DOM10-53-474 (SEQ ID NO:1) to target R130Q IL-13 variant associated with asthma.

In one aspect the invention provides for the use of DOM10-53-474 (SEQ ID NO:1) to target R130Q IL-13 variant associated with bronchial hyperresponsiveness.

In one aspect the invention provides for a polypeptide comprising DOM10-53-474 (SEQ ID NO:1) for inhibiting IL-13 (R130Q variant)-stimulated cell proliferation.

In one aspect the invention provides for a polypeptide comprising DOM10-53-474 (SEQ ID NO:1) for targeting R130Q IL-13 variant associated with asthma.

In one aspect the invention provides for a polypeptide comprising DOM10-53-474 (SEQ ID NO:1) for targeting R130Q IL-13 variant associated with bronchial hyperresponsiveness.

In one aspect the invention provides for the use of DOM10-53-474 (SEQ ID NO:1) in the manufacture of a medicament for inhibiting IL-13 (R130Q variant)-stimulated cell proliferation in a subject. In one embodiment the subject is a human patient.

In one aspect the invention provides for the use of DOM10-53-474 (SEQ ID NO:1) in the manufacture of a medicament for therapy of R130Q IL-13 variant-mediated or—associated asthma in a subject. In one embodiment the subject is a human patient.

In one aspect the invention provides for the use of DOM10-53-474 (SEQ ID NO:1) in the manufacture of a medicament for targeting R130Q IL-13 variant-mediated or—associated asthma in a subject. In one embodiment the subject is a human patient.

In one aspect the invention provides for the use of DOM10-53-474 (SEQ ID NO:1) in the manufacture of a medicament for therapy of R130Q IL-13 variant-mediated or—associated bronchial hyperresponsiveness in a subject. In one embodiment the subject is a human patient.

In one aspect the invention provides for the use of DOM10-53-474 (SEQ ID NO:1) in the manufacture of a medicament for targeting R130Q IL-13 variant-mediated or—associated bronchial hyperresponsiveness in a subject. In one embodiment the subject is a human patient.

In one aspect the invention provides for a method of inhibiting IL-13 (R130Q variant)-stimulated cell proliferation in a patient, eg, a mammal, such as a human, the method comprising administering to the patient a therapeutically effective amount of ligand comprising DOM10-53-474 (SEQ ID NO:1).

In one aspect the invention provides for a method for treating R130Q IL-13 variant-mediated or—associated allergic disease in a patient, the method comprising administering to the patient a therapeutically effective amount of ligand comprising DOM10-53-474 (SEQ ID NO:1).

In one aspect the invention provides for a method for treating R130Q IL-13 variant-mediated or—associated bronchial hyperresponsiveness, the method comprising administering to the patient a therapeutically effective amount of ligand comprising DOM10-53-474 (SEQ ID NO:1).

In one aspect the invention provides for a method for treating a R130Q IL-13 variant-mediated or—associated Th2-type immune response, the method comprising administering to the patient a therapeutically effective amount of ligand comprising DOM10-53-474 (SEQ ID NO:1).

In one aspect the invention provides for a method for treating R130Q IL-13 variant-mediated or—associated asthma, the method comprising administering to the patient a therapeutically effective amount of ligand comprising DOM10-53-474 (SEQ ID NO:1).

In one aspect the invention provides for a nucleic acid comprising a codon-optimised sequence that encodes DOM10-53-474 (SEQ ID NO:1), optionally wherein the sequence is codon-optimised for expression in E coli or Pichia pastoris. In one embodiment, the codon-optimised sequence is selected from SEQ ID NO: 3, SEQ ID NO: 4 and SEQ ID NO: 5.

In one aspect the invention provides for a freeze-dried or lyophilized formulation comprising DOM10-53-474 (SEQ ID NO:1).

In one aspect the invention provides for a formulation of DOM10-53-474 (SEQ ID NO:1) for nebulisation, wherein the formulation shows no aggregation peaks as determined using a 2000 Mwt cut-off SEC (size exclusion chromatography) column (eg, a TSKgeL G2000SWXL SEC column) with SEC performed at 0.5 mL/min for 45 minutes with PBS (phosphate buffered saline)+10% EtOH as the mobile phase.

In one aspect the invention provides for a formulation of DOM10-53-474 (SEQ ID NO:1) for nebulisation, wherein the formulation comprises PEG.

In one aspect the invention provides for a formulation of DOM10-53-474 (SEQ ID NO:1) for nebulisation, wherein the formulation comprises particles with a mean median aerodynamic diameter (MMAD) of about 5.20 μm or less. Optionally the MMAD is from about 5.20 μm to about 3.66 μm, optionally from about 5.20 μm to about 4.10 μm, optionally from about 4.43 μm or less, optionally from about 4.43 μm to about 3.66 μm.

In one aspect the invention provides for a formulation of DOM10-53-474 (SEQ ID NO:1) for nebulisation, wherein the formulation comprises particles with 47.9% or more of the particles being in the size range of < about 5 μm. Optionally about 56.6% or more, about 60.6% or more, about 61.2% or more, about 63.8% or more, or about 66.5% or more of the particles are in the size range of < about 5 μm.

The invention relates to ligands that have binding specificity for IL-13 (e.g., human IL-13), and to ligands that have binding specificity for IL-4 and IL-13 (e.g., human IL-4 and human IL-13). For example, the ligand can comprise a polypeptide domain having a binding site with binding specificity for IL-13, or comprise a polypeptide domain having a binding site with binding specificity for IL-4 and a polypeptide domain having a binding site with binding specificity for IL-13.

In one aspect, the invention relates to a ligand that has binding specificity for IL-4 and for IL-13. Such ligands comprise a protein moiety that has a binding site with binding specificity for IL-4 and a protein moiety that has a binding site with binding specificity for IL-13. The protein moiety that has a binding site with binding specificity for IL-4 and the protein moiety that has a binding site with binding specificity for IL-13 can be any suitable binding moiety. The protein moieties can be a peptide moiety, polypeptide moiety or protein moiety. For example, the protein moieties can be provided by an antibody fragment that has a binding site with binding specificity for IL-4 or IL-13, such as an immunoglobulin single variable domain that has binding specificity for IL-4 or IL-13.

The ligand can comprise a protein moiety that has a binding site with binding specificity for IL-13 (e.g., an immunoglobulin single variable domain) that competes for binding to IL-13 with an anti-IL-13 domain antibody (dAb) selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10), and optionally DOM10-53-474 (SEQ ID NO:1). For example, the binding of the protein moiety that has a binding site with binding specificity for IL-13 to IL-13 can be inhibited by a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10), and optionally DOM10-53-474 (SEQ ID NO:1). The protein moiety that has a binding site with binding specificity for IL-13 can have the epitopic specificity of a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10), and optionally DOM10-53-474 (SEQ ID NO:1).

The invention provides ligand can comprise a protein moiety that has a binding site with binding specificity for IL-13 (e.g., an immunoglobulin single variable domain) that competes for binding to IL-13 with an anti-IL-13 domain antibody (dAb) selected from the group consisting of DOM10-275-78 (SEQ ID NO:6) (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10). For example, the binding of the protein moiety that has a binding site with binding specificity for IL-13 to IL-13 can be inhibited by a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10). The protein moiety that has a binding site with binding specificity for IL-13 can have the epitopic specificity of a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10).

The ligand that has binding specificity for IL-4 and IL-13 can inhibit binding of IL-4 to IL-4R, inhibit the activity of IL-4, and/or inhibit the activity of IL-4 without substantially inhibiting binding of IL-4 to IL-4R.

In one embodiment, the ligand (e.g., immunoglobulin single variable domain) that binds IL-4 inhibits binding of IL-4 to an IL-4 receptor (e.g., IL-4Rα) with an inhibitory concentration 50 (IC50) that is ≦10 μM, ≦1 μM, ≦100 nM, ≦10 nM, ≦1 nM, ≦500 μM, ≦300 μM, ≦100 μM, or ≦10 μM. The IC50 is in one embodiment determined using an in vitro receptor binding assay, such as the assay described herein.

It is also possible that the ligand (e.g., immunoglobulin single variable domain) that binds an IL-4 receptor inhibits IL-4 induced functions in a suitable in vitro assay with a neutralizing dose 50 (ND50) that is ≦10 μM, ≦1≦100 nM, ≦10 nM, ≦1 nM, ≦500 μM, ≦300 μM, ≦100 μM, or ≦10 μM. For example, the ligand that binds an IL-4 receptor can inhibit IL-4 induced proliferation of TF-1 cells (ATCC Accession No. CRL-2003) in an in vitro assay, such as the assay described herein.

It is also possible that the ligand (e.g., immunoglobulin single variable domain) that binds an IL-4 receptor inhibits house dust mite (HDM) induced proliferation of peripheral blood mononuclear cells (PBMC) by at least about 20%, at least about 30%, at least about 40%, at least about 50%, at least about 60%, at least about 70%, at least about 80%, or at least about 90% in a suitable in vitro assay, such as the assay described herein where 4×10⁶ cells/ml are stimulated with 20-50 ug/ml HDM and 100 nM anti-IL-4 dAbs are added.

The invention provides a ligand (e.g., immunoglobulin single variable domain) that does not substantially inhibit binding of IL-4 to an IL-4 receptor (e.g., IL-4Rα) does not significantly inhibit binding of IL-4 to an IL-4 receptor in the receptor binding assay is described herein. For example, such a ligand might inhibit binding of IL-4 to an IL-4 receptor in the receptor binding assay described herein with an IC50 of about 1 mM or higher or inhibits binding by no more than about 20%, no more than about 15%, no more than about 10%, or no more than about 5%.

The ligand that has binding specificity for IL-4 and IL-13 can inhibit binding of IL-13 to IL-13Rα1 and/or IL-13Rα2, inhibit the activity of IL-13, and/or inhibit the activity of IL-13 without substantially inhibiting binding of IL-13 to IL-13Rα1 and/or IL-13Rα2.

In one embodiment, the ligand (e.g., immunoglobulin single variable domain) that binds IL-13 inhibits binding of IL-13 to an IL-13 receptor (e.g., IL-13Rα1, IL-13Rα2) with an inhibitory concentration 50 (IC50) that is ≦10 μM, ≦1 μM, ≦100 nM, ≦10 nM, ≦1 nM, ≦500 μM, ≦300 μM, ≦100 μM, or ≦10 μM. The IC50 is in one embodiment determined using an in vitro receptor binding assay, such as the assay described herein.

It is also possible that the ligand (e.g., immunoglobulin single variable domain) that binds an IL-13 receptor inhibits IL-13 induced functions in a suitable in vitro assay with a neutralizing dose 50 (ND50) that is ≦10 μM, ≦1 μM, ≦100 nM, ≦10 nM, ≦1 nM, ≦500 pM, ≦300 pM, ≦100 pM, ≦10 pM, ≦1 pM≦500 fM, ≦300 fM, ≦100 fM, ≦10 fM. For example, the ligand that binds an IL-13 receptor can inhibit IL-13 induced proliferation of TF-1 cells (ATCC Accession No. CRL-2003) in an in vitro assay, such as the assay described herein wherein TF-1 cells were mixed with 5 ng/ml final concentration of IL-13.

It is also possible that the ligand that binds an IL-13 receptor inhibits IL-13 induced B cell proliferation by at least about 50%, at least about 60%, at least about 70%, at least about 80%, or at least about 90% in an in vitro assay, such as the assay described herein where 1×10⁵ B cells were incubated with 10 or 100 nM anti-IL-13 dAbs.

The invention provides a ligand (e.g., immunoglobulin single variable domain) that does not substantially inhibit binding of IL-13 to an IL-13 receptor (e.g., IL-13Rα1, IL-13Rα2) does not significantly inhibit binding of IL-13 to an IL-13 receptor in the receptor binding assay or sandwich ELISA described herein. For example, such a ligand might inhibit binding of IL-13 to an IL-13 receptor in the receptor binding assay described herein with an IC50 of about 1 mM or higher or inhibit binding by no more than about 20%, no more than about 15%, no more than about 10%, or no more than about 5%.

In more particular embodiments, the ligand that has binding specificity for IL-4 and for IL-13 comprises an immunoglobulin single variable domain with binding specificity for IL-4 and an immunoglobulin single variable domain with binding specificity for IL-13, wherein an immunoglobulin single variable domain with binding specificity for IL-4 competes for binding to IL-4 with an anti-IL-4 domain antibody (dAb) selected from the group of anti-IL-4 dAbs disclosed herein.

In more particular embodiments, the ligand that has binding specificity for IL-4 and for IL-13 comprises an immunoglobulin single variable domain with binding specificity for IL-4 and an immunoglobulin single variable domain with binding specificity for IL-13, wherein an immunoglobulin single variable domain with binding specificity for IL-13 competes for binding to IL-13 with an anti-IL-13 domain antibody (dAb) selected from the group consisting of the anti-IL-13 dAbs disclosed herein.

The ligand that has binding specificity for IL-4 and IL-13 can contain a protein binding moiety (e.g., immunoglobulin single variable domain) with binding specificity for IL-4 that binds IL-4 with an affinity (KD) that is between about 100 nM and about 1 pM, as determined by surface plasmon resonance.

The ligand that has binding specificity for IL-4 and IL-13 can contain a protein binding moiety (e.g., immunoglobulin single variable domain) with binding specificity for IL-13 that binds IL-13 with an affinity (KD) that is between about 100 nM and about 1 pM, as determined by surface plasmon resonance.

The ligand that has binding specificity for IL-4 and IL-13 can bind IL-4 with an affinity (KD) that is between about 100 nM and about 1 pM, as determined by surface plasmon resonance.

The ligand that has binding specificity for IL-4 and IL-13 can bind IL-13 with an affinity (KD) that is between about 100 nM and about 1 pM, as determined by surface plasmon resonance.

The ligand that has binding specificity for IL-4 and IL-13 can comprise an immunoglobulin single variable domain with binding specificity for IL-4 and an immunoglobulin single variable domain with binding specificity for IL-13, wherein the immunoglobulin single variable domains are selected from the group consisting of a human V_{H} and a human V_{L}.

In some embodiments, the ligand that has binding specificity for IL-4 and IL-13 can be an IgG-like format comprising two immunoglobulin single variable domains with binding specificity for IL-4, and two immunoglobulin single variable domains with binding specificity for IL-13.

In some embodiments, the ligand that has binding specificity for IL-4 and for IL-13 can comprise an antibody Fc region.

In some embodiments, the ligand that has binding specificity for IL-4 and IL-13 can comprise an IgG constant region.

The invention also relates to a ligand that has binding specificity for IL-13 comprising an immunoglobulin single variable domain with binding specificity for IL-13, wherein the immunoglobulin single variable domain with binding specificity for IL-13 competes for binding to IL-13 with an anti-IL-13 domain antibody (dAb) selected from the group consisting of the anti-IL-13 dAbs disclosed herein. For example, the immunoglobulin single variable domain with binding specificity for IL-13 can comprise an amino acid sequence that has at least about 70%, at least about 75%, at least about 80% or at least about 85% amino acid sequence identity with the amino acid sequence of a dAb selected from the group consisting of the anti-IL-13 dAbs disclosed herein. In other examples, the binding of the immunoglobulin single variable domain with binding specificity for IL-13 to IL-13 is inhibited by a dAb selected from the group consisting of DOM10-275-78 (SEQ ID NO:6) (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10), and optionally DOM10-53-474 (SEQ ID NO:1). In other examples, the immunoglobulin single variable domain with binding specificity for IL-13 has the epitopic specificity of a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10), and optionally DOM10-53-474 (SEQ ID NO:1).

The ligand that has binding specificity for IL-13 can inhibit binding of IL-13 to IL-13Rα1 and/or IL-13Rα2, inhibit the activity of IL-13, and/or inhibit the activity of IL-13 without substantially inhibiting binding of IL-13Rα1 and/or IL-13Rα2 to IL-13.

In one embodiment, the ligand (e.g., immunoglobulin single variable domain) that binds IL-13 inhibits binding of IL-13 to an IL-13 receptor (e.g., IL-13Rα1, IL-13R2) with an inhibitory concentration 50 (IC50) that is ≦10 μM, ≦1 μM, ≦100 nM, ≦10 nM, ≦1 nM, ≦500 pM, ≦300 pM, ≦100 pM, or ≦10 pM. The IC50 is in one embodiment determined using an in vitro receptor binding assay, such as the assay described herein.

It is also possible that the ligand (e.g., immunoglobulin single variable domain) that binds an IL-13 receptor inhibits IL-13 induced functions in a suitable in vitro assay with a neutralizing dose 50 (ND50) that is ≦10 μM, ≦1 μM, ≦100 nM, ≦10 nM, ≦1 nM, ≦500 pM, ≦300 pM, ≦100 pM, ≦10 pM, ≦1 pM≦500 fM, ≦300 fM, ≦100 fM, ≦10 fM. For example, the ligand that binds an IL-13 receptor can inhibit IL-13 induced proliferation of TF-1 cells (ATCC Accession No. CRL-2003) in an in vitro assay, such as the assay described herein wherein TF-1 cells were mixed with 5 ng/ml final concentration of IL-13.

It is also possible that the ligand that binds an IL-13 receptor inhibits IL-13 induced B cell proliferation by at least at least about 70%, at least about 80%, or at least about 90% in an in vitro assay, such as the assay described herein where 1×10⁵ B cells were incubated with 10 or 100 nM anti-IL-13 dAbs.

The invention provides a ligand (e.g., immunoglobulin single variable domain) that does not substantially inhibit binding of IL-13 to an IL-13 receptor (e.g., IL-13Rα1, IL-13Rα2) does not significantly inhibit binding of IL-13 to an IL-13 receptor in the receptor binding assay or sandwich ELISA described herein. For example, such a ligand might inhibit binding of IL-13 to an IL-13 receptor in the receptor binding assay described herein with an IC50 of about 1 mM or higher or inhibit binding by no more than about 20%, no more than about 15%, no more than about 10%, or no more than about 5%.

The ligand that has binding specificity for IL-13 can contain an immunoglobulin single variable domain with binding specificity for IL-13 that binds IL-13 with an affinity (KD) that is between about 100 nM and about 1 pM, as determined by surface plasmon resonance.

The ligand that has binding specificity for IL-13 can bind IL-13 with an affinity (KD) that is between about 100 nM and about 1 pM, as determined by surface plasmon resonance.

The ligand that has binding specificity for IL-13 can comprise an immunoglobulin single variable domain with binding specificity for IL-13 that is selected from the group consisting of a human V_{H} and a human V_{L}.

In some embodiments, the ligand that has binding specificity for IL-13 is an IgG-like format comprising at least two immunoglobulin single variable domains with binding specificity for IL-13.

In some embodiments, the ligand that has binding specificity for IL-13 comprises an antibody Fc region.

In some embodiments, the ligand that has binding specificity for IL-13 comprises an IgG constant region.

The invention also relates to a ligand (e.g., a fusion protein) that has binding specificity for IL-4 and IL-13, comprising an immunoglobulin single variable domain with binding specificity for IL-4, wherein an immunoglobulin single variable domain with binding specificity for IL-4 competes for binding to IL-4 with an anti-IL-4 domain antibody (dAb) selected from the group consisting of the anti-IL-4 dAbs disclosed herein and comprising an immunoglobulin single variable domain with binding specificity for IL-13, wherein an immunoglobulin single variable domain with binding specificity for IL-13 competes for binding to IL-13 with an anti-IL-13 domain antibody (dAb) selected from the group consisting of the anti-IL-13 dAbs disclosed herein.

For example, the ligand (e.g., fusion protein) comprising an immunoglobulin single variable domain with binding specificity for IL-4 can comprise an amino acid sequence that has at least 85% amino acid sequence identity with the amino acid sequence of a dAb selected from the group consisting of the anti-IL-4 dAbs disclosed herein.

In another example, the ligand (e.g., fusion protein) comprising an immunoglobulin single variable domain with binding specificity for IL-13 can comprise an amino acid sequence that has at least 85% amino acid sequence identity with the amino acid sequence of a dAb selected from the group consisting of the anti-IL-13 dAbs disclosed herein.

In some embodiments, the ligand (e.g., fusion protein) comprising an immunoglobulin single variable domain with binding specificity for IL-4 and an immunoglobulin single variable domain with binding specificity for IL-3 further comprises a linker moiety.

In some embodiments, the ligand comprises a protein moiety that has a binding site that binds IL-13, wherein said protein moiety comprises an amino acid sequence that is the same as the amino acid sequence of CDR3 of an anti-IL-13 dAb disclosed herein.

In other embodiments, the ligand comprises a protein moiety that has a binding site that binds IL-13, wherein said protein moiety comprises an amino acid sequence that is the same as the amino acid sequence of CDR3 of an anti-IL-13 dAb disclosed herein and has an amino acid sequence that is the same as the amino acid sequence of CDR1 and/or CDR2 of an anti-IL-13 dAb disclosed herein.

In other embodiments, the ligand comprises an immunoglobulin single variable domain that binds IL-13, wherein the amino acid sequence of the immunoglobulin single variable domain that binds IL-13 differs from the amino acid sequence of an anti-IL-13 dAb disclosed herein at no more than 25 amino acid positions and has a CDR1 sequence that has at least 50% identity to the CDR1 sequences of the anti-IL-13 dAbs disclosed herein.

In other embodiments, the ligand comprises an immunoglobulin single variable domain that binds IL-13, wherein the amino acid sequence of the immunoglobulin single variable domain that binds IL-13 differs from the amino acid sequence of an anti-IL-13 dAb disclosed herein at no more than 25 amino acid positions and has a CDR2 sequence that has at least 50% identity to the CDR2 sequences of the anti-IL-13 dAbs disclosed herein.

In other embodiments, the ligand comprises an immunoglobulin single variable domain that binds IL-13, wherein the amino acid sequence of the immunoglobulin single variable domain that binds IL-13 differs from the amino acid sequence of an anti-IL-13 dAb disclosed herein at no more than 25 amino acid positions and has a CDR3 sequence that has at least 50% identity to the CDR3 sequences of the anti-IL-13 dAbs disclosed herein.

In other embodiments, the ligand comprises an immunoglobulin single variable domain that binds IL-13, wherein the amino acid sequence of the immunoglobulin single variable domain that binds IL-13 differs from the amino acid sequence of an anti-IL-13 dAb disclosed herein at no more than 25 amino acid positions and has a CDR1 sequence and a CDR2 sequence that has at least 50% identity to the CDR1 and CDR2 sequences, respectively, of the anti-IL-13 dAbs disclosed herein.

In other embodiments, the ligand comprises an immunoglobulin single variable domain that binds IL-13, wherein the amino acid sequence of the immunoglobulin single variable domain that binds IL-13 differs from the amino acid sequence of an anti-IL-13 dAb disclosed herein at no more than 25 amino acid positions and has a CDR2 sequence and a CDR3 sequence that has at least 50% identity to the CDR2 and CDR3 sequences, respectively, of the anti-IL-13 dAbs disclosed herein.

In other embodiments, the ligand comprises an immunoglobulin single variable domain that binds IL-13, wherein the amino acid sequence of the immunoglobulin single variable domain that binds IL-13 differs from the amino acid sequence of an anti-IL-13 dAb disclosed herein at no more than 25 amino acid positions and has a CDR1 sequence and a CDR3 sequence that has at least 50% identity to the CDR1 and CDR3 sequences, respectively, of the anti-IL-13 dAbs disclosed herein.

In other embodiments, the ligand comprises an immunoglobulin single variable domain that binds IL-13, wherein the amino acid sequence of the immunoglobulin single variable domain that binds IL-13 differs from the amino acid sequence of an anti-IL-13 dAb disclosed herein at no more than 25 amino acid positions and has a CDR1 sequence, CDR2 sequence and a CDR3 sequence that has at least 50% identity to the CDR1, CDR2 and CDR3 sequences, respectively, of the anti-IL-13 dAbs disclosed herein.

In another embodiment, the invention is a ligand comprising an immunoglobulin single variable domain that binds IL-13, wherein the immunoglobulin single variable domain comprises a CDR2 sequence that has at least 50% identity to the CDR2 sequence of an anti-IL-13 dAb disclosed herein.

In another embodiment, the invention is a ligand comprising an immunoglobulin single variable domain that binds IL-13, wherein the immunoglobulin single variable domain comprises a CDR3 sequence that has at least 50% identity to the CDR3 sequence of an anti-IL-13 dAb disclosed herein.

In another embodiment, the invention is a ligand comprising an immunoglobulin single variable domain that binds IL-13, wherein the immunoglobulin single variable domain comprises a CDR1 and a CDR2 sequence that has at least 50% identity to the CDR1 and CDR2 sequences, respectively, of an anti-IL-13 dAb disclosed herein.

In another embodiment, the invention is a ligand comprising an immunoglobulin single variable domain that binds IL-13, wherein the immunoglobulin single variable domain comprises a CDR2 and a CDR3 sequence that has at least 50% identity to the CDR2 and CDR3 sequences, respectively, of an anti-IL-13 dAb disclosed herein.

In another embodiment, the invention is a ligand comprising an immunoglobulin single variable domain that binds IL-13, wherein the immunoglobulin single variable domain comprises a CDR1 and a CDR3 sequence that has at least 50% identity to the CDR1 and CDR3 sequences, respectively, of an anti-IL-13 dAb disclosed herein.

In another embodiment, the invention is a ligand comprising an immunoglobulin single variable domain that binds IL-13, wherein the immunoglobulin single variable domain comprises a CDR1, CDR2, and a CDR3 sequence that has at least 50% identity to the CDR1, CDR2, and CDR3 sequences, respectively, of an anti-IL-13 dAb disclosed herein.

In other embodiments, any of the ligands described herein further comprises a half-life extending moiety, such as a polyalkylene glycol moiety, serum albumin or a fragment thereof, transferrin receptor or a transferrin-binding portion thereof, or a moiety comprising a binding site for a polypeptide that enhance half-life in vivo. In some embodiments, the half-life extending moiety is a moiety comprising a binding site for a polypeptide that enhances half-life in vivo selected from the group consisting of an affibody, a SpA domain, an LDL receptor class A domain, an EGF domain, and an avimer. In an embodiment, the half-life extending moiety is a moiety comprising a non-Ig scaffold and a binding site for a polypeptide (eg, serum albumin, such as human serum albumin) that enhances half-life in vivo, optionally wherein the scaffold is selected from one of the following scaffolds.

### Non-Ig Scaffolds

CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies. For further details see Journal of Immunological Methods 248 (1-2), 31-45 (2001)

Lipocalins are a family of extracellular proteins which transport small hydrophobic molecules such as steroids, bilins, retinoids and lipids. They have a rigid β-sheet secondary structure with a numer of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633

An affibody is a scaffold derived from Protein A of Staphylococcus aureus which can be engineered to bind to antigen. The domain consists of a three-helical bundle of approximately 58 amino acids. Libraries have been generated by randomisation of surface residues. For further details see Protein Eng. Des. Sel. 17, 455-462 (2004) and EP1641818A1

Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007)

A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem. 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomising residues in the first α-helix and a β-turn of each repeat. Their binding interface can be increased by increasing the number of modules (a method of affinity maturation). For further details see J. Mol. Biol. 332, 489-503 (2003), PNAS100(4), 1700-1705 (2003) and J. Mol. Biol. 369, 1015-1028 (2007) and US20040132028A1.

Fibronectin is a scaffold which can be engineered to bind to antigen. Adnectins consists of a backbone of the natural amino acid sequence of the 10th domain of the repeating units of human fibronectin type III (FN3). Three loops at one end of the β-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther.

5, 783-797 (2005).

Microbodies are derived from naturally occurring microproteins of 25-50 amino acids in length which contain 3-4 cysteine bridges—examples of microproteins include KalataB1 and conotoxin and knottins. The microproteins have a loop which can be engineered to include upto 25 amino acids without affecting the overall fold of the microprotein. For further details of engineered knottin domains, see WO2008098796.

Other epitope binding domains include proteins which have been used as a scaffold to engineer different target antigen binding properties include human γ-crystallin and human ubiquitin (affilins), kunitz type domains of human protease inhibitors, PDZ-domains of the Ras-binding protein AF-6, scorpion toxins (charybdotoxin), C-type lectin domain (tetranectins) are reviewed in Chapter 7—Non-Antibody Scaffolds from Handbook of Therapeutic Antibodies (2007, edited by Stefan Dubel) and Protein Science 15:14-27 (2006). Epitope binding domains of the present invention could be derived from any of these alternative protein domains.

In other embodiments, the half-life extending moiety is a polyethylene glycol moiety.

In other embodiments, the half-life extending moiety is an antibody or antibody fragment (e.g., an immunoglobulin single variable domain) comprising a binding site for serum albumin or neonatal Fc receptor.

The invention also relates to a ligand of the invention for use in therapy or diagnosis, and to the use of a ligand of the invention for the manufacture of a medicament for treatment, prevention or suppression of a disease described herein (e.g., allergic disease, Th2-mediated disease, asthma, cancer).

The invention also relates to a ligand of the invention for use in treating, suppressing or preventing a Th2-type immune response.

The invention also relates to therapeutic methods that comprise administering a therapeutically effective amount of a ligand of the invention to a subject in need thereof. In one embodiment, the invention relates to a method for inhibiting a Th2-type immune response comprising administering to a subject in need thereof a therapeutically effective amount of a ligand of the invention.

In other embodiments, the invention relates to a method for treating asthma comprising administering to a subject in need thereof a therapeutically effective amount of a ligand of the invention.

In other embodiments, the invention relates to a method for treating cancer comprising administering to a subject in need thereof a therapeutically effective amount of a ligand of the invention.

The invention also relates to the use of any of the ligands of the invention for the manufacture of a medicament for simultaneous administration of an anti-IL-4 treatment and an anti-IL-13 treatment. In other embodiments, the invention relates to a method of administering to a subject anti-IL-4 treatment and anti-IL-13 treatment, comprising simultaneous administration of an anti-IL-4 treatment and an anti-IL-13 treatment by administering to the subject a therapeutically effective amount of a ligand that has binding specificity for IL-4 and IL-13.

The invention also relates to a composition (e.g., pharmaceutical composition) comprising a ligand of the invention and a physiologically acceptable carrier. In some embodiments, the composition comprises a vehicle for intravenous, intramuscular, intraperitoneal, intraarterial, intrathecal, intraarticular, subcutaneous administration, pulmonary, intranasal, vaginal, or rectal administration.

The invention also relates to a drug delivery device comprising the composition (e.g., pharmaceutical composition) of the invention. In some embodiments, the drug delivery device comprises a plurality of therapeutically effective doses of ligand.

In other embodiments, the drug delivery device is selected from the group consisting of parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, rectal delivery device, syringe, a transdermal delivery device, a capsule, a tablet, a nebulizer, an inhaler, an atomizer, an aerosolizer, a mister, a dry powder inhaler, a metered dose inhaler, a metered dose sprayer, a metered dose mister, a metered dose atomizer, and a catheter.

The invention also relates to an isolated or recombinant nucleic acid encoding any of the ligands of the invention. In other embodiments, the invention relates to a vector comprising the recombinant nucleic acid of the invention.

The invention also relates to a host cell comprising the recombinant nucleic acid of the invention or the vector of the invention.

The invention also relates to a method for producing a ligand, comprising maintaining a host cell of the invention under conditions suitable for expression of a nucleic acid or vector of the invention, whereby a ligand is produced. In other embodiments, the method of producing a ligand further comprises isolating the ligand.

The invention also relates to a method of inhibiting proliferation of peripheral blood mononuclear cells (PBMC) in an allergen-sensitized subject, comprising administering to a subject a pharmaceutical composition comprising any of the ligands of the invention. In some embodiments, the allergen is selected from house dust mite, cat allergen, grass allergen, mold allergen, and pollen allergen.

The invention also relates to a method of inhibiting proliferation of B cells in a subject, comprising administering to the subject a pharmaceutical composition comprising a ligand of the invention.

The invention also relates to a pharmaceutical composition for treating preventing or suppressing a disease as described herein (e.g., Th2-mediated disease, allergic disease, asthma, cancer), comprising as an active ingredient a ligand as described herein.

The invention also relates to a ligand that has binding specificity for IL-4 and IL-13 comprising a protein moiety that has a binding site with binding specificity for IL-4, and a protein moiety that has a binding site with binding specificity for IL-13, wherein the protein moiety that has binding specificity for IL-4 does not compete for binding with any of the anti-IL-4 dAbs disclosed herein.

The invention also relates to a ligand that has binding specificity for IL-4 and IL-13 comprising a protein moiety that has a binding site with binding specificity for IL-4, and a protein moiety that has a binding site with binding specificity for IL-13, wherein the protein moiety that has binding specificity for IL-13 does not compete for binding with any of the anti-IL-13 dAbs disclosed herein.

The invention also relates to a ligand that has binding specificity for IL-4 and IL-13, wherein the ligand is a fusion protein comprising an immunoglobulin single variable domain with binding specificity for IL-4 and an immunoglobulin single variable domain with binding specificity for IL-13, wherein the immunoglobulin single variable domain with binding specificity for IL-4 competes for binding to IL-4 with an anti-IL-4 domain antibody (dAb) selected from the group consisting of DOM9-1,2-210 and DOM9-155-78, and the immunoglobulin single variable domain with binding specificity for IL-13 competes for binding to IL-13 with an anti-IL-13 domain antibody (dAb) selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10), and optionally DOM10-53-474 (SEQ ID NO:1).

The invention also relates to a ligand that has binding specificity for IL-4 and IL-13, wherein the ligand is a fusion protein comprising an immunoglobulin single variable domain with binding specificity for IL-4 and an immunoglobulin single variable domain with binding specificity for IL-13, wherein the immunoglobulin single variable domain with binding specificity for IL-4 competes for binding to IL-4 with an anti-IL-4 domain antibody (dAb) selected from the group consisting of DOM9-1,2-210 and DOM9-155-78, and the immunoglobulin single variable domain with binding specificity for IL-13 competes for binding to IL-13 with an anti-IL-13 domain antibody (dAb) selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10).

In some embodiments, the invention relates to a ligand that has binding specificity for IL-13, comprising an immunoglobulin single variable domain with binding specificity for human IL-13 and a non-human IL-13. In possible embodiments, the non-human IL-13 is selected from rhesus IL-13 and cynomolgous IL-13. It is also possible that the binding affinity of the immunoglobulin single variable domain for non-human IL-13 and the binding affinity for human IL-13 differ by no more than a factor of 10, 50, 100, 500 or 1000.

In other embodiments, the invention relates to a ligand that has binding specificity for IL-4 and IL-13, comprising an immunoglobulin single variable domain with binding specificity for IL-4 and an immunoglobulin single variable domain with binding specificity for IL-13, wherein the immunoglobulin single variable domain with binding specifity for IL-4 binds human IL-4 and a non-human IL-4 and the immunoglobulin single variable domain with binding specificity for IL-13 binds human IL-13 and a non-human IL-13. In possible embodiments, the non-human IL-4 is selected from rhesus IL-4 and cynomolgous IL-4 and the non-human IL-13 is selected from rhesus IL-13 and cynomolgous IL-13. It is also possible that the binding affinity of the immunoglobulin single variable domain for non-human IL-4 and the binding affinity for human IL-4 differ by no more than a factor of 10, 50, 100, 500 or 1000, and the binding affinity of the immunoglobulin single variable domain for non-human IL-13 and the binding affinity for human IL-13 differ by no more than a factor of 10, 50, 100, 500 or 1000.

The amino acid and nucleotide sequences of DOM10-53-474 and variants thereof, DOM10-275-78, DOM10-275-94, DOM10-275-99, DOM10-275-100 and DOM10-275-101 are set out below. All other single variable domain sequences quoted by SEQ ID NO are disclosed in WO2007/085815A2, which are incorporated herein in their entirety by reference as though explicitly reproduced herein, including to provide disclosure for incorporation into claims herein.

The amino acid sequence of DOM10-53-474 (SEQ ID NO:1) is disclosed as SEQ ID NO: 2369 in WO2007/085815A2 and is as follows:—

Gly Val Gln Leu Leu Glu Ser Gly Gly Gly Leu Val Gln Pro Gly Gly
Ser Leu Arg Leu Ser Cys Ala Ala Ser Gly Phe Thr Phe Ala Trp Tyr
Asp Met Gly Trp Val Arg Gln Ala Pro Gly Lys Gly Leu Glu Trp Val
Ser Ser Ile Asp Trp His Gly Glu Val Thr Tyr Tyr Ala Asp Ser Val
Lys Gly Arg Phe Thr Ile Ser Arg Asp Asn Ser Lys Asn Thr Leu Tyr
Leu Gln Met Asn Ser Leu Arg Ala Glu Asp Thr Ala Val Tyr Tyr Cys
Ala Thr Ala Glu Asp Glu Pro Gly Tyr Asp Tyr Trp Gly Gln Gly Thr
Leu Val Thr Val Ser Ser

The nucleotide sequence of DOM10-53-474 (SEQ ID NO:2) is disclosed as SEQ ID NO: 2105 in WO2007/085815A2 and is as follows:—

ggggtgcagc tgttggagtc tgggggaggc ttggtacagc ctggggggtc cctgcgtctc tcctgtgcag
cctccggatt caccttcgct tggtatgata tggggtgggt ccgccaggct ccagggaagg gtctagagtg
ggtctcaagt attgattggc atggtgaggt tacatactac gcagactccg tgaagggccg gttcaccatc
tcccgcgaca attccaagaa cacgctgtat ctgcaaatga acagcctgcg tgccgaggac accgcggtat
attactgtgc gacagcggag gacgagccgg ggtatgacta ctggggccag ggaaccctgg tcaccgtctc tagc

### BRIEF DESCRIPTION OF THE DRAWINGS

The figures and section entitled “BRIEF DESCRIPTION OF THE DRAWINGS” as they appear in WO2007/085815A2 are incorporated herein in their entirety by reference as though explicitly reproduced herein. All of the amino acid and nucleotide sequences disclosed in WO2007/085815A2 are incorporated herein by reference as though written verbatim herein, and to provide explicit support to recite one or more of these sequences in the claims herein.

FIG. 1A is a graph showing the percent inhibition of human IL-13 (3 ng/ml) stimulated alkaline phosphatase production in HEK STAT6 cells (HEK293 cells stably transfected with the STAT6 gene). The potencies for the anti-IL-13 dAbs DOM10-53-474 and DOM10-275-78 were 0.63 nM and 2.5 nM respectively. X axis=[dab] nM; Y-axis=% inhibition.

EC50: DOM10-275-78(JAL050308_{—}20s)=2.422; DOM10-275-78(JAL050308_{—}20p)=2.645; DOM10-53-474=0.7198.

EC50: DOM10-275-78(JAL050308_{—}20s)=2.160 to 2.715; DOM10-275-78(JAL050308_{—}20p)=2.436 to 2.871; DOM10-53-474=0.6579 to 0.7875.

FIG. 1B is a graph showing the percent inhibition of cynomolgus IL-13 (3 ng/ml) stimulated alkaline phosphatase production in HEK STAT6 cells (HEK293 cells stably transfected with the STAT6 gene). The potencies for the anti-IL-13 dAbs DOM10-53-474 and DOM10-275-78 were 11.1 nM and 1.4 nM respectively. X axis=[dab] nM; Y-axis=% inhibition.

EC50: DOM10-275-78(JAL050308_{—}20s)=1.372; DOM10-275-78(JAL050308_{—}20p)=1.369; DOM10-53-474=10.81.
EC50: DOM10-275-78(JAL050308_{—}200=1.372; DOM10-275-78(JAL050308_{—}20p)=1.3691; DOM10-53-474=8.618 to 13.56.
FIG. 2 is a size exclusion chromatography (SEC)-MALLS trace of DOM10-275-78 showing a single peak. The molar mass is the same across the whole width, approximately 13 kDa, meaning that the DOM10-275-78 molecule is mostly a monomer. About 90% of the injected protein was eluted off the column. X axis=time (minutes); Y-axis=molar mass (g/mol). The last number on the X axis is “16.0”.

FIG. 3 is a SEC-MALLS trace of DOM10-53-474 showing a single peak, with the molar mass defined as 13 kDa in the right part of the peak, but increasing over the left part of the peak to 18 kDa. This indicates that the majority of the protein is monomer. X axis time (minutes); Y-axis=molar mass (g/mol).

| | Peak 2 |
|---|---|
| Polydispersity | |
| Mw/Mn | 1.001 (13%) |
| Mz/Mn | 1.003 (23%) |
| Molar mass moments (g/mol) | |
| Mn | 1.399e+4 (9%) |
| Mw | 1.401e+4 (9%) |
| Mz | 1.403e+4 (22%) |

FIG. 4 is a differential scanning calorimetry (DSC) trace of DOM10-275-78 in PBS. The fitted data shows a calorimetry trace and a non-2-state model fit. The calculated Tm value was 49.38° C., AH was 6.159E4, and ΔHᵥ was 1.468E5. X axis=temperature (degrees C.); Y-axis=Cp (Kcal/mole/degrees C.).

Data: z008JC1dsc_cp
Model: MN2State
Chi\hat{ }2/DoF = 2.195E4

| | | |
|---|---|---|
| Tₘ | 49.38 | |
| ΔH | 6.159E4 | ±172 |
| ΔHᵥ | 1.468E5 | ±511 |

FIG. 5 is a DSC trace of DOM10-275-78 in potassium phosphate. The fitted data shows a calorimetry trace and a non-2-state model fit. The calculated Tm value was 49.77° C., ΔH was 5.975E4, and ΔHᵥ was 1.442E5. X axis=temperature (degrees C.); Y-axis=Cp (Kcal/mole/degrees C.).

Data: z011JC2dsc_cp
Model: MN2State
Chi\hat{ }2/DoF = 1.671E4

| | | |
|---|---|---|
| Tₘ | 49.77 | ±0.0066 |
| ΔH | 5.975E4 | ±152 |
| ΔHᵥ | 1.442E5 | ±455 |

FIG. 6 is a DSC trace for DOM10-53-474 in PBS. The fitted data shows a calorimetry trace and a non-2-state model fit. The calculated Tm value was 52.89° C., ΔH was 4.529E4, and ΔHᵥ was 1.354E5. X axis=temperature (degrees C.); Y-axis=Cp (Kcal/mole/degrees C.).

Data: z000810534742_cp
Model: MN2State
Chi\hat{ }2/DoF = 2.654E4

| | | |
|---|---|---|
| Tₘ | 52.89 | ±0.025 |
| ΔH | 4.529E4 | ±402 |
| ΔHᵥ | 1.354E5 | ±1.49E3 |

FIG. 7 is a graph showing the maximum solubility of DOM10-53-474 (open diamonds) and DOM10-275-78 (filled squares) in PBS. The experimental concentration was plotted against the theoretical concentration at that volume (dotted line) and the maximum solubility was taken as the point at which experimental concentration diverged from theoretical. The maximum solubility for both molecules exceeded 100 mg/ml. X axis=Theoretical concentration (mg/ml); Y axis=Actual concentration (mg/ml).

FIG. 8A-C is an SEC trace for DOM10-53-474 pre- (start material) and post nebulisation (aerosilized material) using a vibrating mesh nebuliser. The SEC profiles of the pre- (start material) and two post-nebulisation (aerosolized material) was identical. No peaks indicative of aggregation were seen post nebulisation.

FIG. 8D-F is an SEC trace for DOM10-53-474 pre- and post nebulisation using a jet nebuliser. The SEC profile of the pre- and two post-nebulisation were seen to be identical. No peaks indicative of aggregation were seen post nebulisation.

FIG. 9 is a table illustrating sandwich ELISA data for DOM10-53-474 pre- and post-nebulisation samples. The samples were analyzed for binding to human IL-13 and the potency was shown to be unaffected by nebulisation. Sample #14 represents 2.3 mg/ml, 25 mM sodium phosphate buffer pH 7.5, 7% (v/v) PEG1000, 1.2% (w/v) sucrose. Sample #15 represents 4.7 mg/mL 25 mM sodium phosphate buffer pH 7.5, 7% (v/v) PEG1000, 1.2% (w/v) sucrose. Sample #16 represents 2.6 mg/mL PBS. The material remaining in the cup after nebulisation is indicated by “CUP” and aerosolized material is indicated by “Aero”.

FIGS. 10A (normal) and 10B (zoom-in) are an SEC trace of DOM10-275-78 eluted from Protein A resin. The eluted protein was approximately 99% pure, containing approximately 1% of dimeric DOM10-275-78. The retention time was 22.46 minutes.

FIG. 11 is a chromatogram showing DOM10-275-78 on hydroxyapatite type II. The UV absorbance is shown by the solid line and the conductivity by the dotted line. The separation of both dimer and dAb-PrA complex from dAb monomer can be seen.

FIG. 12 is an SEC trace measuring the recovery of DOM10-275-78 after hydroxyapatite. The recovery was measured to be 74% based on absorbance at 280 nm and the purity was 100%. The retention time was 22.48 minutes.

FIG. 13 is a chromatogram showing the elution of DOM10-275-78 from a HIC phenyl column. The UV 280 trace is shown by the solid line and the conductivity by the dotted line.

### DETAILED DESCRIPTION OF THE INVENTION

Within this specification embodiments have been described in a way which enables a clear and concise specification to be written, but it is intended and will be appreciated that embodiments may be variously combined or separated without parting from the invention.

As used herein, the term “ligand” refers to a compound that comprises at least one peptide, polypeptide or protein moiety that has a binding site with binding specificity for a desired endogenous target compound (e.g., IL-4, IL-13). The ligands according to the invention in one embodiment comprise immunoglobulin variable domains which have different binding specificities, and do not contain variable domain pairs which together form a binding site for target compound (I.e., do not comprise an immunoglobulin heavy chain variable domain and an immunoglobulin light chain variable domain that together form a binding site for IL-4 or IL-13). In one embodiment each domain which has a binding site that has binding specificity for a target is an immunoglobulin single variable domain (e.g., immunoglobulin single heavy chain variable domain (e.g., V_{H}, V_{HH}), immunoglobulin single light chain variable domain (e.g., V_{L})) that has binding specificity for a desired target (e.g., IL-4, IL-13). Each polypeptide domain which has a binding site that has binding specificity for a target (e.g., IL-4, IL-13) can also comprise one or more complementarity determining regions (CDRs) of an antibody or antibody fragment (e.g., an immunoglobulin single variable domain) that has binding specificity for a desired target (e.g., IL-4, IL-13) in a suitable format, such that the binding domain has binding specificity for the target. For example, the CDRs can be grafted onto a suitable protein scaffold or skeleton, such as an affibody, a SpA scaffold, an LDL receptor class A domain, or an EGF domain. Further, the ligand can be bivalent (heterobivalent) or multivalent (heteromultivalent) as described herein. Thus, “ligands” include polypeptides that comprise two dAbs wherein each dAb binds to a different target (e.g., TL-4, IL-13). Ligands also include polypeptides that comprise at least two dAbs that bind different targets (or the CDRs of dAbs) in a suitable format, such as an antibody format (e.g., IgG-like format, scFv, Fab, Fab′, F(ab′)₂) or a suitable protein scaffold or skeleton, such as an affibody, a SpA scaffold, an LDL receptor class A domain, an EGF domain, avimer and multispecific ligands as described herein.

The polypeptide domain which has a binding site that has binding specificity for a target (e.g., IL-4, IL-13) can also be a protein domain comprising a binding site for a desired target, e.g., a protein domain is selected from an affibody, a SpA domain, an LDL receptor class A domain, an avimer (see, e.g., U.S. Patent Application Publication Nos. 2005/0053973, 2005/0089932, 2005/0164301). If desired, a “ligand” can further comprise one or more additional moieties, that can each independently be a peptide, polypeptide or protein moiety or a non-peptidic moiety (e.g., a polyalkylene glycol, a lipid, a carbohydrate). For example, the ligand can further comprise a half-life extending moiety as described herein (e.g., a polyalkylene glycol moiety, a moiety comprising albumin, an albumin fragment or albumin variant, a moiety comprising transferrin, a transferrin fragment or transferrin variant, a moiety that binds albumin, a moiety that binds neonatal Fc receptor).

As used herein, the phrase “target” refers to a biological molecule (e.g., peptide, polypeptide, protein, lipid, carbohydrate) to which a polypeptide domain which has a binding site can bind. The target can be, for example, an intracellular target (e.g., an intracellular protein target), a soluble target (e.g., a secreted protein such as IL-4, IL-13), or a cell surface target (e.g., a membrane protein, a receptor protein). In one embodiment, the target is IL-4 or IL-13.

The phrase “immunoglobulin single variable domain” refers to an antibody variable region (V_{H}, V_{HH}, V_{L}) that specifically binds a target, antigen or epitope independently of other V domains; however, as the term is used herein, an immunoglobulin single variable domain can be present in a format (e.g., hetero-multimer) with other variable regions or variable domains where the other regions or domains are not required for antigen binding by the single immunoglobulin variable domain (i.e., where the immunoglobulin single variable domain binds antigen independently of the additional variable domains). Each “immunoglobulin single variable domain” encompasses not only an isolated antibody single variable domain polypeptide, but also larger polypeptides that comprise one or more monomers of an antibody single variable domain polypeptide sequence. A “domain antibody” or “dAb” is the same as an “immunoglobulin single variable domain” polypeptide as the term is used herein. An immunoglobulin single variable domain polypeptide, is in one embodiment a mammalian immunoglobulin single variable domain polypeptide, an another embodiment human, and includes rodent immunoglobulin single variable domains (for example, as disclosed in WO 00/29004, the contents of which are incorporated herein by reference in their entirety) and camelid V_{HH} dAbs. As used herein, camelid dAbs are immunoglobulin single variable domain polypeptides which are derived from species including camel, llama, alpaca, dromedary, and guanaco, and comprise heavy chain antibodies naturally devoid of light chain (V_{HH}). Similar dAbs, can be obtained from single chain antibodies from other species, such as nurse shark. Possible ligands comprises at least two different immunoglobulin single variable domain polypeptides or at least two different dAbs. The immunoglobulin single variable domains (dAbs) described herein contain complementarity determining regions (CDR1, CDR2 and CDR3). The locations of CDRs and frame work (FR) regions and a numbering system have been defined by Kabat et al. (Kabat, E. A. et al., Sequences of Proteins of Immunological Interest, Fifth Edition, U.S. Department of Health and Human Services, U.S. Government Printing Office (1991)). The amino acid sequences of the CDRs (CDR1, CDR2, CDR3) of the V_{H} and V_{κ} dAbs disclosed herein will be readily apparent to the person of skill in the art based on the well known Kabat amino acid numbering system and definition of the CDRs. According to the Kabat numbering system V_{L} (V_{κ} or V_{λ}) CDR1 is from position 24-34, V_{L} CDR2 is from position 50-56, V_{L} CDR3 is from position 89-97, and V_{H} CDR1 is from position 31-35, V_{H} CDR2 is from position 50-65 and V_{H} CDR3 is from position 95-102. Heavy chain CDR-H3 have varying lengths, insertions are numbered between residue H100 and H101 with letters up to K (i.e. H100, H100A H100K, H101). Residue 103 which is the start of FR4 is almost always a W. CDRs can alternatively be determined using the system of Chothia (Chothia et al., (1989) Conformations of immunoglobulin hypervariable regions; Nature 342, p877-883), according to AbM or according to the Contact method as follows. See http://www.bioinf.org.uk/abs/ for suitable methods for determining CDRs.

Once each residue has been numbered, one can then apply the following CDR definitions (“-” means same residue numbers as shown for Kabat):

Kabat-most commonly used method based on sequence variability

(using Kabat numbering):
CDR H1: 31-35/35A/35B
CDR H2: 50-65
CDR H3: 95-102
CDR L1: 24-34
CDR L2: 50-56
CDR L3: 89-97

Chothia-based on location of the structural loop regions

(using Chothia numbering):
CDR H1: 26-32
CDR H2: 52-56
CDR H3: 95-102
CDR L1: 24-34
CDR L2: 50-56
CDR L3: 89-97

AbM-compromise between Kabat and Chothia

| (using Kabat numbering): | (using Chothia numbering): |
|---|---|
| CDR H1: 26-35/35A/35B | 26-35 |
| CDR H2: 50-58 | — |
| CDR H3: 95-102 | — |
| CDR L1: 24-34 | — |
| CDR L2: 50-56 | — |
| CDR L3: 89-97 | — |

Contact-based on crystal structures and prediction of
contact residues with antigen

| (using Kabat numbering): | (using Chothia numbering): |
|---|---|
| CDR H1: 30-35/35A/35B | 30-35 |
| CDR H2: 47-58 | — |
| CDR H3: 93-101 | — |
| CDR L1: 30-36 | — |
| CDR L2: 46-55 | — |
| CDR L3: 89-96 | — |

As used herein “interleukin-4” (IL-4) refers to naturally occurring or endogenous mammalian IL-4 proteins and to proteins having an amino acid sequence which is the same as that of a naturally occurring or endogenous corresponding mammalian IL-4 protein (e.g., recombinant proteins, synthetic proteins (i.e., produced using the methods of synthetic organic chemistry)). Accordingly, as defined herein, the term includes mature IL-4 protein, polymorphic or allelic variants, and other isoforms of an IL-4 and modified or unmodified forms of the foregoing (e.g., lipidated, glycosylated). Naturally occurring or endogenous IL-4 includes wild type proteins such as mature IL-4, polymorphic or allelic variants and other isoforms and mutant forms which occur naturally in mammals (e.g., humans, non-human primates). Such proteins can be recovered or isolated from a source which naturally produces IL-4, for example. These proteins and proteins having the same amino acid sequence as a naturally occurring or endogenous corresponding IL-4, are referred to by the name of the corresponding mammal. For example, where the corresponding mammal is a human, the protein is designated as a human IL-4. Several mutant IL-4 proteins are known in the art, such as those disclosed in WO 03/038041.

As used herein “interleukin-13” (IL-13) refers to naturally occurring or endogenous mammalian IL-13 proteins and to proteins having an amino acid sequence which is the same as that of a naturally occurring or endogenous corresponding mammalian IL-13 protein (e.g., recombinant proteins, synthetic proteins (i.e., produced using the methods of synthetic organic chemistry)). Accordingly, as defined herein, the term includes mature IL-13 protein, polymorphic or allelic variants, and other isoforms of IL-13 (e.g., produced by alternative splicing or other cellular processes), and modified or unmodified forms of the foregoing (e.g., lipidated, glycosylated). Naturally occurring or endogenous IL-13 include wild type proteins such as mature IL-13, polymorphic or allelic variants and other isoforms and mutant forms which occur naturally in mammals (e.g., humans, non-human primates). For example, as used herein IL-13 encompasses the human IL-13 variant in which Arg at position 110 of mature human IL-13 is replaced with Gln (position 110 of mature IL-13 corresponds to position 130 of the precursor protein) which is associed with asthma (atopic and nonatopic asthma) and other variants of IL-13. (Heinzmann et al., Hum Mol. Genet. 9:549-559 (2000).) Such proteins can be recovered or isolated from a source which naturally produces IL-13, for example. These proteins and proteins having the same amino acid sequence as a naturally occurring or endogenous corresponding IL-13, are referred to by the name of the corresponding mammal. For example, where the corresponding mammal is a human, the protein is designated as a human IL-13. Several mutant IL-13 proteins are known in the art, such as those disclosed in WO 03/035847.

“Affinity” and “avidity” are terms of art that describe the strength of a binding interaction. With respect to the ligands of the invention, avidity refers to the overall strength of binding between the targets (e.g., first cell surface target and second cell surface target) on the cell and the ligand. Avidity is more than the sum of the individual affinities for the individual targets.

As used herein, “toxin moiety” refers to a moiety that comprises a toxin. A toxin is an agent that has deleterious effects on or alters cellular physiology (e.g., causes cellular necrosis, apoptosis or inhibits cellular division).

As used herein, the term “dose” refers to the quantity of ligand administered to a subject all at one time (unit dose), or in two or more administrations over a defined time interval. For example, dose can refer to the quantity of ligand (e.g., ligand comprising an immunoglobulin single variable domain that binds IL-4 and an immunoglobulin single variable domain that binds IL-13) administered to a subject over the course of one day (24 hours) (daily dose), two days, one week, two weeks, three weeks or one or more months (e.g., by a single administration, or by two or more administrations). The interval between doses can be any desired amount of time.

As used herein “complementary” refers to when two immunoglobulin domains belong to families of structures which form cognate pairs or groups or are derived from such families and retain this feature. For example, a V_{H} domain and a V_{L} domain of an antibody are complementary; two V_{H} domains are not complementary, and two V_{L} domains are not complementary. Complementary domains may be found in other members of the immunoglobulin superfamily, such as the V_{α} and V_{β} (or γ and δ) domains of the T-cell receptor. Domains which are artificial, such as domains based on protein scaffolds which do not bind epitopes unless engineered to do so, are non-complementary. Likewise, two domains based on (for example) an immunoglobulin domain and a fibronectin domain are not complementary.

As used herein, “immunoglobulin” refers to a family of polypeptides which retain the immunoglobulin fold characteristic of antibody molecules, which contains two β sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signaling (for example, receptor molecules, such as the PDGF receptor). The present invention is applicable to all immunoglobulin superfamily molecules which possess binding domains. In one embodiment, the present invention relates to antibodies.

As used herein “domain” refers to a folded protein structure which retains its tertiary structure independently of the rest of the protein. Generally, domains are responsible for discrete functional properties of proteins, and in many cases may be added, removed or transferred to other proteins without loss of function of the remainder of the protein and/or of the domain. By single antibody variable domain is meant a folded polypeptide domain comprising sequences characteristic of antibody variable domains. It therefore includes complete antibody variable domains and modified variable domains, for example in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain. Thus, each ligand comprises at least two different domains.

“Repertoire” A collection of diverse variants, for example polypeptide variants which differ in their primary sequence. A library that encompasses a repertoire of polypeptides in one embodiment comprises at least 1000 members.

“Library” The term library refers to a mixture of heterogeneous polypeptides or nucleic acids. The library is composed of members, each of which have a single polypeptide or nucleic acid sequence. To this extent, library is synonymous with repertoire. Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. In one embodiment, each individual organism or cell contains only one or a limited number of library members. In one embodiment, the nucleic acids are incorporated into expression vectors, in order to allow expression of the polypeptides encoded by the nucleic acids. In a possible aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

As used herein an antibody refers to IgG, IgM, IgA, IgD or IgE or a fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria.

As described herein an “antigen’ is a molecule that is bound by a binding domain according to the present invention. Typically, antigens are bound by antibody ligands and are capable of raising an antibody response in vivo. It may be a polypeptide, protein, nucleic acid or other molecule. Generally, the dual-specific ligands according to the invention are selected for target specificity against two particular targets (e.g., antigens). In the case of conventional antibodies and fragments thereof, the antibody binding site defined by the variable loops (L1, L2, L3 and H1, H2, H3) is capable of binding to the antigen.

An “epitope” is a unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation.

“Universal framework” refers to a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, (1987) J. Mol. Biol. 196:910-917. The invention provides for the use of a single framework, or a set of such frameworks, which has been found to permit the derivation of virtually any binding specificity through variation in the hypervariable regions alone.

The phrase, “half-life,” refers to the time taken for the serum concentration of the ligand to reduce by 50%, in vivo, for example due to degradation of the ligand and/or clearance or sequestration of the dual-specific ligand by natural mechanisms. The ligands of the invention are stabilized in vivo and their half-life increased by binding to molecules which resist degradation and/or clearance or sequestration. Typically, such molecules are naturally occurring proteins which themselves have a long half-life in vivo. The half-life of a ligand is increased if its functional activity persists, in vivo, for a longer period than a similar ligand which is not specific for the half-life increasing molecule. Thus a ligand specific for HSA and two target molecules is compared with the same ligand wherein the specificity to HSA is not present, that is does not bind HSA but binds another molecule. For example, it may bind a third target on the cell. Typically, the half-life is increased by 10%, 20%, 30%, 40%, 50% or more. Increases in the range of 2×, 3×, 4×, 5×, 10×, 20×, 30×, 40×, 50× or more of the half-life are possible. Alternatively, or in addition, increases in the range of up to 30×, 40×, 50×, 60×, 70×, 80×, 90×, 100×, 150× of the half life are possible.

As referred to herein, the term “competes” means that the binding of a first target to its cognate target binding domain is inhibited when a second target is bound to its cognate target binding domain. For example, binding may be inhibited sterically, for example by physical blocking of a binding domain or by alteration of the structure or environment of a binding domain such that its affinity or avidity for a target is reduced.

As used herein, “epitopic specificity” refers to the fine specificity of an antigen binding moiety or domain, e.g., an antibody or antigen binding fragment thereof, such as a dAb, defined by the epitope that it binds, rather than the antigen that it binds. Two ligands (e.g. dAbs) that have the same epitopic specificity bind to the same epitope.

As used herein, the term “inhibits” means to reduce and or prevent (i.e., both partial or complete inhibition is encompassed). For example, a dAb may prevent binding of a ligand (e.g., a different dAb) to its target, or inhibit binding of a ligand (e.g., a different dAb) to its target by at least about 25%, at least about 50%, at least about 60%, at least about 70%, at least about 80%, at least about 85%, at least about 90%, or at least about 95%.

As used herein, the terms “low stringency,” “medium stringency,” “high stringency,” or “very high stringency conditions” describe conditions for nucleic acid hybridization and washing. Guidance for performing hybridization reactions can be found in Current Protocols in Molecular Biology, John Wiley & Sons, N.Y. (1989), 6.3.1-6.3.6, which is incorporated herein by reference in its entirety. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and in one embodiment (4) very high stringency hybridization conditions are 0.5M sodium phosphate, 7% SDS at 65° C., followed by one or more washes at 0.2×SSC, 1% SDS at 65° C. Very high stringency conditions (4) are the possible conditions and the ones that should be used unless otherwise specified.

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

Calculations of “homology” or “sequence identity” or “similarity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a possible embodiment, the length of a reference sequence aligned for comparison purposes is at least 30%, in one embodiment at least 40%, at least 50%, at least 60%, at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences.

Amino acid and nucleotide sequence alignments and homology, similarity or identity, as defined herein are in one embodiment prepared and determined using the algorithm BLAST 2 Sequences, using default parameters (Tatusova, T. A. et al., FEMS Microbiol Lett, 174:187-188 (1999)). Alternatively, the BLAST algorithm (version 2.0) is employed for sequence alignment, with parameters set to default values. BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, 1990, Proc. Natl. Acad. Sci. USA 87(6):2264-8.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc. which are incorporated herein by reference) and chemical methods.

The invention relates to ligands that have binding specificity for IL-13 (e.g., human IL-13), and to ligands that have binding specificity for IL-4 and IL-13 (e.g., human IL-4 and human IL-13). For example, the ligand can comprise a polypeptide domain having a binding site with binding specificity for IL-13, or comprise a polypeptide domain having a binding site with binding specificity for IL-4 and a polypeptide domain having a binding site with binding specificity for IL-13.

The invention also relates to ligands that have cross-reactivity with human IL-4 and a non-human IL-4 (e.g., rhesus IL-4, cynomolgous IL-4), ligands that have cross-reactivity with human IL-13 and a non-human IL-13 (e.g., rhesus IL-13, cynomolgous IL-13), and to ligands that have binding specificity for human IL-4, human IL-13, non-human IL-4 and non-human IL-13 (e.g., rhesus IL-4, rhesus IL-13, cynomolgous IL-4 and cynomolgous IL-13).

The ligands of the invention provide several advantages. For example, as described herein, the ligand can be tailored to have a desired in vivo serum half-life. Domain antibodies are much smaller than conventional antibodies, and can be administered to achieve better tissue penetration than conventional antibodies. Thus, dAbs and ligands that comprise a dAb provide advantages over conventional antibodies when administered to treat disease, such as Th2-mediated disease, asthma, allergic diseases, cancer (e.g., renal cell cancer). For example, asthma (e.g. allergic asthma) can be IgE-mediated or non-IgE-mediated, and ligands that have binding specificity for IL-13 or IL-4 and IL-13 can be administered to treat both IgE-mediated and non-IgE-mediated asthma.

Similarly, due to the overlap and similarity in the biological activity of IL-4 and IL-13, therapy with agents that bind and inhibit only one of these cytokines may not produce the desired effects in all circumstances. Accordingly, ligands that have binding specificity for IL-4 and IL-13 can be administered to a patient (e.g., a patient with allergic disease (e.g., allergic asthma)) to provide superior therapy using a single therapeutic agent.

In some embodiments, the ligand has binding specificity for IL-13 and comprises an (at least one) immunoglobulin single variable domain with binding specificity for IL-13. In certain embodiments, the ligand has binding specificity for IL-4 and IL-13, and comprises an (at least one) immunoglobulin single variable domain with binding specificity for IL-4 and an (at least one) immunoglobulin single variable domain with binding specificity for IL-13.

The ligand of the invention can be formatted as described herein. For example, the ligand of the invention can be formatted to tailor in vivo serum half-life. If desired, the ligand can further comprise a toxin or a toxin moiety as described herein. In some embodiments, the ligand comprises a surface active toxin, such as a free radical generator (e.g., selenium containing toxin) or a radionuclide. In other embodiments, the toxin or toxin moiety is a polypeptide domain (e.g., a dAb) having a binding site with binding specificity for an intracellular target. In particular embodiments, the ligand is an IgG-like format that has binding specificity for IL-4 and IL-13 (e.g., human IL-4 and human IL-13).

In one aspect, the invention relates to a ligand that has binding specificity for interleukin-4 (IL-4) and interleukin-13 (IL-13) comprising a protein moiety that has a binding site with binding specificity for IL-4; and a protein moiety that has a binding site with binding specificity for IL-13. The ligand that has binding specificity for IL-4 and IL-13 of this aspect of the invention, can be further characterized by any one or any combination of the following: (1) the proviso that said protein moiety that has a binding site with binding specificity for IL-4 is not an IL-4 receptor or IL-4-binding portion thereof, and said protein moiety that has a binding site with binding specificity for IL-13 is not an IL-13 receptor or IL-13-binding portion thereof; (2) the proviso that said binding site with binding specificity for TL-4 and said binding site with binding specificity for IL-13 each consist of a single amino acid chain; (3) the proviso that neither said binding site with binding specificity for IL-4 nor said binding site with binding specificity for IL-13 comprise an immunoglobulin heavy chain variable domain and an immunoglobulin light chain variable domain; and (4) the proviso that said protein moiety that has a binding site with binding specificity for IL-4 is not an antibody that binds IL-4 or an antigen-binding fragment thereof that comprises an immunoglobulin heavy chain variable domain and an immunoglobulin light chain variable domain that together form a binding site for IL-4, and said protein moiety that has a binding site with binding specificity for IL-13 is not an antibody that binds IL-13 or an antigen-binding fragment thereof that comprises an immunoglobulin heavy chain variable domain and an immunoglobulin light chain variable domain that together form a binding site for IL-13.

In one aspect, the invention relates to a ligand that has binding specificity for IL-13, comprising a protein moiety that has a binding site with binding specificity for IL-13. The ligand that has binding specificity for IL-13 of this aspect of the invention, can be further characterized by any one or any combination of the following: (1) the proviso that said protein moiety that has a binding site with binding specificity for IL-13 is not an antibody that binds IL-13 or an antigen-binding fragment thereof that comprises an immunoglobulin heavy chain variable domain and an immunoglobulin light chain variable domain that together form a binding site for IL-13; (2) the proviso that said protein moiety that has a binding site with binding specificity for IL-13 is not an IL-13 receptor or IL-13-binding portion thereof; (3) the proviso that said binding site with binding specificity for IL-13 consists of a single amino acid chain; and (4) the proviso that said binding site with binding specificity for IL-13 does not consist of an immunoglobulin heavy chain variable domain and an immunoglobulin light chain variable domain.

### Ligand Formats

The ligand of the invention can be formatted as a monospecific, dual specific or multispecific ligand as described herein. See, also WO 03/002609, the entire teachings of which are incorporated herein by reference, regarding ligand formatting. Such dual specific ligands comprise immunoglobulin single variable domains that have different binding specificities. Such dual specific ligands can comprise combinations of heavy and light chain domains. For example, the dual specific ligand may comprise a V_{H} domain and a V_{L} domain, which may be linked together in the form of an scFv (e.g., using a suitable linker such as Gly₄Ser), or formatted into a bispecific antibody or antigen-binding fragment theref (e.g. F(ab′)₂, Fab′, Fab fragment). The dual specific ligands do not comprise complementary V_{H}/V_{L} pairs which form a conventional two chain antibody antigen-binding site that binds antigen or epitope co-operatively. Instead, the dual format ligands can comprise a V_{H}/V_{L} complementary pair, wherein the V domains have different binding specificities.

The ligand (e.g., monospecific, dual specific ligands) may comprise one or more C_{H} or C_{L} domains if desired. A hinge region domain may also be included if desired. Such combinations of domains may, for example, mimic natural antibodies, such as IgG or IgM, or fragments thereof, such as Fv, scFv, Fab or F(ab′)₂ molecules. Other structures, such as a single arm of an IgG molecule comprising V_{H}, V_{L}, C_{H}1 and C_{L} domains, are envisaged. The ligand can comprise a heavy chain constant region of an immunoglobulin (e.g., IgG (e.g., IgG1, IgG2, IgG3, IgG4) IgM, IgA, IgD or IgE) or portion thereof (e.g., Fc portion) and/or a light chain constant region (e.g., C_{λ}, C_{κ}). For example, the ligand can comprise CH1 of IgG1 (e.g., human IgG1), CH1 and CH2 of IgG1 (e.g., human IgG1), CH1, CH2 and CH3 of IgG1 (e.g., human IgG1), CH2 and CH3 of IgG1 (e.g., human IgG1), or CH1 and CH3 of IgG1 (e.g., human IgG1).

In one example, a dual specific ligand of the invention comprises only two variable domains although several such ligands may be incorporated together into the same protein, for example two such ligands can be incorporated into an IgG or a multimeric immunoglobulin, such as IgM. Alternatively, in another embodiment a plurality of dual specific ligands are combined to form a multimer. For example, two different dual specific ligands are combined to create a tetra-specific molecule. It will be appreciated by one skilled in the art that the light and heavy variable regions of a dual-specific ligand of the present invention may be on the same polypeptide chain, or alternatively, on different polypeptide chains. In the case that the variable regions are on different polypeptide chains, then they may be linked via a linker, generally a flexible linker (such as a polypeptide chain), a chemical linking group, or any other method known in the art.

Ligands can be formatted as bi- or multispecific antibodies or antibody fragments or into bi- or multispecific non-antibody structures. Suitable formats include, any suitable polypeptide structure in which an antibody variable domain or one or more of the CDRs thereof can be incorporated so as to confer binding specificity for antigen on the structure. A variety of suitable antibody formats are known in the art, such as, bispecific IgG-like formats (e.g., chimeric antibodies, humanized antibodies, human antibodies, single chain antibodies, heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single variable domain (e.g., V_{H}, V_{L}, V_{HH}), a dAb, and modified versions of any of the foregoing (e.g., modified by the covalent attachment of polyalkylene glycol (e.g., polyethylene glycol, polypropylene glycol, polybutylene glycol) or other suitable polymer). See, PCT/GB03/002804, filed Jun. 30, 2003, which designated the United States, (WO 2004/081026) regarding PEGylated of single variable domains and dAbs, suitable methods for preparing same, increased in vivo half life of the PEGylated single variable domains and dAb monomers and multimers, suitable PEGs, possible hydrodynamic sizes of PEGs, and possible hydrodynamic sizes of PEGylated single variable domains and dAb monomers and multimers. The entire teaching of PCT/GB03/002804 (WO 2004/081026), including the portions referred to above, are incorporated herein by reference.

The ligand can be formatted using a suitable linker such as (Gly₄Ser)ₙ, where n=from 1 to 8, (e.g., 1, 2, 3, 4, 5, 6 or 7). If desired, ligands, including dAb monomers, dimers and trimers, can be linked to an antibody Fc region, comprising one or both of C_{H}2 and C_{H}3 domains, and optionally a hinge region. For example, vectors encoding ligands linked as a single nucleotide sequence to an Fc region may be used to prepare such polypeptides.

Ligands and dAb monomers can also be combined and/or formatted into non-antibody multi-ligand structures to form multivalent complexes, which bind target molecules with the same antigen, thereby providing superior avidity. For example natural bacterial receptors such as SpA can been used as scaffolds for the grafting of CDRs to generate ligands which bind specifically to one or more epitopes. Details of this procedure are described in U.S. Pat. No. 5,831,012. Other suitable scaffolds include those based on fibronectin and affibodies. Details of suitable procedures are described in WO 98/58965. Other suitable scaffolds include lipocallin and CTLA4, as described in van den Beuken et al., J. Mol. Biol. 310:591-601 (2001), and scaffolds such as those described in WO 00/69907 (Medical Research Council), which are based for example on the ring structure of bacterial GroEL or other chaperone polypeptides. Protein scaffolds may be combined; for example, CDRs may be grafted on to a CTLA4 scaffold and used together with immunoglobulin V_{H} or V_{L} domains to form a ligand. Likewise, fibronectin, lipocallin and other scaffolds may be combined

A variety of suitable methods for preparing any desired format are known in the art. For example, antibody chains and formats (e.g., monospecific, bispecific, trispecific or tetraspecific IgG-like formats, chimeric antibodies, humanized antibodies, human antibodies, single chain antibodies, homodimers and heterodimers of antibody heavy chains and/or light chains) can be prepared by expression of suitable expression constructs and/or culture of suitable cells (e.g., hybridomas, heterohybridomas, recombinant host cells containing recombinant constructs encoding the format). Further, formats such as antigen-binding fragments of antibodies or antibody chains (e.g., bispecific binding fragments, such as a Fv fragment (e.g., single chain Fv (scFv), a disulfide bonded Fv), a Fab fragment, a Fab′ fragment, a F(ab′)₂ fragment), can be prepared by expression of suitable expression constructs or by enzymatic digestion of antibodies, for example using papain or pepsin.

The ligand can be formatted as a multispecific ligand, for example as described in WO 03/002609, the entire teachings of which are incorporated herein by reference. Such multispecific ligand possesses more than one epitope binding specificity. Generally, the multi-specific ligand comprises two or more epitope binding domains, such dAbs or non-antibody protein domain comprising a binding site for an epitope, e.g., an affibody, a SpA domain, an LDL receptor class A domain, an EGF domain, an avimer. Multispecific ligands can be formatted further as described herein.

In some embodiments, the ligand is an IgG-like format. Such formats have the conventional four chain structure of an IgG molecule (2 heavy chains and two light chains), in which one or more of the variable regions (V_{H} and or V_{L}) have been replaced with a dAb or immunoglobulin single variable domain of a desired specificity. In one embodiment, each of the variable regions (2 V_{H} regions and 2 V_{L} regions) is replaced with a dAb or immunoglobulin single variable domain. The dAb(s) or immunoglobulin single variable domain(s) that are included in an IgG-like format can have the same specificity or different specificities. In some embodiments, the IgG-like format is tetravalent and can have one, two, three or four specificities. The IgG-like format can be bispecific and comprise, for example, a first and second dAb that have the same specificity, a third dAb with a different specificity and a fourth dAb with a different specificity from the first, second and third dAbs; or tetraspecific and comprise four dAbs that each have a different specificity.

The IgG-like format can be monospecific and comprise 4 dAbs that have the specificity for IL-4 or for IL-13. The IgG-like format can be bispecific and comprise, for example, 3 dAbs that have specificity for IL-4 and another dAb that has specificity for IL-13, or bispecific and comprise, for example two dAbs that have specificity for IL-4 and two dAbs that have specificity for IL-13. The IgG-like format can be bispecific and comprise, for example, 3 dAbs that have specificity for IL-13 and another dAb that has specificity for IL-14. When the IgG-like format contains two or more dAbs that bind IL-4, the dAbs can bind to the same or different epitopes. For example, the IgG-like format can comprise two, three or four dAbs that have binding specificity for IL-4 that bind the same or different epitopes on IL-4. Similarly, when the IgG-like format contains two or more dAbs that bind IL-13, the dAbs can bind to the same or different epitopes. For example, the IgG-like format can comprise two, three or four dAbs that have binding specificity for IL-13 that bind the same or different epitopes on IL-13.

In one example, the IgG-like format is a tetravalent IgG-like ligand that has binding specificity for IL-4 or IL-13 comprising two heavy chains and two light chains, wherein said heavy chains comprise the constant region of an immunoglobulin heavy chain and a single immunoglobulin variable domain that has binding specificity for IL-4 or IL-13; and said light chains comprise the constant region of an immunoglobulin light chain and a single immunoglobulin variable domain that has binding specificity for IL-4 or IL-13. The IgG-like format of this example can be further characterized by the proviso that when said heavy chains comprise a single immunoglobulin variable domain that has binding specificity for IL-4, said light chains comprise a single immunoglobulin variable domain that has binding specificity for IL-13; and when said heavy chains comprise a single immunoglobulin variable domain that has binding specificity for IL-13, said light chains comprise a single immunoglobulin variable domain that has binding specificity for IL-4.

Antigen-binding fragments of IgG-like formats (e.g., Fab, F(ab′)₂, Fab′, Fv, scFᵥ) can be prepared. In addition, a particular constant region or Fc portion (e.g., constant region or Fc portion of an IgG, such as IgG1 (e.g., CH1, CH2 and CH3; CH2 and CH3)), variant or portion thereof can be selected in order to tailor effector function. For example, if complement activation and/or antibody dependent cellular cytotoxicity (ADCC) function is desired, the ligand can be an IgG1-like format. If desired, the IgG-like format can comprise a mutated constant region (variant IgG heavy chain constant region) to minimize binding to Fc receptors and/or ability to fix complement. (see e.g. Winter et al, GB 2,209,757 B; Morrison et al., WO 89/07142; Morgan et al., WO 94/29351, Dec. 22, 1994).

The ligands of the invention can be formatted as a fusion protein that contains a first immunoglobulin single variable domain that is fused directly (e.g., through a peptide bond) or through a suitable linker (amino acid, peptide, polypeptide) to a second immunoglobulin single variable domain. If desired such a format can further comprise, for example, one or more immunoglobulin domains (e.g., constant region, Fc portion) and/or a half life extending moiety as described herein. For example, the ligand can comprise a first immunoglobulin single variable domain that is fused directly to a second immunoglobulin single variable domain that is fused directly to an immunoglobulin single variable domain that binds serum albumin.

In one example, the ligand comprises a first single immunoglobulin single variable domain, a second immunoglobulin single variable domain and an Fc portion or an immunoglobulin constant region. The first and second immunoglobulin single variable domains can each have binding specificity for IL-4 or IL-13. Accordingly, this type of ligand can contain two binding sites (be bivalent) wherein each bindng site binds IL-13 or wherein one binding site binds IL-4 and one binding site binds IL-13. For example, the ligands can have the structure V domain-V domain-IgG constant region or V domain-V domain-IgG Fc portion.

Generally the orientation of the polypeptide domains that have a binding site with binding specificity for a target and whether the ligand comprises a linker is a matter of design choice. However, some orientations, with or without linkers, may provide better binding characteristics than other orientations. All orientations (e.g., dAb1-linker-dAb2; dAb2-linker-dAb1) are encompassed by the invention, and ligands that contain an orientation that provides desired binding characteristics can be easily identified by screening.

### Half-Life Extended Formats

The ligand, and dAb monomers disclosed herein, can be formatted to extend its in vivo serum half life. Increased in vivo half-life is useful in in vivo applications of immunoglobulins, especially antibodies and most especially antibody fragments of small size such as dAbs. Such fragments (Fvs, disulphide bonded Fvs, Fabs, scFvs, dAbs) are rapidly cleared from the body, which can limit clinical applications.

A ligand can be formatted as a larger antigen-binding fragment of an antibody or as an antibody (e.g., formatted as a Fab, Fab′, F(ab)₂, F(ab′)₂, IgG, scFv) that has larger hydrodynamic size. Ligands can also be formatted to have a larger hydrodynamic size, for example, by attachment of a polyalkyleneglycol group (e.g. polyethyleneglycol (PEG) group, polypropylene glycol, polybutylene glycol), serum albumin, transferrin, transferrin receptor or at least the transferrin-binding portion thereof, an antibody Fc region, or by conjugation to an antibody domain. In some embodiments, the ligand (e.g., dAb monomer) is PEGylated. In one embodiment the PEGylated ligand (e.g., dAb monomer) binds IL-4 and/or IL-13 with substantially the same affinity or avidity as the same ligand that is not PEGylated. For example, the ligand can be a PEGylated ligand comprising a dAb that binds IL-4 or IL-13 with an affinity or avidity that differs from the avidity of ligand in unPEGylated form by no more than a factor of about 1000, in one embodiment no more than a factor of about 100, or no more than a factor of about 10, or with affinity or avidity substantially unchanged relative to the unPEGylated form. See, PCT/GB03/002804, filed Jun. 30, 2003, which designated the United States, (WO 2004/081026) regarding PEGylated single variable domains and dAbs, suitable methods for preparing same, increased in vivo half-life of the PEGylated single variable domains and dAb monomers and multimers, suitable PEGs, possible hydrodynamic sizes of PEGs, and possible hydrodynamic sizes of PEGylated single variable domains and dAb monomers and multimers. The entire teaching of PCT/GB03/002804 (WO 2004/081026), including the portions referred to above, are incorporated herein by reference.

Hydrodynamic size of the ligands (e.g., dAb monomers and multimers) of the invention may be determined using methods which are well known in the art. For example, gel filtration chromatography may be used to determine the hydrodynamic size of a ligand. Suitable gel filtration matrices for determining the hydrodynamic sizes of ligands, such as cross-linked agarose matrices, are well known and readily available.

The size of a ligand format (e.g., the size of a PEG moiety attached to a dAb monomer), can be varied depending on the desired application. For example, where a ligand is intended to leave the circulation and enter into peripheral tissues, it is desirable to keep the hydrodynamic size of the ligand low to facilitate diffusion from the blood stream. Alternatively, where it is desired to have the ligand remain in the systemic circulation for a longer period of time the size of the ligand can be increased, for example by formatting as an IgG-like protein or by addition of a 30 to 60 kDa PEG moiety (e.g., linear or branched 30 kDa PEG to 40 kDa PEG, such as addition of two 20 kDa PEG moieties.) The size of the ligand format can be tailored to achieve a desired in vivo serum half-life. For example, the size of the ligand format can be tailored to control exposure to a toxin and/or to reduce side effects of toxic agents.

The hydrodynamic size of a ligand (e.g., dAb monomer) and its serum half-life can also be increased by conjugating or linking the ligand to a binding domain (e.g., antibody or antibody fragment) that binds an antigen or epitope that increases half-life in vivo, as described herein. For example, the ligand (e.g., dAb monomer) can be conjugated or linked to an anti-serum albumin or anti-neonatal Fc receptor antibody or antibody fragment, (e.g., an anti-SA or anti-neonatal Fc receptor dAb, Fab, Fab′ or scFv), or to an anti-SA affibody or anti-neonatal Fc receptor affibody.

Examples of suitable albumin, albumin fragments or albumin variants for use in a ligand according to the invention are described in WO 2005/077042A2, which is incorporated herein by reference in its entirety. In particular, the following albumin, albumin fragments or albumin variants can be used in the present invention:
SEQ ID NO:1 (as disclosed in WO 2005/077042A2, this sequence being explicitly incorporated into the present disclosure by reference);
Albumin fragment or variant comprising or consisting of amino acids 1-387 of SEQ ID NO:1 in WO 2005/077042A2;
Albumin, or fragment or variant thereof, comprising an amino acid sequence selected from the group consisting of: (a) amino acids 54 to 61 of SEQ ID NO:1 in WO 20051077042A2; (b) amino acids 76 to 89 of SEQ ID NO:1 in WO 2005/077042A2; (c) amino acids 92 to 100 of SEQ ID NO:1 in WO 2005/077042A2: (d) amino acids 170 to 176 of SEQ ID NO:1 in WO 2005/077042A2; (e) amino acids 247 to 252 of SEQ ID NO:1 in WO 2005/077042A2: (f) amino acids 266 to 277 of SEQ ID NO:1 in WO 2005/077042A2; (g) amino acids 280 to 288 of SEQ ID NO:1 in WO 2005/077042A2; (h) amino acids 362 to 368 of SEQ ID NO:1 in WO 2005/077042A2; (i) amino acids 439 to 447 of SEQ ID NO:1 in WO 2005/077042A2 (j) amino acids 462 to 475 of SEQ ID NO:1 in WO 2005/077042A2; (k) amino acids 478 to 486 of SEQ ID NO:1 in WO 2005/077042A2; and (l) amino acids 560 to 566 of SEQ ID NO:1 in WO 2005/077042A2.

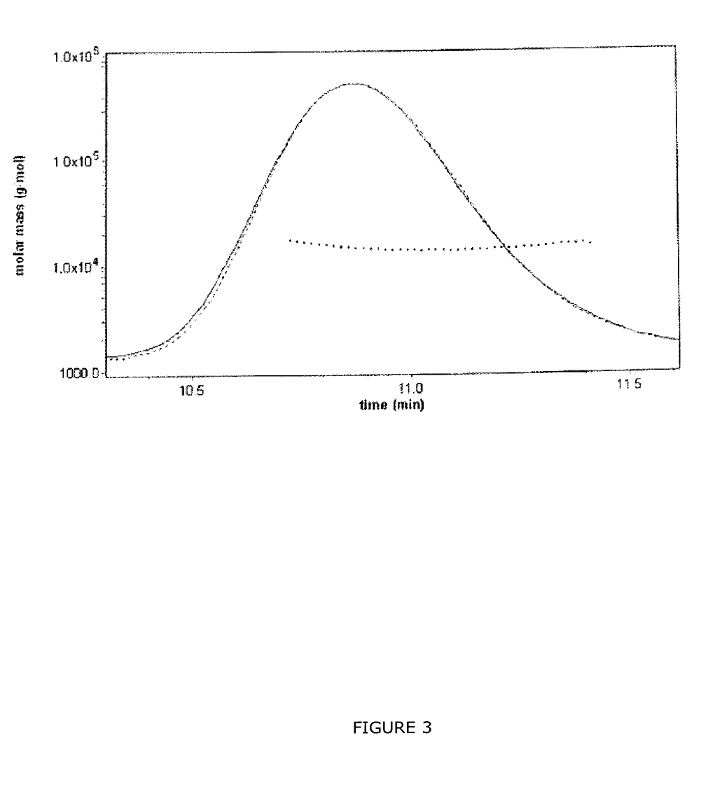

Further examples of suitable albumin, fragments and analogs for use in a ligand according to the invention are described in WO 03/076567A2, which is incorporated herein by reference in its entirety. In particular, the following albumin, fragments or variants can be used in the present invention:
Human serum albumin as described in WO 03/076567A2, (e.g., in FIG. 3) (this sequence information being explicitly incorporated into the present disclosure by reference);
Human serum albumin (HA) consisting of a single non-glycosylated polypeptide chain of 585 amino acids with a formula molecular weight of 66,500 (See, Meloun, et al., FEBS Letters 58:136 (1975); Behrens, et al., Fed. Proc. 34:591 (1975); Lawn, et al., Nucleic Acids Research 9:6102-6114 (1981); Minghetti, et al., J. Biol. Chem. 261:6747 (1986));
A polymorphic variant or analog or fragment of albumin as described in Weitkamp, et al., Ann. Hum. Genet. 37:219 (1973);
An albumin fragment or variant as described in EP 322094, (e.g., HA(1-373), HA(1-388), HA(1-389), HA(1-369), and HA(1-419) and fragments between 1-369 and 1-419);
An albumin fragment or variant as described in EP 399666, (e.g., HA(1-177) and HA(1-200) and fragments between HA(1-X), where X is any number from 178 to 199).

Where a (one or more) half-life extending moiety (e.g., albumin, transferrin and fragments and analogs thereof) is used in the ligands of the invention, it can be conjugated to the ligand using any suitable method, such as, by direct fusion to the target-binding moiety (e.g., dAb or antibody fragment), for example by using a single nucleotide construct that encodes a fusion protein, wherein the fusion protein is encoded as a single polypeptide chain with the half-life extending moiety located N- or C-terminally to the cell surface target binding moieties. Alternatively, conjugation can be achieved by using a peptide linker between moieties, (e.g., a peptide linker as described in WO 03/076567A2 or WO 2004/003019) (these linker disclosures being incorporated by reference in the present disclosure to provide examples for use in the present invention).

Typically, a polypeptide that enhances serum half-life in vivo is a polypeptide which occurs naturally in vivo and which resists degradation or removal by endogenous mechanisms which remove unwanted material from the organism (e.g., human). For example, a polypeptide that enhances serum half-life in vivo can be selected from proteins from the extracellular matrix, proteins found in blood, proteins found at the blood brain barrier or in neural tissue, proteins localized to the kidney, liver, lung, heart, skin or bone, stress proteins, disease-specific proteins, or proteins involved in Fc transport.

Suitable polypeptides that enhance serum half-life in vivo include, for example, transferrin receptor specific ligand-neuropharmaceutical agent fusion proteins (see U.S. Pat. No. 5,977,307, the teachings of which are incorporated herein by reference), brain capillary endothelial cell receptor, transferrin, transferrin receptor (e.g., soluble transferrin receptor), insulin, insulin-like growth factor 1 (IGF 1) receptor, insulin-like growth factor 2 (IGF 2) receptor, insulin receptor, blood coagulation factor X, α1-antitrypsin and HNF 1α. Suitable polypeptides that enhance serum half-life also include alpha-1 glycoprotein (orosomucoid; AAG), alpha-1 antichymotrypsin (ACT), alpha-1 microglobulin (protein HC; AIM), antithrombin III (AT III), apolipoprotein A-1 (Apo A-1), apolipoprotein B (Apo B), ceruloplasmin (Cp), complement component C3 (C3), complement component C4 (C4), C1 esterase inhibitor (C1 INH), C-reactive protein (CRP), ferritin (FER), hemopexin (HPX), lipoprotein(a) (Lp(a)), mannose-binding protein (MBP), myoglobin (Myo), prealbumin (transthyretin; PAL), retinol-binding protein (RBP), and rheumatoid factor (RF).

Suitable proteins from the extracellular matrix include, for example, collagens, laminins, integrins and fibronectin. Collagens are the major proteins of the extracellular matrix. About 15 types of collagen molecules are currently known, found in different parts of the body, e.g. type I collagen (accounting for 90% of body collagen) found in bone, skin, tendon, ligaments, cornea, internal organs or type II collagen found in cartilage, vertebral disc, notochord, and vitreous humor of the eye.

Suitable proteins from the blood include, for example, plasma proteins (e.g., fibrin, α-2 macroglobulin, serum albumin, fibrinogen (e.g., fibrinogen A, fibrinogen B), serum amyloid protein A, haptoglobin, profilin, ubiquitin, uteroglobulin and β-2-microglobulin), enzymes and enzyme inhibitors (e.g., plasminogen, lysozyme, cystatin C, alpha-1-antitrypsin and pancreatic trypsin inhibitor), proteins of the immune system, such as immunoglobulin proteins (e.g., IgA, IgD, IgE, IgG, IgM, immunoglobulin light chains (kappa/lambda)), transport proteins (e.g., retinol binding protein, α-1 microglobulin), defensins (e.g., beta-defensin 1, neutrophil defensin 1, neutrophil defensin 2 and neutrophil defensin 3) and the like.

Suitable proteins found at the blood brain barrier or in neural tissue include, for example, melanocortin receptor, myelin, ascorbate transporter and the like.

Suitable polypeptides that enhance serum half-life in vivo also include proteins localized to the kidney (e.g., polycystin, type IV collagen, organic anion transporter KI, Heymann's antigen), proteins localized to the liver (e.g., alcohol dehydrogenase, G250), proteins localized to the lung (e.g., secretory component, which binds IgA), proteins localized to the heart (e.g., HSP 27, which is associated with dilated cardiomyopathy), proteins localized to the skin (e.g., keratin), bone specific proteins such as morphogenic proteins (BMPs), which are a subset of the transforming growth factor β superfamily of proteins that demonstrate osteogenic activity (e.g., BMP-2, BMP-4, BMP-5, BMP-6, BMP-7, BMP-8), tumor specific proteins (e.g., trophoblast antigen, herceptin receptor, oestrogen receptor, cathepsins (e.g., cathepsin B, which can be found in liver and spleen)).

Suitable disease-specific proteins include, for example, antigens expressed only on activated T-cells, including LAG-3 (lymphocyte activation gene), osteoprotegerin ligand (OPGL; see Nature 402, 304-309 (1999)), OX40 (a member of the TNF receptor family, expressed on activated T cells and specifically up-regulated in human T cell leukemia virus type-I (HTLV-I)-producing cells; see Immunol. 165 (1):263-70 (2000)). Suitable disease-specific proteins also include, for example, metalloproteases (associated with arthritis/cancers) including CG6512 Drosophila, human paraplegin, human FtsH, human AFG3L2, murine ftsH; and angiogenic growth factors, including acidic fibroblast growth factor (FGF-1), basic fibroblast growth factor (FGF-2), vascular endothelial growth factor/vascular permeability factor (VEGF/VPF), transforming growth factor-alpha (TGF-α), tumor necrosis factor-alpha (TNF-α), angiogenin, interleukin-3 (IL-3), interleukin-8 (IL-8), platelet-derived endothelial growth factor (PD-ECGF), placental growth factor (P1GF), midkine platelet-derived growth factor-BB (PDGF), and fractalkine.

Suitable polypeptides that enhance serum half-life in vivo also include stress proteins such as heat shock proteins (HSPs). HSPs are normally found intracellularly. When they are found extracellularly, it is an indicator that a cell has died and spilled out its contents. This unprogrammed cell death (necrosis) occurs when as a result of trauma, disease or injury, extracellular HSPs trigger a response from the immune system. Binding to extracellular HSP can result in localizing the compositions of the invention to a disease site.

Suitable proteins involved in Fc transport include, for example, Brambell receptor (also known as FcRB). This Fc receptor has two functions, both of which are potentially useful for delivery. The functions are (1) transport of IgG from mother to child across the placenta (2) protection of IgG from degradation thereby prolonging its serum half-life. It is thought that the receptor recycles IgG from endosomes. (See, Holliger et al, Nat Biotechnol 15(7):632-6 (1997).)

Methods for pharmacokinetic analysis and determination of ligand half-life will be familiar to those skilled in the art. Details may be found in Kenneth, A. et al: Chemical Stability of Pharmaceuticals: A Handbook for Pharmacists and in Peters et al, Pharmacokinetic Analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half lives and area under the curve (AUC).

Ligands that Contain a Toxin Moiety or Toxin

The invention also relates to ligands that comprise a toxin moiety or toxin. Suitable toxin moieties comprise a toxin (e.g., surface active toxin, cytotoxin). The toxin moiety or toxin can be linked or conjugated to the ligand using any suitable method. For example, the toxin moiety or toxin can be covalently bonded to the ligand directly or through a suitable linker. Suitable linkers can include noncleavable or cleavable linkers, for example, pH cleavable linkers that comprise a cleavage site for a cellular enzyme (e.g., cellular esterases, cellular proteases such as cathepsin B). Such cleavable linkers can be used to prepare a ligand that can release a toxin moiety or toxin after the ligand is internalized.

A variety of methods for linking or conjugating a toxin moiety or toxin to a ligand can be used. The particular method selected will depend on the toxin moiety or toxin and ligand to be linked or conjugated. If desired, linkers that contain terminal functional groups can be used to link the ligand and toxin moiety or toxin. Generally, conjugation is accomplished by reacting toxin moiety or toxin that contains a reactive functional group (or is modified to contain a reactive functional group) with a linker or directly with a ligand. Covalent bonds formed by reacting a toxin moiety or toxin that contains (or is modified to contain) a chemical moiety or functional group that can, under appropriate conditions, react with a second chemical group thereby forming a covalent bond. If desired, a suitable reactive chemical group can be added to ligand or to a linker using any suitable method. (See, e.g., Hermanson, G. T., Bioconjugate Techniques, Academic Press: San Diego, Calif. (1996).) Many suitable reactive chemical group combinations are known in the art, for example an amine group can react with an electrophilic group such as tosylate, mesylate, halo(chloro, bromo, fluoro, iodo), N-hydroxysuccinimidyl ester (NHS), and the like. Thiols can react with maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)).

Suitable toxin moieties and toxins include, for example, a maytansinoid (e.g., maytansinol, e.g., DM1, DM4), a taxane, a calicheamicin, a duocarmycin, or derivatives thereof. The maytansinoid can be, for example, maytansinol or a maytansinol analogue. Examples of maytansinol analogs include those having a modified aromatic ring (e.g., C-19-decloro, C-20-demethoxy, C-20-acyloxy) and those having modifications at other positions (e.g., C-9-CH, C-14-alkoxymethyl, C-14-hydroxymethyl or aceloxymethyl, C-15-hydroxy/acyloxy, C-15-methoxy, C-18-N-demethyl, 4,5-deoxy). Maytansinol and maytansinol analogs are described, for example, in U.S. Pat. Nos. 5,208,020 and 6,333,410, the contents of which are incorporated herein by reference. Maytansinol can be coupled to antibodies and antibody fragmetns using, e.g., an N-succinimidyl 3-(2-pyridyldithio)proprionate (also known as N-succinimidyl 4-(2-pyridyldithio)pentanoate (or SPP), 4-succinimidyl-oxycarbonyl-a-(2-pyridyldithio)-toluene (SMPT), N-succinimidyl-3-(2-pyridyldithio)butyrate (SDPB), 2 iminothiolane, or S-acetylsuccinic anhydride. The taxane can be, for example, a taxol, taxotere, or novel taxane (see, e.g., WO 01/38318). The calicheamicin can be, for example, a bromo-complex calicheamicin (e.g., an alpha, beta or gamma bromo-complex), an iodo-complex calicheamicin (e.g., an alpha, beta or gamma iodo-complex), or analogs and mimics thereof. Bromo-complex calicheamicins include I1-BR, I2-BR, I3-BR, I4-BR, J1-BR, J2-BR and K1-BR. Iodo-complex calicheamicins include I1-BR, I2-BR, I3-BR, I4-BR, J1-BR, J2-BR and K1-BR. Calicheamicin and mutants, analogs and mimics thereof are described, for example, in U.S. Pat. Nos. 4,970,198; 5,264,586; 5,550,246; 5,712,374, and 5,714,586, the contents of each of which are incorporated herein by reference. Duocarmycin analogs (e.g., KW-2189, DC88, DC89 CBI-TMI, and derivatives thereof are described, for example, in U.S. Pat. No. 5,070,092, U.S. Pat. No. 5,187,186, U.S. Pat. No. 5,641,780, U.S. Pat. No. 5,641,780, U.S. Pat. No. 4,923,990, and U.S. Pat. No. 5,101,038, the contents of each of which are incorporated herein by reference.

Examples of other toxins include, but are not limited to antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065 (see U.S. Pat. Nos. 5,475,092, 5,585,499, 5,846,545), melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, mitomycin, puromycin anthramycin (AMC)), duocarmycin and analogs or derivatives thereof, and anti-mitotic agents (e.g., vincristine, vinblastine, taxol, auristatins (e.g., auristatin E) and maytansinoids, and analogs or homologs thereof.

The toxin can also be a surface active toxin, such as a toxin that is a free radical generator (e.g. selenium containing toxin moieties), or radionuclide containing moiety. Suitable radionuclide containing moieties, include for example, moieties that contain radioactive iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹ mTc), phosphorus (³²P), rhodium sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

The toxin can be a protein, polypeptide or peptide, from bacterial sources, e.g., diphtheria toxin, pseudomonas exotoxin (PE) and plant proteins, e.g., the A chain of ricin (RTA), the ribosome inactivating proteins (RIPs) gelonin, pokeweed antiviral protein, saporin, and dodecandron are contemplated for use as toxins.

Antisense compounds of nucleic acids designed to bind, disable, promote degradation or prevent the production of the mRNA responsible for generating a particular target protein can also be used as a toxin. Antisense compounds include antisense RNA or DNA, single or double stranded, oligonucleotides, or their analogs, which can hybridize specifically to individual mRNA species and prevent transcription and/or RNA processing of the mRNA species and/or translation of the encoded polypeptide and thereby effect a reduction in the amount of the respective encoded polypeptide. Ching, et al., Proc. Natl. Acad. Sci. U.S.A. 86: 10006-10010 (1989); Broder, et al., Ann. Int. Med. 113: 604-618 (1990); Loreau, et al., FEBS Letters 274: 53-56 (1990); Useful antisense therapeutics include for example: Veglin™ (VasGene) and OGX-011 (Oncogenix).

Toxins can also be photoactive agents. Suitable photoactive agents include porphyrin-based materials such as porfimer sodium, the green porphyrins, chlorin E6, hematoporphyrin derivative itself, phthalocyanines, etiopurpurins, texaphrin, and the like.

The toxin can be an antibody or antibody fragment that binds an intracellular target, such as a dAb that binds an intracellular target (an intrabody). Such antibodies or antibody fragments (dAbs) can be directed to defined subcellular compartments or targets. For example, the antibodies or antibody fragments (dAbs) can bind an intracellular target selected from erbB2, EGFR, BCR-ABL, p21 Ras, Caspase3, Caspase7, Bcl-2, p53, Cyclin E, ATF-1/CREB, HPV16 E7, HP1, Type IV collagenases, cathepsin L as well as others described in Kontermann, R. E., Methods, 34:163-170 (2004), incorporated herein by reference in its entirety.

Polypeptide Domains that Bind IL-4

The invention provides ligands comprising polypeptide domains (e.g., immunoglobulin single variable domains, dAb monomers) that have a binding site with binding specificity for IL-13 and domains with a binding site with binding specificity for IL-4. In embodiments, the polypeptide domain (e.g., dAb) binds to IL-4 with an affinity (KD; KD=K_{off}(kd)/Kₒₙ (ka)) of 300 nM to 1 pM (i.e., 3×10⁻⁷ to 5×10⁻¹²M), in one embodiment 50 nM to 1 pM, 5 nM to 1 pM or 1 nM to 1 pM, for example a K_{D} of 1×10⁻⁷M or less, eg, 1×10⁻⁸ M or less, 1×10⁻⁹ M or less, 1×10⁴⁰ M or less or 1×10⁻¹¹M or less; and/or a K_{off} rate constant of 5×10⁻¹ s⁻¹ to 1×10⁻⁷s⁻¹, eg, 1×10⁻² s¹ to 1×10⁻⁶ s¹, 5×10⁻³ s⁻¹ to 1×10⁻⁵ s⁻¹, 5×10⁻¹ s⁻¹ or less, 1×10⁻² s¹ or less, 1×10⁻³ s⁻¹ or less, 1×10⁻⁴ s⁻¹ or less, 1×10⁻⁵ s⁻¹ or less, or 1×10⁻⁶s⁻¹ or less as determined by surface plasmon resonance.

In some embodiments, the polypeptide domain that has a binding site with binding specificity for IL-4 competes for binding to IL-4 with a dAb selected from the group consisting of any DOM9 dAb disclosed in WO2007/085815A2, the amino acid and nucleotide sequences for which are expressly incorporated herein by refrence for possible use with the present invention and for inclusion in claims herein.

In some embodiments, the polypeptide domain that has a binding site with binding specificity for IL-4 competes for binding to IL-4 with a dAb selected from the group consisting of DOM9-155-77 (SEQ ID NO:2426), DOM9-155-78 (SEQ ID NO:2427), DOM9-1,2-204 (SEQ ID NO:2428), DOM9-1,2-205 (SEQ ID NO:2429), DOM9-1,2-206 (SEQ ID NO:2430), DOM9-1,2-207 (SEQ ID NO:2431), DOM9-1,2-208 (SEQ ID NO:2432), DOM9-1,2-209 (SEQ ID NO:2433), DOM9-1,2-210 (SEQ ID NO:2434), DOM9-1,2-211 (SEQ ID NO:2435), DOM9-1,2-212 (SEQ ID NO:2436), DOM9-112-213 (SEQ ID NO:2437), DOM9-1,2-214 (SEQ ID NO:2438), DOM9-1,2-215 (SEQ ID NO:2439), DOM9-1,2-216 (SEQ ID NO:2440), DOM9-1,2-217 (SEQ ID NO:2441), DOM9-1,2-218 (SEQ ID NO:2442), DOM9-1,2-219 (SEQ ID NO:2443), DOM9-1,2-220 (SEQ ID NO:2444), DOM9-1,2-221 (SEQ ID NO:2445), DOM9-1,2-222 (SEQ ID NO:2446), DOM9-112-223 (SEQ ID NO:2447), DOM9-1,2-224 (SEQ ID NO:2448), DOM9-1,2-225 (SEQ ID NO:2449), DOM9-1,2-226 (SEQ ID NO:2450), DOM9-1,2-227 (SEQ ID NO:2451), DOM9-1,2-228 (SEQ ID NO:2452), DOM9-1,2-229 (SEQ ID NO:2453), DOM9-1,2-230 (SEQ ID NO:2454), DOM9-1,2-231 (SEQ ID NO:2455), DOM9-1,2-233 (SEQ ID NO:1734), DOM9-1,2-232 (SEQ ID NO:1733) and DOM9-1,2-234 (SEQ ID NO:1735) disclosed in WO2007/085815A2.

In some embodiments, the polypeptide domain that has a binding site with binding specificity for IL-4 (e.g. a dAb) comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence or a dAb selected from the group consisting of a DOM9 dAb disclosed in WO2007/85815A2.

In some embodiments, the polypeptide domain that has a binding site with binding specificity for IL-4 (e.g. a dAb) comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence or a dAb selected from the group consisting of DOM9-155-77 (SEQ ID NO:2426), DOM9-155-78 (SEQ ID NO:2427), DOM9-1,2-204 (SEQ ID NO:2428), DOM9-1,2-205 (SEQ ID NO:2429), DOM9-1,2-206 (SEQ ID NO:2430), DOM9-1,2-207 (SEQ ID NO:2431), DOM9-1,2-208 (SEQ ID NO:2432), DOM9-1,2-209 (SEQ ID NO:2433), DOM9-1,2-210 (SEQ ID NO:2434), DOM9-1,2-211 (SEQ ID NO:2435), DOM9-1,2-212 (SEQ ID NO:2436), DOM9-1,2-213 (SEQ ID NO:2437), DOM9-1,2-214 (SEQ ID NO:2438), DOM9-1,2-215 (SEQ ID NO:2439), DOM9-1,2-216 (SEQ ID NO:2440), DOM9-1,2-217 (SEQ ID NO:2441), DOM9-1,2-218 (SEQ ID NO:2442), DOM9-1,2-219 (SEQ ID NO:2443), DOM9-1,2-220 (SEQ ID NO:2444), DOM9-1,2-221 (SEQ ID NO:2445), DOM9-1,2-222 (SEQ ID NO:2446), DOM9-1,2-223 (SEQ ID NO:2447), DOM9-1,2-224 (SEQ ID NO:2448), DOM9-1,2-225 (SEQ ID NO:2449), DOM9-1,2-226 (SEQ ID NO:2450), DOM9-1,2-227 (SEQ ID NO:2451), DOM9-1,2-228 (SEQ ID NO:2452), DOM9-1,2-229 (SEQ ID NO:2453), DOM9-1,2-230 (SEQ ID NO:2454), DOM9-1,2-231 (SEQ ID NO:2455), DOM9-1,2-233 (SEQ ID NO:1734), DOM9-1,2-232 (SEQ ID NO:1735) and DOM9-1,2-234 (SEQ ID NO:1736) disclosed in W02007/085815A2.

In possible embodiments, the polypeptide domain that has a binding site with binding specificity for IL-4 comprises an amino acid sequence that has at least about 90%, at least about 91%, at least about 92%, at least about 93%, at least about 94%, at least about 95%, at least about 96%, at least about 97%, at least about 98%, or at least about 99% amino acid sequence identity with the amino acid sequence or a dAb selected from the group consisting of DOM9412-155 (SEQ ID NO:292), DOM9-1,2-168 (SEQ ID NO:305), DOM9-1,2-174 (SEQ ID NO:311), DOM9-1,2-199 (SEQ ID NO:336), DOM9-1,2-200 (SEQ ID NO:337), DOM9-44-502 (SEQ ID NO:512), DOM9-155-5 (SEQ ID NO:605), DOM9-155-25 (SEQ ID NO:617), DOM9-155-77 (SEQ ID NO:2426), DOM9-155-78 (SEQ ID NO:2427), DOM9-1,2-202 (SEQ ID NO:339), DOM9-1,2-209 (SEQ ID NO:2433), DOM9-1,2-210 (SEQ ID NO:2434) and DOM9-44-502 (SEQ ID NO:512) disclosed in W02007/085815A2. For example, the polypeptide domain that has a binding site with binding specificity for IL-4 can comprise DOM9-1,2-155 (SEQ ID NO:292), DOM9-1,2-168 (SEQ ID NO:305), DOM9-1,2-174 (SEQ ID NO:311), DOM9-1,2-199 (SEQ ID NO:336), DOM9-1,2-200 (SEQ ID NO:337), DOM9-44-502 (SEQ ID NO:512), DOM9-155-5 (SEQ ID NO:605, DOM9-155-25 (SEQ ID NO:617), DOM9-155-77 (SEQ ID NO:2426), DOM9-155-78 (SEQ ID NO:2427), DOM9-1,2-202 (SEQ ID NO:339), DOM9-1,2-209 (SEQ ID NO:2433), DOM9-1,2-210 (SEQ ID NO:2434) and DOM9-44-502 (SEQ ID NO:512) disclosed in WO2007/085815A2.

In some embodiments, the polypeptide domain that has a binding site with binding specificity for IL-4 competes with any of the dAbs disclosed herein for binding to IL-4.

In one embodiment the polypeptide domain that has a binding site with binding specificity for IL-4 is an immunoglobulin single variable domain. The polypeptide domain that has a binding site with binding specificity for IL-4 can comprise any suitable immunoglobulin variable domain, and in one embodiment comprises a human variable domain or a variable domain that comprises human framework regions. In certain embodiments, the polypeptide domain that has a binding site with binding specificity for IL-4 comprises a universal framework, as described herein.

The universal framework can be a V_{L} framework (Vλ or Vκ), such as a framework that comprises the framework amino acid sequences encoded by the human germline DPK1, DPK2, DPK3, DPK4, DPK5, DPK6, DPK7, DPK8, DPK9, DPK10, DPK12, DPK13, DPK15, DPK16, DPK18, DPK19, DPK20, DPK21, DPK22, DPK23, DPK24, DPK25, DPK26 or DPK28 immunoglobulin gene segment. If desired, the V_{L} framework can further comprise the framework amino acid sequence encoded by the human germline J_{κ}I, J_{κ}2, J_{κ}3, J_{κ}4, or J_{κ}5 immunoglobulin gene segment.

In other embodiments the universal framework can be a V_{H} framework, such as a framework that comprises the framework amino acid sequences encoded by the human germline DP4, DP7, DP8, DP9, DP10, DP31, DP33, DP38, DP45, DP46, DP47, DP49, DP50, DP51, DP53, DP54, DP65, DP66, DP67, DP68 or DP69 immunoglobulin gene segment. If desired, the V_{H} framework can further comprise the framework amino acid sequence encoded by the human germline J_{H}1, J_{H}2, J_{H}3, J_{H}4, J_{H}4b, J_{H}5 and J_{H}6 immunoglobulin gene segment.

In certain embodiments, the polypeptide domain that has a binding site with binding specificity for IL-4 comprises one or more framework regions comprising an amino acid sequence that is the same as the amino acid sequence of a corresponding framework region encoded by a human germline antibody gene segment, or the amino acid sequences of one or more of said framework regions collectively comprise up to 5 amino acid differences relative to the amino acid sequence of said corresponding framework region encoded by a human germline antibody gene segment.

In other embodiments, the amino acid sequences of FW1, FW2, FW3 and FW4 of the polypeptide domain that have a binding site with binding specificity for IL-4 are the same as the amino acid sequences of corresponding framework regions encoded by a human germline antibody gene segment, or the amino acid sequences of FW1, FW2, FW3 and FW4 collectively contain up to 10 amino acid differences relative to the amino acid sequences of corresponding framework regions encoded by said human germline antibody gene segment.

In other embodiments, the polypeptide domain that has a binding site with binding specificity for IL-4 comprises FW1, FW2 and FW3 regions, and the amino acid sequence of said FW1, FW2 and FW3 regions are the same as the amino acid sequences of corresponding framework regions encoded by human germline antibody gene segments.

In particular embodiments, the polypeptide domain that has a binding site with binding specificity for IL-4 comprises the DPK9 V_{L} framework, or a V_{H} framework selected from the group consisting of DP47, DP45 and DP38. The polypeptide domain that has a binding site with binding specificity for IL-4 can comprise a binding site for a generic ligand, such as protein A, protein L and protein G.

The ligand of the invention (e.g., ligand that has binding specificity for IL-4 and IL-13, ligand that has binding specificity for IL-4) can comprise a non-immunoglobulin binding moiety that has binding specificity for IL-4 and in one embodiment inhibits a function of IL-4 (e.g., binding to receptor), wherein the non-immunoglobulin binding moiety comprises one, two or three of the CDRs of a V_{H}, V_{L} or V_{HH} that binds IL-4 and a suitable scaffold. In certain embodiments, the non-immunoglobulin binding moiety comprises CDR3 but not CDR1 or CDR2 of a V_{H}, V_{L} or V_{HH} that binds IL-4 and a suitable scaffold. In other embodiments, the non-immunoglobulin binding moiety comprises CDR1 and CDR2, but not CDR3 of a V_{H}, V_{L} or V_{HH} that binds IL-4 and a suitable scaffold. In other embodiments, the non-immunoglobulin binding moiety comprises CDR1, CDR2 and CDR3 of a V_{H}, V_{L} or V_{HH} that binds IL-4 and a suitable scaffold. In one embodiment, the CDR or CDRs of the ligand of these embodiments is a CDR or CDRs of an anti-IL-4 dAb described herein. In one embodiment, the non-immunoglobulin binding moiety comprises one, two, or three of the CDRs of one of the anti-IL-4 dAbs disclosed herein. In other embodiments, the ligand (e.g., ligand that has binding specificity for IL-4 and IL-13, ligand that has binding specificity for IL-4) comprises only CDR3 of a V_{H}, V_{L} or V_{HH} that binds IL-4. The non-immunoglobulin domain can comprise an amino acid sequence that has one or more regions that have sequence identity to one, two or three of the CDRs of an anti-IL-4 dAb described herein. For example, the non-immunoglobulin domain can have an amino acid sequence that contains at least about 50%, at least about 60%, at least about 70%, at least about 80%, or at least about 90% sequence identity with CDR1, CDR2 and/or CDR3 of an anti-IL-4 dAb disclosed herein. In one embodiment, the non-immunoglobulin binding moiety comprises one, two, or three of the CDRs of DOM9-44-502 (SEQ ID NO:512), DOM9-155-5 (SEQ ID NO:605), DOM9-155-25 (SEQ ID NO:617), DOM9-1,2-155 (SEQ ID NO:292), DOM9-1,2-168 (SEQ ID NO:305), DOM9-1,2-174 (SEQ ID NO:311), DOM9-1,2-199 (SEQ ID NO:336), and DOM9-1,2-200 (SEQ ID NO:337) disclosed in WO2007/085815A2.

In certain embodiments, the polypeptide domain that has a binding site with binding specificity for IL-4 is substantially resistant to aggregation. For example, in some embodiments, less than about 10%, less than about 9%, less than about 8%, less than about 7%, less than about 6%, less than about 5%, less than about 4%, less than about 3%, less than about 2% or less than about 1% of the polypeptide domain that has a binding site with binding specificity for IL-4 aggregates when a 1-5 mg/ml, 5-10 mg/ml, 10-20 mg/ml, 20-50 mg/ml, 50-100 mg/ml, 100-200 mg/ml or 200-500 mg/ml solution of ligand or dAb in a solvent that is routinely used for drug formulation such as saline, buffered saline, citrate buffer saline, water, an emulsion, and, any of these solvents with an acceptable excipient such as those approved by the FDA, is maintained at about 22° C., 22-25° C., 25-30° C., 30-37° C., 37-40° C., 40-50° C., 50-60° C., 60-70° C., 70-80° C., 15-20° C., 10-15° C., 5-10° C., 2-5° C., 0-2° C., −10° C. to 0° C., −20° C. to −10° C., −40° C. to −20° C., −60° C. to −40° C., or −80° C. to −60° C., for a period of about time, for example, 10 minutes, 1 hour, 8 hours, 24 hours, 2 days, 3 days, 4 days, 1 week, 2 weeks, 3 weeks, 1 month, 2 months, 3 months, 4 months, 6 months, 1 year, or 2 years.

Aggregation can be assessed using any suitable method, such as, by microscopy, assessing turbidity of a solution by visual inspection or spectroscopy or any other suitable method. In one embodiment, aggregation is assessed by dynamic light scattering. Polypeptide domains that have a binding site with binding specificity for IL-4 that are resistant to aggregation provide several advantages. For example, such polypeptide domains that have a binding site with binding specificity for IL-4 can readily be produced in high yield as soluble proteins by expression using a suitable biological production system, such as E. coli, and can be formulated and/or stored at higher concentrations than conventional polypeptides, and with less aggregation and loss of activity.

In addition, the polypeptide domain that has a binding site with binding specificity for IL-4 that is resistant to aggregation can be produced more economically than other antigen- or epitope-binding polypeptides (e.g., conventional antibodies). For example, generally, preparation of antigen- or epitope-binding polypeptides intended for in vivo applications includes processes (e.g., gel filtration) that remove aggregated polypeptides. Failure to remove such aggregates can result in a preparation that is not suitable for in vivo applications because, for example, aggregates of an antigen-binding polypeptide that is intended to act as an antagonist can function as an agonist by inducing cross-linking or clustering of the target antigen. Protein aggregates can also reduce the efficacy of therapeutic polypeptide by inducing an immune response in the subject to which they are administered.

In contrast, the aggregation resistant polypeptide domain that has a binding site with binding specificity for IL-4 of the invention can be prepared for in vivo applications without the need to include process steps that remove aggregates, and can be used in in vivo applications without the aforementioned disadvantages caused by polypeptide aggregates.

In some embodiments, a polypeptide domain that has a binding site with binding specificity for IL-4 unfolds reversibly when heated to a temperature (Ts) and cooled to a temperature (Tc), wherein Ts is greater than the melting temperature (Tm) of the polypeptide domain that has a binding site with binding specificity for IL-4, and Tc is lower than the melting temperature of the polypeptide domain that has a binding site with binding specificity for IL-4. For example, a polypeptide domain that has a binding site with binding specificity for IL-4 can unfold reversibly when heated to 80° C. and cooled to about room temperature. A polypeptide that unfolds reversibly loses function when unfolded but regains function upon refolding. Such polypeptides are distinguished from polypeptides that aggregate when unfolded or that improperly refold (misfolded polypeptides), i.e., do not regain function.

Polypeptide unfolding and refolding can be assessed, for example, by directly or indirectly detecting polypeptide structure using any suitable method. For example, polypeptide structure can be detected by circular dichroism (CD) (e.g., far-UV CD, near-UV CD), fluorescence (e.g., fluorescence of tryptophan side chains), susceptibility to proteolysis, nuclear magnetic resonance (NMR), or by detecting or measuring a polypeptide function that is dependent upon proper folding (e.g., binding to target ligand, binding to generic ligand). In one example, polypeptide unfolding is assessed using a functional assay in which loss of binding function (e.g., binding a generic and/or target ligand, binding a substrate) indicates that the polypeptide is unfolded.

The extent of unfolding and refolding of a polypeptide domain that has a binding site with binding specificity for IL-4 can be determined using an unfolding or denaturation curve. An unfolding curve can be produced by plotting temperature as the ordinate and the relative concentration of folded polypeptide as the abscissa. The relative concentration of folded polypeptide domain that has a binding site with binding specificity for IL-4 can be determined directly or indirectly using any suitable method (e.g., CD, fluorescence, binding assay). For example, a polypeptide domain that has a binding site with binding specificity for IL-4 solution can be prepared and ellipticity of the solution determined by CD. The ellipticity value obtained represents a relative concentration of folded ligand or dAb monomer of 100%. The polypeptide domain that has a binding site with binding specificity for IL-4 in the solution is then unfolded by incrementally raising the temperature of the solution and ellipticity is determined at suitable increments (e.g., after each increase of one degree in temperature). The polypeptide domain that has a binding site with binding specificity for IL-4 in solution is then refolded by incrementally reducing the temperature of the solution and ellipticity is determined at suitable increments. The data can be plotted to produce an unfolding curve and a refolding curve. The unfolding and refolding curves have a characteristic sigmoidal shape that includes a portion in which the polypeptide domain that has a binding site with binding specificity for IL-4 molecules is folded, an unfolding/refolding transition in which the polypeptide domain that has a binding site with binding specificity for IL-4 molecules is unfolded to various degrees, and a portion in which polypeptide domain that has a binding site with binding specificity for IL-4 is unfolded. The y-axis intercept of the refolding curve is the relative amount of refolded polypeptide domain that has a binding site with binding specificity for IL-4 recovered. A recovery of at least about 50%, or at least about 60%, or at least about 70%, or at least about 75%, or at least about 80%, or at least about 85%, or at least about 90%, or at least about 95% is indicative that the ligand or dAb monomer unfolds reversibly.

In a possible embodiment, reversibility of unfolding of polypeptide domain that has a binding site with binding specificity for IL-4 is determined by preparing a polypeptide domain that has a binding site with binding specificity for IL-4 solution and plotting heat unfolding and refolding curves. The polypeptide domain that has a binding site with binding specificity for IL-4 solution can be prepared in any suitable solvent, such as an aqueous buffer that has a pH suitable to allow polypeptide domain that has a binding site with binding specificity for IL-4 to dissolve (e.g., pH that is about 3 units above or below the isoelectric point (pI)). The polypeptide domain that has a binding site with binding specificity for IL-4 solution is concentrated enough to allow unfolding/folding to be detected. For example, the ligand or dAb monomer solution can be about 0.1 μM to about 100 μM, or about 1 μM to about 10 μM.

If the melting temperature (Tm) of the polypeptide domain that has a binding site with binding specificity for IL-4 is known, the solution can be heated to about ten degrees below the Tm (Tm-10) and folding assessed by ellipticity or fluorescence (e.g., far-UV CD scan from 200 nm to 250 nm, fixed wavelength CD at 235 nm or 225 nm; tryptophan fluorescent emission spectra at 300 to 450 nm with excitation at 298 nm) to provide 100% relative folded ligand or dAb monomer. The solution is then heated to at least ten degrees above Tm (Tm+10) in predetermined increments (e.g., increases of about 0.1 to about 1 degree), and ellipticity or fluorescence is determined at each increment. Then, the polypeptide domain that has a binding site with binding specificity for IL-4 is refolded by cooling to at least Tm-10 in predetermined increments and ellipticity or fluorescence determined at each increment. If the melting temperature of the polypeptide domain that has a binding site with binding specificity for IL-4 is not known, the solution can be unfolded by incrementally heating from about 25° C. to about 100° C. and then refolded by incrementally cooling to at least about 25° C., and ellipticity or fluorescence at each heating and cooling increment is determined. The data obtained can be plotted to produce an unfolding curve and a refolding curve, in which the y-axis intercept of the refolding curve is the relative amount of refolded protein recovered. In some embodiments, the polypeptide domain that has a binding site with binding specificity for VEGF does not comprise a Camelid immunoglobulin variable domain, or one or more framework amino acids that are unique to immunoglobulin variable domains encoded by Camelid germ line antibody gene segments.

In one embodiment, the polypeptide domain that has a binding site with binding specificity for IL-4 is secreted in a quantity of at least about 0.5 mg/L when expressed in E. coli or in Pichia species (e.g., P. pastoris). In other possible embodiments, polypeptide domain that has a binding site with binding specificity for IL-4 is secreted in a quantity of at least about 0.75 mg/L, at least about 1 mg/L, at least about 4 mg/L, at least about 5 mg/L, at least about 10 mg/L, at least about 15 mg/L, at least about 20 mg/L, at least about 25 mg/L, at least about 30 mg/L, at least about 35 mg/L, at least about 40 mg/L, at least about 45 mg/L, or at least about 50 mg/L, or at least about 100 mg/L, or at least about 200 mg/L, or at least about 300 mg/L, or at least about 400 mg/L, or at least about 500 mg/L, or at least about 600 mg/L, or at least about 700 mg/L, or at least about 800 mg/L, at least about 900 mg/L, or at least about 1 g/L when expressed in E. coli or in Pichia species (e.g., P. P pastoris). In other possible embodiments, a polypeptide domain that has a binding site with binding specificity for IL-4 is secreted in a quantity of at least about 1 mg/L to at least about 1 g/L, at least about 1 mg/L to at least about 750 mg/L, at least about 100 mg/L to at least about 1 g/L, at least about 200 mg/L to at least about 1 g/L, at least about 300 mg/L to at least about 1 g/L, at least about 400 mg/L to at least about 1 g/L, at least about 500 mg/L to at least about 1 g/L, at least about 600 mg/L to at least about 1 g/L, at least about 700 mg/L to at least about 1 g/L, at least about 800 mg/L to at least about 1 g/L, or at least about 900 mg/L to at least about 1 g/L when expressed in E. coli or in Pichia species (e.g., P. pastoris). Although, polypeptide domain that has a binding site with binding specificity for IL-4 described herein can be secretable when expressed in E. coli or in Pichia species (e.g., P. pastoris), they can be produced using any suitable method, such as synthetic chemical methods or biological production methods that do not employ E. coli or Pichia species.

Polypeptide Domains that Bind IL-13

The invention provides polypeptide domains (e.g., dAb) that have a binding site with binding specificity for IL-13. In possible embodiments, the polypeptide domain (e.g., dAb) binds to IL-13 with an affinity (KD; KID=K_{off}(kd)/Kₒₙ (ka)) of 300 nM to 1 pM (i.e., 3×10⁻⁷ to 5×10⁻¹²M), eg, 100 nM to 1 pM, or 50 nM to 10 pM, 10 nM to 100 pM or 1 nM, for example a K_{D} of 1×10⁻⁷ M or less, 1×10⁻⁸M or less, about 1×10⁻⁹ M or less, 1×10⁻¹⁰ M or less or 1×10⁻¹¹M or less; and/or a K_{off} rate constant of 5×10⁻¹ s⁻¹ to 1×10⁻⁷ s⁻¹ eg, 1×10⁻² s⁻¹ to 1×10⁻⁶ s⁻¹, 5×10³ s⁻¹ to 1×10⁻⁵ s⁻¹, for example 5×10⁻¹ s⁻¹ or less, 1×10⁻² s⁻¹ or less, 1×10⁻³ s⁻¹ or less, 1×10⁻⁴ s⁻¹ or less, 1×10⁻⁵ s⁻¹ or less, or 1×10⁻⁶s⁻¹ or less as determined by surface plasmon resonance.

In some embodiments, a polypeptide domain that has a binding site with binding specificity for IL-13 competes for binding to IL-13 with a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10), and optionally DOM10-53-474 (SEQ ID NO:1). For example, the binding of the polypeptide domain that has a binding site with binding specificity for IL-13 to IL-13 is inhibited by a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10), and optionally DOM10-53-474 (SEQ ID NO:1). In other examples, the polypeptide domain that has a binding site with binding specificity for IL-13 has the epitopic specificity of a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10), and optionally DOM10-53-474 (SEQ ID NO:1).

In some embodiments, the polypeptide domain that has a binding site with binding specificity for IL-13 (e.g., a dAb) comprises an amino acid sequence that has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence or a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10), and optionally DOM10-53-474 (SEQ ID NO:1).

In some embodiments, the polypeptide domain that has a binding site with binding specificity for IL-13 competes with any of the dAbs disclosed herein for binding to IL-13.

In one embodiment the polypeptide domain that has a binding site with binding specificity for IL-13 is an immunoglobulin single variable domain. The polypeptide domain that has a binding site with binding specificity for IL-13 can comprise any suitable immunoglobulin variable domain, and in one embodiment comprises a human variable domain or a variable domain that comprises human framework regions. In certain embodiments, the polypeptide domain that has a binding site with binding specificity for IL-13 comprises a universal framework, as described herein.

The ligand of the invention (e.g., ligand that has binding specificity for IL-4 and IL-13, ligand that has binding specificity for IL-13) can comprise a non-immunoglobulin binding moiety that has binding specificity for IL-13 and inhibits a function of IL-13 (e.g., binding to receptor), wherein the non-immunoglobulin binding moiety comprises one, two or three of the CDRs of a V_{H}, V_{L} or V_{HH} that binds IL-13 and a suitable scaffold. In certain embodiments, the non-immunoglobulin binding moiety comprises CDR3 but not CDR1 or CDR2 of a V_{H}, V_{L} or V_{HH} that binds IL-13 and a suitable scaffold. In other embodiments, the non-immunoglobulin binding moiety comprises CDR1 and CDR2, but not CDR3 of a V_{H}, V_{L} or V_{HH} that binds IL-13 and a suitable scaffold. In other embodiments, the non-immunoglobulin binding moiety comprises CDR1, CDR2 and CDR3 of a V_{H}, V_{L} or V_{HH} that binds IL-13 and a suitable scaffold. In one embodiment, the CDR or CDRs of the ligand of these embodiments is a CDR or CDRs of an anti-IL-13 dAb described herein. In one embodiment, the non-immunoglobulin binding moiety comprises one, two, or three of the CDRs of one of the anti-IL-13 dAbs disclosed herein. In other embodiments, the ligand (e.g., ligand that has binding specificity for IL-4 and IL-13, ligand that has binding specificity for IL-13) comprises only CDR3 of a V_{H}, V_{L} or V_{HH} that binds IL-13. The non-immunoglobulin domain can comprise an amino acid sequence that has one or more regions that have sequence identity to one, two or three of the CDRs of an anti-IL-13 dAb described herein. For example, the non-immunoglobulin domain can have an amino acid sequence that contains at least about 50%, at least about 60%, at least about 70%, at least about 80%, or at least about 90% sequence identity with CDR1, CDR2 and/or CDR3 of an anti-IL13 dAb disclosed herein. In certain possible embodiments, the non-immunoglobulin binding moiety comprises one, two, or three of the CDRs of DOM10-275-78 (SEQ ID NO:6), DOM10-275-94 (SEQ ID NO:7), DOM10-275-99 (SEQ ID NO:8), DOM10-275-100 (SEQ ID NO:9) or DOM10-275-101 (SEQ ID NO:10), and optionally DOM10-53-474 (SEQ ID NO:1).

In certain embodiments, a polypeptide domain that has a binding site with binding specificity for IL-13 resists aggregation, unfolds reversibly, comprises a framework region and/or is secreted as described above for the polypeptide domain that has a binding site with binding specificity for IL-4

dAb Monomers that Bind Serum Albumin

The ligands of the invention can further comprise a dAb monomer that binds serum albumin (SA) with a K_{d} of 1 nM to 500 μM (i.e., ×10⁻⁹ to 5×10⁻⁴), in one embodiment 100 nM to 10 μM. In one embodiment, for a ligand comprising an anti-SA dAb, the binding (e.g. K_{d} and/or K_{off} as measured by surface plasmon resonance, (e.g., using BiaCore)) of the ligand its target(s) is from 1 to 100000 times (in one embodiment 100 to 100000, 1000 to 100000, or 10000 to 100000 times) stronger than for SA. In one embodiment, the serum albumin is human serum albumin (HSA). In one embodiment, the first dAb (or a dAb monomer) binds SA (e.g., HSA) with a K_{d} of approximately 50, 70, 100, 150 or 200 nM.

In certain embodiments, the dAb monomer that binds SA resists aggregation, unfolds reversibly and/or comprises a framework region as described above for dAb monomers that bind IL-4.

In particular embodiments, the antigen-binding fragment of an antibody that binds serum albumin is a dAb that binds human serum albumin. In certain embodiments, the dAb binds human serum albumin and competes for binding to albumin with a dAb selected from the group consisting of MSA-16, MSA-26 (See WO04003019 for disclosure of these sequences, which sequences and their nucleic acid counterpart are incorporated herein by reference and form part of the disclosure of the present text),

DOM7m-16 (SEQ ID NO: 473), DOM7m-12 (SEQ ID NO: 474), DOM7m-26 (SEQ ID NO: 475), DOM7r-1 (SEQ ID NO: 476), DOM7r-3 (SEQ ID NO: 477), DOM7r-4 (SEQ ID NO: 478), DOM7r-5 (SEQ ID NO: 479), DOM7r-7 (SEQ ID NO: 480), DOM7r-8 (SEQ ID NO: 481), DOM7h-2 (SEQ ID NO: 482), DOM7h-3 (SEQ ID NO: 483), DOM7h-4 (SEQ ID NO: 484), DOM7h-6 (SEQ ID NO: 485), DOM7h-1 (SEQ ID NO: 486), DOM7h-7 (SEQ ID NO: 487), DOM7h-22 (SEQ ID NO: 489), DOM7h-23 (SEQ ID NO: 490), DOM7h-24 (SEQ ID NO: 491), DOM7h-25 (SEQ ID NO: 492), DOM7h-26 (SEQ ID NO: 493), DOM7h-21 (SEQ ID NO: 494), DOM7h-27 (SEQ ID NO: 495), DOM7h-8 (SEQ ID NO: 496), DOM7r-13 (SEQ ID NO: 497), DOM7r-14 (SEQ ID NO: 498), DOM7r-15 (SEQ ID NO: 499), DOM7r-16 (SEQ ID NO: 500), DOM7r-17 (SEQ ID NO: 501), DOM7r-18 (SEQ ID NO: 502), DOM7r-19 (SEQ ID NO: 503), DOM7r-20 (SEQ ID NO: 504), DOM7r-21 (SEQ ID NO: 505), DOM7r-22 (SEQ ID NO: 506), DOM7r-23 (SEQ ID NO: 507), DOM7r-24 (SEQ ID NO: 508), DOM7r-25 (SEQ ID NO: 509), DOM7r-26 (SEQ ID NO: 510), DOM7r-27 (SEQ ID NO: 511), DOM7r-28 (SEQ ID NO: 512), DOM7r-29 (SEQ ID NO: 513), DOM7r-30 (SEQ ID NO: 514), DOM7r-31 (SEQ ID NO: 515), DOM7r-32 (SEQ ID NO: 516), DOM7r-33 (SEQ ID NO: 517) (See WO2007080392 for disclosure of these sequences, which sequences and their nucleic acid counterpart are incorporated herein by reference and form part of the disclosure of the present text; the SEQ ID No's in this paragraph are those that appear in WO2007080392),

dAb8 (dAb10), dAb 10, dAb36, dAb7r20 (DOM7r20), dAb7r21 (DOM7r21), dAb7r22 (DOM7r22), dAb7r23 (DOM7r23), dAb7r24 (DOM7r24), dAb7r25 (DOM7r25), dAb7r26 (DOM7r26), dAb7r27 (DOM7r27), dAb7r28 (DOM7r28), dAb7r29 (DOM7r29), dAb7r29 (DOM7r29), dAb7r31 (DOM7r31), dAb7r32 (DOM7r32), dAb7r33 (DOM7r33), dAb7r33 (DOM7r33), dAb7h22 (DOM7h22), dAb7h23 (DOM7h23), dAb7h24 (DOM7h24), dAb7h25 (DOM7h25), dAb7h26 (DOM7h26), dAb7h27 (DOM7h27), dAb7h30 (DOM7h30), dAb7h31 (DOM7h31), dAb2 (dAbs 4,7,41), dAb4, dAb7, dAb11, dAb12 (dAb7 m12), dAb13 (dAb 15), dAb15, dAb 16 (dAb21, dAb7 m16), dAb17, dAb18, dAb19, dAb21, dAb22, dAb23, dAb24, dAb25 (dAb26, dAb7 m26), dAb27, dAb30 (dAb35), dAb31, dAb33, dAb34, dAb35, dAb38 (dAb54), dAb41, dAb46 (dAbs 47, 52 and 56), dAb47, dAb52, dAb53, dAb54, dAb55, dAb56, dAb7 m12, dAb7 m16, dAb7 m26, dAb7r1 (DOM 7r1), dAb7r3 (DOM7r3), dAb7r4 (DOM7r4), dAb7r5 (DOM7r5), dAb7r7 (DOM7r7), dAb7r8 (DOM7r8), dAb7r13 (DOM7r13), dAb7r14 (DOM7r14), dAb7r15 (DOM7r15), dAb7r16 (DOM7r16), dAb7r17 (DOM7r17), dAb7r18 (DOM7r18), dAb7r19 (DOM7r19), dAb7h1 (DOM7h1), dAb7h2 (DOM7h2), dAb7h6 (DOM7h6), dAb7h7 (DOM7h7), dAb7h8 (DOM7h8), dAb7h9 (DOM7h9), dAb7h10 (DOM7h10), dAb7h11 (DOM7h11), dAb7h12 (DOM7h12), dAb7h13 (DOM7h13), dAb7h14 (DOM7h14), dAb7p1 (DOM7p1), and dAb7p2 (DOM7p2) (see WO2008096158 for disclosure of these sequences, which sequences and their nucleic acid counterpart are incorporated herein by reference and form part of the disclosure of the present text),

DOM7h-14-10, DOM7h-14-18, DOM7h-14-28, DOM7h-14-19 and DOM7h-14-36 (see copending application U.S. Ser. No. 61/163,990 filed 27 Mar. 2009 which sequences and their nucleic acid counterpart are incorporated herein by reference and form part of the disclosure of the present text),

DOM7h-11-3, DOM7h-11-15, DOM7h-11-12, DOM7h-11-18, and DOM7h-11-19 (see copending application U.S. Ser. No. 61/163,987 filed 27 Mar. 2009 which sequences and their nucleic acid counterpart are incorporated herein by reference and form part of the disclosure of the present text).

Alternative names are shown in brackets after the dAb, e.g. dAb8 has an alternative name which is dAb10 i.e. dAb8 (dAb10). Relevant sequences are also set out in FIGS. 51a and b of W02008149148, incorporated herein by reference.

In certain embodiments, the dAb binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with the amino acid sequence of a dAb selected from the group set out above, eg DOM7h-14-10, DOM7h-11-3 or DOM7h-11-15.

Amino acid sequence identity is in one embodiment determined using a suitable sequence alignment algorithm and default parameters, such as BLAST P (Karlin and Altschul, Proc. Natl. Acad. Sci. USA, 87(6):2264-2268 (1990)).

In other embodiments, the antigen-binding fragment of an antibody that binds serum albumin is a dAb that binds human serum albumin and comprises the CDRs of any of the foregoing amino acid sequences.

Suitable Camelid V_{HH} that bind serum albumin include those disclosed in WO 2004/041862 (Ablynx N.V.) and herein, such as Sequence A (SEQ ID NO:1778), Sequence B (SEQ ID NO:1779), Sequence C (SEQ ID NO:1780), Sequence D (SEQ ID NO:1781), Sequence E (SEQ ID NO:1782), Sequence F (SEQ ID NO:1783), Sequence G (SEQ ID NO:1784), Sequence H (SEQ ID NO:1785), Sequence I (SEQ ID NO:1786), Sequence J (SEQ ID NO:1787), Sequence K (SEQ ID NO:1788), Sequence L (SEQ ID NO:1789), Sequence M (SEQ ID NO:1790), Sequence N (SEQ ID NO:1791), Sequence 0 (SEQ ID NO:1792), Sequence P (SEQ ID NO:1793), Sequence Q (SEQ ID NO:1794). In certain embodiments, the Camelid V_{HH} binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with any one of SEQ ID NOS:1778-1794.

Amino acid sequence identity is in one embodiment determined using a suitable sequence alignment algorithm and default parameters, such as BLAST P (Karlin and Altschul, Proc. Natl. Acad. Sci. USA, 87(6):2264-2268 (1990)).

In some embodiments, the ligand comprises an anti-serum albumin dAb that competes with any anti-serum albumin dAb disclosed herein for binding to serum albumin (e.g., human serum albumin).

### Nucleic Acid Molecules, Vectors and Host Cells

The invention also provides isolated and/or recombinant nucleic acid molecules encoding ligands, (dual-specific ligands and multispecific ligands) as described herein.

Nucleic acids referred to herein as “isolated” are nucleic acids which have been separated away from the nucleic acids of the genomic DNA or cellular RNA of their source of origin (e.g., as it exists in cells or in a mixture of nucleic acids such as a library), and include nucleic acids obtained by methods described herein or other suitable methods, including essentially pure nucleic acids, nucleic acids produced by chemical synthesis, by combinations of biological and chemical methods, and recombinant nucleic acids which are isolated (see e.g., Daugherty, B. L. et al., Nucleic Acids Res., 19(9): 2471-2476 (1991); Lewis, A. P. and J. S. Crowe, Gene, 101: 297-302 (1991)).

Nucleic acids referred to herein as “recombinant” are nucleic acids which have been produced by recombinant DNA methodology, including those nucleic acids that are generated by procedures which rely upon a method of artificial recombination, such as the polymerase chain reaction (PCR) and/or cloning into a vector using restriction enzymes.

In certain embodiments, the isolated and/or recombinant nucleic acid comprises a nucleotide sequence encoding a ligand, as described herein, wherein said ligand comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb that binds IL-4 disclosed herein, or a dAb that binds IL-13 disclosed herein.

For example, in some embodiments, the isolated and/or recombinant nucleic acid comprises a nucleotide sequence encoding a ligand that has binding specificity for IL-4, as described herein, wherein said ligand comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of those DOM9 dAbs referred to above.

In other embodiments, the isolated and/or recombinant nucleic acid comprises a nucleotide sequence encoding a ligand that has binding specificity for IL-13, as described herein, wherein said ligand comprises an amino acid sequence that has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10), and optionally DOM10-53-474 (SEQ ID NO: 1).

In other embodiments, the isolated and/or recombinant nucleic acid comprises a nucleotice sequence encoding a ligand that has binding specificity for IL-4, as described herein, wherein said nucleotide sequence has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% nucleotide sequence identity with a nucleotide sequence encoding an anti-IL-4 dAb selected from the group consisting of the DOM9 dAbs referred to above. In one embodiment, nucleotide sequence identity is determined over the whole length of the nucleotice sequence that encodes the selected anti-IL-4 dAb.

In other embodiments, the isolated and/or recombinant nucleic acid comprises a nucleotice sequence encoding a ligand that has binding specificity for IL-13, as described herein, wherein said nucleotide sequence has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% nucleotide sequence identity with a nucleotide sequence encoding an anti-IL-13 dAb selected from the group consisting of DOM10-275-78 (SEQ ID NO:11), DOM10-275-94 (SEQ ID NO:12), DOM10-275-99 (SEQ ID NO:13), DOM10-275-100 (SEQ ID NO:14) and DOM10-275-101 (SEQ ID NO:15), and optionally DOM10-53-474 (SEQ ID NO:2).

In some embodiments, the nucleotide sequence may be a codon-optimized version of the nucleotide sequence encoding a ligand that has binding specificity for IL-4 or IL-13, as described herein. Codon optimization of sequences is known in the art. In one embodiment, the nucleotide sequence is optimized for expression in a bacterial (e.g., E. coli or Pseudomonas sp., e.g., P. fluorescens), mammalian (e.g., CHO) or yeast host cell (e.g., Picchia or Saccharomyces, e.g., P. pastoris or S. cerevisiae).

As described above, embodiments of the invention provide codon optimized nucleotide sequences encoding polypeptides and variable domains of the invention. Codon optimized sequences of about 70% identity can be produced that encode for the same variable domain (e.g., encode for DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10)).

The invention also provides a vector comprising a recombinant nucleic acid molecule of the invention. In certain embodiments, the vector is an expression vector comprising one or more expression control elements or sequences that are operably linked to the recombinant nucleic acid of the invention The invention also provides a recombinant host cell comprising a recombinant nucleic acid molecule or vector of the invention. Suitable vectors (e.g., plasmids, phagmids), expression control elements, host cells and methods for producing recombinant host cells of the invention are well-known in the art, and examples are further described herein.

Suitable expression vectors can contain a number of components, for example, an origin of replication, a selectable marker gene, one or more expression control elements, such as a transcription control element (e.g., promoter, enhancer, terminator) and/or one or more translation signals, a signal sequence or leader sequence, and the like. Expression control elements and a signal sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for prokaryotic (e.g., lac, tac, T3, T7 promoters for E. coli) and eukaryotic (e.g., Simian Virus 40 early or late promoter, Rous sarcoma virus long terminal repeat promoter, cytomegalovirus promoter, adenovirus late promoter) hosts are available.

In addition, expression vectors typically comprise a selectable marker for selection of host cells carrying the vector, and, in the case of a replicable expression vector, an origin of replication. Genes encoding products which confer antibiotic or drug resistance are common selectable markers and may be used in prokaryotic (e.g. lactamase gene (ampicillin resistance), Tet gene for tetracycline resistance) and eukaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated. Suitable expression vectors for expression in mammalian cells and prokaryotic cells (E. coli), insect cells (Drosophila Schnieder S2 cells, Sf9) and yeast (P. methanolica, P. pastoris, S. cerevisiae) are well-known in the art.

Suitable host cells can be prokaryotic, including bacterial cells such as E. coli, B. subtilis and/or other suitable bacteria; eukaryotic cells, such as fungal or yeast cells (e.g., Pichia pastoris, Aspergillus sp., Saccharomyces cerevisiae, Schizosaccharomyces pombe, Neurospora crassa), or other lower eukaryotic cells, and cells of higher eukaryotes such as those from insects (e.g., Drosophila Schnieder S2 cells, Sf9 insect cells (WO 94/26087 (O'Connor)), mammals (e.g., COS cells, such as COS-1 (ATCC Accession No. CRL-1650) and COS-7 (ATCC Accession No. CRL-1651), CHO (e.g., ATCC Accession No. CRL-9096, CHO DG44 (Urlaub, G. and Chasin, L A., Proc. Natl. Acac. Sci. USA, 77(7):4216-4220 (1980))), 293 (ATCC Accession No. CRL-1573), HeLa (ATCC Accession No. CCL-2), CV1 (ATCC Accession No. CCL-70), WOP (Dailey, L., et al., J. Virol., 54:739-749 (1985), 3T3, 293T (Pear, W. S., et al., Proc. Natl. Acad. Sci. U.S.A., 90:8392-8396 (1993)) NSO cells, SP2/0, HuT 78 cells and the like, or plants (e.g., tobacco). (See, for example, Ausubel, F. M. et al., eds. Current Protocols in Molecular Biology, Greene Publishing Associates and John Wiley & Sons Inc. (1993).) In some embodiments, the host cell is an isolated host cell and is not part of a multicellular organism (e.g., plant or animal). In possible embodiments, the host cell is a non-human host cell.

The invention also provides a method for producing a ligand (e.g., dual-specific ligand, multispecific ligand) of the invention, comprising maintaining a recombinant host cell comprising a recombinant nucleic acid of the invention under conditions suitable for expression of the recombinant nucleic acid, whereby the recombinant nucleic acid is expressed and a ligand is produced. In some embodiments, the method further comprises isolating the ligand.

The following sections of W02007/085815A2, are referred to for use with the present invention and these disclosures are incorporated herein by reference as though written herein verbatim and to provide disclosure for inclusion in claims herein:

### Preparation of Immunoglobulin Based Ligands

Library vector systems

### Library Construction

### Characterisation of Ligands

### Scaffolds for Use in Constructing Ligands

### Selection of the Main-chain Conformation

### Diversification of the Canonical Sequence

### Diversification of the Canonical Sequence as it Applies to Antibody Domains

### Combining Single Variable Domains

Domains useful in the invention, once selected, may be combined by a variety of methods known in the art, including covalent and non-covalent methods. Possible methods include the use of polypeptide linkers, as described, for example, in connection with scFv molecules (Bird et al., (1988) Science 242:423-426). Discussion of suitable linkers is provided in Bird et al. Science 242, 423-426; Hudson et al, Journal Immunol Methods 231 (1999) 177-189; Hudson et al, Proc. Nat. Acad. Sci. 85, 5879-5883. Linkers are in one embodiment flexible, allowing the two single domains to interact. One linker example is a (Gly₄ Ser)ₙ linker, where n=1 to 8, e.g., 2, 3, 4, 5 or 7. The linkers used in diabodies, which are less flexible, may also be employed (Holliger et al., (1993) Proc. Nat. Acad. Sci. U.S.A. 90:6444-6448). In one embodiment, the linker employed is not an immunoglobulin hinge region.

Variable domains may be combined using methods other than linkers. For example, the use of disulphide bridges, provided through naturally-occurring or engineered cysteine residues, may be exploited to stabilize V_{H}-V_{H}, V_{L}-V_{L} or V_{H}-V_{L} dimers (Reiter et al., (1994) Protein Eng. 7:697-704) or by remodelling the interface between the variable domains to improve the “fit” and thus the stability of interaction (Ridgeway et al., (1996) Protein Eng. 7:617-621; Zhu et al., (1997) Protein Science 6:781-788). Other techniques for joining or stabilizing variable domains of immunoglobulins, and in particular antibody V_{H} domains, may be employed as appropriate.

### Structure of Ligands

In the case that each variable domain is selected from V-gene repertoires selected for instance using phage display technology as herein described, then these variable domains comprise a universal framework region, such that is they may be recognized by a generic ligand as herein defined. The use of universal frameworks, generic ligands and the like is described in WO99/20749.

Where V-gene repertoires are used, variation in polypeptide sequence is in one embodiment located within the structural loops of the variable domains. The polypeptide sequences of either variable domain may be altered by DNA shuffling or by mutation in order to enhance the interaction of each variable domain with its complementary pair. DNA shuffling is known in the art and taught, for example, by Stemmer, 1994, Nature 370: 389-391 and U.S. Pat. No. 6,297,053, both of which are incorporated herein by reference. Other methods of mutagenesis are well known to those of skill in the art.

In general, nucleic acid molecules and vector constructs required for selection, preparation and formatting dual-specific ligands may be constructed and manipulated as set forth in standard laboratory manuals, such as Sambrook et al. (1989) Molecular Cloning: A Laboratory Manual, Cold Spring Harbor, USA.

The manipulation of nucleic acids useful in the present invention is typically carried out in recombinant vectors. As used herein, vector refers to a discrete element that is used to introduce heterologous DNA into cells for the expression and/or replication thereof. Methods by which to select or construct and, subsequently, use such vectors are well known to one of ordinary skill in the art. Numerous vectors are publicly available, including bacterial plasmids, bacteriophage, artificial chromosomes and episomal vectors. Such vectors may be used for simple cloning and mutagenesis; alternatively a gene expression vector is employed. A vector of use according to the invention may be selected to accommodate a polypeptide coding sequence of a desired size, typically from 0.25 kilobase (kb) to 40 kb or more in length. A suitable host cell is transformed with the vector after in vitro cloning manipulations. Each vector contains various functional components, which generally include a cloning (or “polylinker”) site, an origin of replication and at least one selectable marker gene. If the given vector is an expression vector, it additionally possesses one or more of the following: an enhancer element, a promoter, transcription, termination and signal sequences, each positioned in the vicinity of the cloning site, such that they are operatively linked to the gene encoding a dual-specific ligand according to the invention.

Both cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

In one embodiment, a cloning or expression vector may contain a selection gene also referred to as selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, (e.g. ampicillin, neomycin, methotrexate or tetracycline), complement auxotrophic deficiencies, or supply critical nutrients not available in the growth media.

Since the replication of vectors encoding a dual-specific ligand according to the present invention is most conveniently performed in E. coli, an E. coli-selectable marker, for example, the β-lactamase gene that confers resistance to the antibiotic ampicillin, is of use. These can be obtained from E. coli plasmids, such as pBR322 or a pUC plasmid such as pUC18 or pUC19.

Expression vectors usually contain a promoter that is recognised by the host organism and is operably linked to the coding sequence of interest. Such a promoter may be inducible or constitutive. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Promoters for use in bacterial systems will also generally contain a Shine-Delgarno sequence operably linked to the coding sequence.

The possible vectors are expression vectors that enable the expression of a nucleotide sequence corresponding to a polypeptide library member. Thus, selection with the first and/or second antigen or epitope can be performed by separate propagation and expression of a single clone expressing the polypeptide library member or by use of any selection display system. As described above, the possible selection display system is bacteriophage display. Thus, phage or phagemid vectors may be used, (e.g., pIT1 or pIT2). Leader sequences useful in the invention include pelB, stII, ompA, phoA, bla and pelA. One example is phagemid vectors, which have an E. coli. origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector contains a β-lactamase gene to confer selectivity on the phagemid and a lac promoter upstream of an expression cassette that consists (N to C terminal) of a pelB leader sequence (which directs the expressed polypeptide to the periplasmic space), a multiple cloning site (for cloning the nucleotide version of the library member), optionally, one or more peptide tags (for detection), optionally, one or more TAG stop codon and the phage protein pIII. Thus, using various suppressor and non-suppressor strains of E. coli and with the addition of glucose, iso-propyl thio-β-D-galactoside (IPTG) or a helper phage, such as VCS M13, the vector is able to replicate as a plasmid with no expression, produce large quantities of the polypeptide library member only or produce phage, some of which contain at least one copy of the polypeptide-pIII fusion on their surface.

Construction of vectors encoding dual-specific ligands according to the invention employs conventional ligation techniques. Isolated vectors or DNA fragments are cleaved, tailored, and religated in the form desired to generate the required vector. If desired, analysis to confirm that the correct sequences are present in the constructed vector can be performed in a known fashion. Suitable methods for constructing expression vectors, preparing in vitro transcripts, introducing DNA into host cells, and performing analyses for assessing expression and function are known to those skilled in the art. The presence of a gene sequence in a sample is detected, or its amplification and/or expression quantified by conventional methods, such as Southern or Northern analysis, Western blotting, dot blotting of DNA, RNA or protein, in situ hybridisation, immunocytochemistry or sequence analysis of nucleic acid or protein molecules. Those skilled in the art will readily envisage how these methods may be modified, if desired.

### Skeletons

Skeletons may be based on immunoglobulin molecules or may be non-immunoglobulin in origin as set forth above. Each domain of a ligand (e.g, dual-specific ligand) may be a different skeleton. Possible immunoglobulin skeletons as herein defined includes any one or more of those selected from the following: an immunoglobulin molecule comprising at least (i) the CL (kappa or lambda subclass) domain of an antibody; or (ii) the CH1 domain of an antibody heavy chain; an immunoglobulin molecule comprising the CH1 and CH2 domains of an antibody heavy chain; an immunoglobulin molecule comprising the CH1 CH2 and CH3 domains of an antibody heavy chain; or any of the subset (ii) in conjunction with the CL (kappa or lambda subclass) domain of an antibody. A hinge region domain may also be included. For example, the ligand can comprise a heavy chain constant region of an immunoglobulin (e.g., IgG (e.g., IgG1, IgG2, IgG3, IgG4) IgM, IgA, IgD or IgE) or portion thereof (e.g., Fc portion) and/or a light chain constant region (e.g., C_{λ}, C_{κ}). For example, the ligand can comprise CH1 of IgG1 (e.g., human IgG1), CH1 and CH2 of IgG1 (e.g., human IgG1), CHL CH2 and CH3 of IgG1 (e.g., human IgG1), CH2 and CH3 of IgG1 (e.g., human IgG1), or CH1 and CH3 of IgG1 (e.g., human IgG1). Such combinations of domains may, for example, mimic natural antibodies, such as IgG or IgM, or fragments thereof, such as Fv, scFv, Fab or F(ab′)₂ molecules. Those skilled in the art will be aware that this list is not intended to be exhaustive.

### Protein Scaffolds

Each binding domain can comprise a protein scaffold and one or more CDRs (e.g., of the dAbs disclosed herein) which are involved in the specific interaction of the domain with one or more epitopes. In one embodiment, an epitope binding domain according to the present invention comprises three CDRs. Suitable protein scaffolds include any of those selected from the group consisting of the following: those based on immunoglobulin domains, those based on fibronectin, those based on affibodies, those based on CTLA4, those based on chaperones such as GroEL, those based on lipocallin and those based on the bacterial Fc receptors SpA and SpD. Those skilled in the art will appreciate that this list is not intended to be exhaustive. The binding domains can also comprise a protein scaffold that has a binding site that has binding specificity for a target (e.g., IL-4, IL-13), but does not contain one or more CDRs (e.g., of the dAbs disclosed herein). For example, the binding domain can be a protein scaffold that has a binding site that has binding specificity for a target selected from an affibody, an SpA domain, based on CTLA4, those based on chaperones such as GroEL, those based on lipocallin and those based on the bacterial Fc receptors SpA and SpD, an LDL receptor class A domain, an avimer (see, e.g., U.S. Patent Application Publication Nos. 2005/0053973, 2005/0089932, 2005/0164301).

### Therapeutic and Diagnostic Compositions and Uses

The invention provides compositions comprising the ligands of the invention and a pharmaceutically acceptable carrier, diluent or excipient, and therapeutic and diagnostic methods that employ the ligands or compositions of the invention. The ligands according to the method of the present invention may be employed in in vivo therapeutic and prophylactic applications, in vivo diagnostic applications and the like.

Therapeutic and prophylactic uses of ligands of the invention involve the administration of ligands according to the invention to a recipient mammal, such as a human. The ligands bind to targets with high affinity and/or avidity. In some embodiments, such as IgG-like ligands, the ligands can allow recruitment of cytotoxic cells to mediate killing of cancer cells, for example by antibody dependent cellular cytoxicity.

Substantially pure ligands of at least 90 to 95% homogeneity are possible for administration to a mammal, and 98 to 99% or more homogeneity is possible for pharmaceutical uses, especially when the mammal is a human. Once purified, partially or to homogeneity as desired, the ligands may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest. Treatment includes ameliorating symptoms associated with the disease, and also preventing or delaying the onset of the disease and also lessening the severity or frequency of symptoms of the disease.

For example, the ligands, of the present invention will typically find use in preventing, suppressing or treating disease states. For example, ligands can be administered to treat, suppress or prevent a chronic inflammatory disease, allergic hypersensitivity, cancer, bacterial or viral infection, autoimmune disorders (which include, but are not limited to, Type I diabetes, asthma, multiple sclerosis, rheumatoid arthritis, juvenile rheumatoid arthritis, psoriatic arthritis, spondylarthropathy (e.g., ankylosing spondylitis), systemic lupus erythematosus, inflammatory bowel disease (e.g., Crohn's disease, ulcerative colitis), myasthenia gravis and Behcet's syndrome, psoriasis, endometriosis, and abdominal adhesions (e.g., post abdominal surgery).

The ligands of the invention may be used to treat, suppress or prevent disease, such as an allergic disease, a Th2-mediated disease, IL-13-mediated disease, IL-4-mediated disease, and/or IL-4/IL-13-mediated disease. Examples of such diseases include, Hodgkin's disease, asthma, allergic asthma, atopic dermatitis, atopic allergy, ulcerative colitis, scleroderma, allergic rhinitis, COPD, idiopathic pulmonary fibrosis, chronic graft rejection, bleomycin-induced pulmonary fibrosis, radiation-induced pulmonary fibrosis, pulmonary granuloma, progressive systemic sclerosis, schistosomiasis, hepatic fibrosis, renal cancer, Burkitt lymphoma, Hodgkins disease, non-Hodgkins disease, Sezary syndrome, asthma, septic arthritis, dermatitis herpetiformis, chronic idiopathic urticaria, ulcerative colitis, scleroderma, hypertrophic scarring, Whipple's Disease, benign prostate hyperplasia, a lung disorder in which IL-4 receptor plays a role, condition in which IL-4 receptor-mediated epithelial barrier disruption plays a role, a disorder of the digestive system in which IL-4 receptor plays a role, an allergic reaction to a medication, Kawasaki disease, sickle cell disease, Churg-Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, cystic fibrosis, allergic bronchopulmonary mycosis, chronic obstructive pulmonary disease, bleomycin-induced pneumopathy and fibrosis, pulmonary alveolar proteinosis, adult respiratory distress syndrome, sarcoidosis, hyper IgE syndrome, idiopathic hypereosinophil syndrome, an autoimmune blistering disease, pemphigus vulgaris, bullous pemphigoid, myasthenia gravis, chronic fatigue syndrome, nephrosis).

The term “allergic disease” refers to a pathological condition in which a patient is hypersensitized to and mounts an immunologic reaction against a substance that is normally nonimmunogenic. Allergic disease is generally characterized by activation of mast cells by IgE resulting in an inflammatory response (e.g., local response, systemic response) that can result in symptoms as benign as a runny nose, to life-threatening anaphylactic shock and death. Examples of allergic disease include, but are not limited to, allergic rhinitis (e.g., hay fever), asthma (e.g., allergic asthma), allergic dermatitis (e.g., eczema), contact dermatitis, food allergy and urticaria (hives).

As used herein “Th2-mediated disease” refers to a disease in which pathology is produced (in whole or in part) by an immune response (Th2-type immune response) that is regulated by CD4⁺ Th2 T lymphocytes, which characteristically produce IL-4, IL-5, IL-10 and IL-13. A Th2-type immune response is associated with the production of certain cytokines (e.g., IL-4, IL-13) and of certain classes of antibodies (e.g., IgE), and is associate with humor immunity. Th2-meidated diseases are characterized by the presence of elevated levels of Th2 cytokines (e.g., IL-4, IL-13) and/or certain classes of antibodies (e.g., IgE) and include, for example, allergic disease (e.g., allergic rhinitis, atopic dermatitis, asthma (e.g., atopic asthma), allergic airways disease (AAD), anaphylactic shock, conjunctivitis), autoimmune disorders associated with elevated levels of IL-4 and/or IL-13 (e.g., rheumatoid arthritis, host-versus-graft disease, renal disease (e.g., nephritic syndrome, lupus nephritis)), and infections associated with elevated levels of IL-4 and/or IL-13 (e.g., viral, parasitic, fungal (e.g., C. albicans) infection).

Certain cancers are associated with elevated levels of IL-4 and/or IL-13 or associated with IL-4-induced and/or IL-13-induced cancer cell proliferation (e.g., B cell lymphoma, T cell lymphoma, multiple myeloma, head and neck cancer, breast cancer and ovarian cancer). These cancers can be treated, suppressed or prevented using the ligand of the invention.

Generally, the present ligands will be utilized in purified form together with pharmacologically appropriate carriers. Typically, these carriers include aqueous or alcoholic/aqueous solutions, emulsions or suspensions, and include saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition). A variety of suitable formulations can be used, including extended release formulations.

The ligand of the present invention may be used as separately administered compositions or in conjunction with other agents. The ligands can be used in combination therapy with existing IL-13 therapeutics (e.g., existing IL-13 agents (for example, anti-IL-13Rα1, IL-4/13 Trap, anti-IL-13) plus IL-4 dAb, and existing IL-4 agents (for example, anti-IL-4R, IL-4 Mutein, IL-4/13 Trap) plus IL-13 dAb) and IL-13 and IL-4 antibodies (for example, WO05/0076990 (CAT), WO03/092610 (Regeneron), WO00/64944 (Genetic Inst.) and WO2005/062967 (Tanox)). The ligands can be administered and or formulated together with one or more additional therapeutic or active agents. When a ligand is administered with an additional therapeutic agent, the ligand can be administered before, simultaneously with or subsequent to administration of the additional agent. Generally, the ligand and additional agent are administered in a manner that provides an overlap of therapeutic effect. Additional agents that can be administered or formulated with the ligand of the invention include, for example, various immunotherapeutic drugs, such as cylcosporine, methotrexate, adriamycin or cisplatinum, antibiotics, antimycotics, anti-viral agents and immunotoxins. For example, when the antagonist is administered to prevent, suppress or treat lung inflammation or a respiratory disease (e.g., asthma), it can be administered in conjuction with phosphodiesterase inhibitors (e.g., inhibitors of phosphodiesterase 4), bronchodilators (e.g., beta2-agonists, anticholinergerics, theophylline), short-acting beta-agonists (e.g., albuterol, salbutamol, bambuterol, fenoterol, isoetherine, isoproterenol, levalbuterol, metaproterenol, pirbuterol, terbutaline and tornlate), long-acting beta-agonists (e.g., formoterol and salmeterol), short acting anticholinergics (e.g., ipratropium bromide and oxitropium bromide), long-acting anticholinergics (e.g., tiotropium), theophylline (e.g. short acting formulation, long acting formulation), inhaled steroids (e.g., beclomethasone, beclometasone, budesonide, flunisolide, fluticasone propionate and triamcinolone), oral steroids (e.g., methylprednisolone, prednisolone, prednisolon and prednisone), combined short-acting beta-agonists with anticholinergics (e.g., albuterol/salbutamol/ipratopium, and fenoterol/ipratopium), combined long-acting beta-agonists with inhaled steroids (e.g., salmeterol/fluticasone, and formoterol/budesonide) and mucolytic agents (e.g., erdosteine, acetylcysteine, bromheksin, carbocysteine, guiafenesin and iodinated glycerol.

Other suitable co-therapeutic agents that can be administed with a ligand of the invention to prevent, suppress or treat asthma (e.g., allergic asthma), include a corticosteroid (e.g., beclomethasone, budesonide, fluticasone), cromoglycate, nedocromil, beta-agonist (e.g., salbutamol, terbutaline, bambuterol, fenoterol, reproterol, tolubuterol, salmeterol, fomtero), zafirlukast, salmeterol, prednisone, prednisolone, theophylline, zileutron, montelukast, and leukotriene modifiers.

The ligands of the invention can be coadministered with a variety of co-therapeutic agents suitable for treating diseases (e.g., a Th-2 mediated disease, IL-4-mediated disease, IL-13-mediated disease, IL-4 and IL-13-mediated disease, cancer), including cytokines, analgesics/antipyretics, antiemetics, and chemotherapeutics.

Cytokines include, without limitation, a lymphokine, tumor necrosis factors, tumor necrosis factor-like cytokine, lymphotoxin, interferon, macrophage inflammatory protein, granulocyte monocyte colony stimulating factor, interleukin (including, without limitation, interleukin-1, interleukin-2, interleukin-6, interleukin-12, interleukin-15, interleukin-18), growth factors, which include, without limitation, (e.g., growth hormone, insulin-like growth factor 1 and 2 (IGF-1 and IGF-2), granulocyte colony stimulating factor (GCSF), platelet derived growth factor (PGDF), epidermal growth factor (EGF), and agents for erythropoiesis stimulation, e.g., recombinant human erythropoietin (Epoetin alfa), EPO, a hormonal agonist, hormonal antagonists (e.g., flutamide, tamoxifen, leuprolide acetate (LUPRON)), and steroids (e.g., dexamethasone, retinoid, betamethasone, cortisol, cortisone, prednisone, dehydrotestosterone, glucocorticoid, mineralocorticoid, estrogen, testosterone, progestin).

Analgesics/antipyretics can include, without limitation, (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and the like).

Antiemetics can also be coadministered to prevent or treat nausea and vomiting, e,g., suitable antiemetics include meclizine hydrochloride, nabilone, prochlorperazine, dimenhydrinate, promethazine hydrochloride, thiethylperazine, scopolamine, and the like).

Chemotherapeutic agents, as that term is used herein, include, but are not limited to, for example antimicrotubule agents, (e.g., taxol (paclitaxel)), taxotere (docetaxel); alkylating agents (e.g., cyclophosphamide, carmustine, lomustine, and chlorambucil); cytotoxic antibiotics (e.g., dactinomycin, doxorubicin, mitomycin-C, and bleomycin; antimetabolites (e.g., cytarabine, gemcitatin, methotrexate, and 5-fluorouracil); antimiotics (e.g., vincristine vinca alkaloids (e.g., etoposide, vinblastine, and vincristine)); and others such as cisplatin, dacarbazine, procarbazine, and hydroxyurea; and combinations thereof.

Pharmaceutical compositions can include “cocktails” of various cytotoxic or other agents in conjunction with ligands of the present invention, or even combinations of ligands according to the present invention having different specificities, such as ligands selected using different target antigens or epitopes, whether or not they are pooled prior to administration.

The route of administration of pharmaceutical compositions according to the invention may be any suitable route, such as any of those commonly known to those of ordinary skill in the art. For therapy, including without limitation immunotherapy, the ligands of the invention can be administered to any patient in accordance with standard techniques. The administration can be by any appropriate mode, including parenterally, intravenously, intramuscularly, intraperitoneally, transdermally, intrathecally, intraarticularly, via the pulmonary route, or also, appropriately, by direct infusion (e.g., with a catheter). The dosage and frequency of administration will depend on the age, sex and condition of the patient, concurrent administration of other drugs, counterindications and other parameters to be taken into account by the clinician. Administration can be local (e.g., local delivery to the lung by pulmonary administration, (e.g., intranasal administration) or local injection directly into a tumor) or systemic as indicated.

The ligands of this invention can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate.

The compositions containing the ligands can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's health, but generally range from 0.005 to 5.0 mg of ligand per kilogram of body weight, with doses of 0.05 to 2.0 mg/kg/dose being more commonly used. For prophylactic applications, compositions containing the present ligands or cocktails thereof may also be administered in similar or slightly lower dosages, to prevent, inhibit or delay onset of disease (e.g., to sustain remission or quiescence, or to prevent acute phase). The skilled clinician will be able to determine the appropriate dosing interval to treat, suppress or prevent disease. When a ligand is administered to treat, suppress or prevent a disease, it can be administered up to four times per day, twice weekly, once weekly, once every two weeks, once a month, or once every two months, at a dose of, for example, about 10 mg/kg to about 80 mg/kg, about 100 mg/kg to about 80 mg/kg, about 1 mg/kg to about 80 mg/kg, about 1 mg/kg to about 70 mg/kg, about 1 mg/kg to about 60 mg/kg, about 1 mg/kg to about 50 mg/kg, about 1 mg/kg to about 40 mg/kg, about 1 mg/kg to about 30 mg/kg, about 1 mg/kg to about 20 mg/kg, about 1 mg/kg to about 10 mg/kg, about 10 μg/kg to about 10 mg/kg, about 10 μg/kg to about 5 mg/kg, about 10 μg/kg to about 2.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg. In particular embodiments, the ligand is administered to treat, suppress or prevent a chronic allergic disease once every two weeks or once a month at a dose of about 10 μg/kg to about 10 mg/kg (e.g., about 10 μg/kg, about 100 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg.)

In particular embodiments, the ligand is administered to treat, suppress or prevent asthma each day, every two days, once a week, once every two weeks or once a month at a dose of about 10 mg/kg to about 10 mg/kg (e.g., about 10 μg/kg, about 100 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg). The ligand can also be administered at a daily dose or unit dose (e.g., to treat, suppress or prevent asthma) at a daily dose or unit dose of about 10 mg, about 9 mg, about 8 mg, about 7 mg, about 6 mg, about 5 mg, about 4 mg, about 3 mg, about 2 mg or about 1 mg.

In particular embodiments, the ligand of the invention is administered at a dose that provides saturation of IL-4 and/or IL-13 or a desired serum concentration in vivo. The skilled physician can determine appropriate dosing to achieve saturation, for example by titrating ligand and monitoring the amount of free binding sites on IL-4 and/or IL-13 or the serum concentration of ligand. Therapeutic regiments that involve administering a therapeutic agent to achieve target saturation or a desired serum concentration of agent are common in the art.

Treatment or therapy performed using the compositions described herein is considered “effective” if one or more symptoms are reduced (e.g., by at least 10% or at least one point on a clinical assessment scale), relative to such symptoms present before treatment, or relative to such symptoms in an individual (human or animal model) not treated with such composition or other suitable control. Symptoms will obviously vary depending upon the disease or disorder targeted, but can be measured by an ordinarily skilled clinician or technician. Such symptoms can be measured, for example, by monitoring the level of one or more biochemical indicators of the disease or disorder (e.g., levels of an enzyme or metabolite correlated with the disease, affected cell numbers, etc.), by monitoring physical manifestations (e.g., inflammation, tumor size, etc.), or by an accepted clinical assessment scale, for example, Juniper's Asthma Qualtiy of Life Questionnaire (American Thoracic Society's 32 item assessment evaluates the quality of life with respect to activity limitations, symptoms, emotional function and exposure to environmental stimuli; Juniper, et. al., “Health-related Quality of Life in Moderate Asthma,” Chest, 116:1297-1303 (1999).), the Expanded Disability Status Scale (for multiple sclerosis), the Irvine Inflammatory Bowel Disease Questionnaire (32 point assessment evaluates quality of life with respect to bowel function, systemic symptoms, social function and emotional status-score ranges from 32 to 224, with higher scores indicating a better quality of life), the Quality of Life Rheumatoid Arthritis Scale, or other accepted clinical assessment scale as known in the field. A sustained (e.g., one day or more, in one embodiment longer) reduction in disease or disorder symptoms by at least 10% or by one or more points on a given clinical scale is indicative of “effective” treatment. Similarly, prophylaxis performed using a composition as described herein is “effective” if the onset or severity of one or more symptoms is delayed, reduced or abolished relative to such symptoms in a similar individual (human or animal model) not treated with the composition.

A composition containing ligands according to the present invention may be utilized in prophylactic and therapeutic settings to aid in the alteration, inactivation, killing or removal of a select target cell population in a mammal. In addition, the ligands and selected repertoires of polypeptides described herein may be used extracorporeally or in vitro selectively to kill, deplete or otherwise effectively remove a target cell population from a heterogeneous collection of cells. Blood from a mammal may be combined extracorporeally with the ligands, e.g. antibodies, cell-surface receptors or binding proteins thereof whereby the undesired cells are killed or otherwise removed from the blood for return to the mammal in accordance with standard techniques.

### EXAMPLES

Reference is made to Examples 1 to 4 in WO2007/0858152A2 for general methodologies that are applicable to the present invention, including the assays set out in Example 2 of W02007/0858152A2, which can be used with the present invention. These disclosures are incorporated herein by reference as though repeated verbatim herein.

### Example 1

### Ligands that Bind IL-13

### Potencies of anti-IL-13 dAbs DOM10-53-474 and DOM10-275-78 HEK cell assay

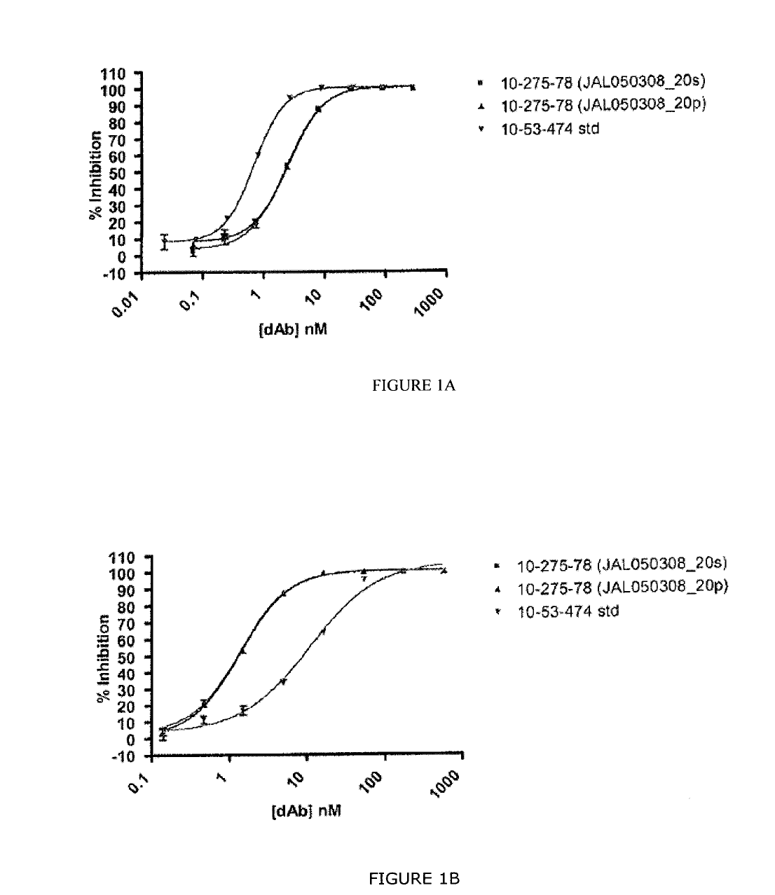

This assay uses HEK293 cells stably transfected with the STAT6 gene and the SEAP (secreted embryonic alkaline phosphatase) reporter gene (Invivogen, San Diego). Upon stimulation with IL-13 SEAP is secreted into the supernatant which is measured using a colorimetric method. Soluble dAbs were tested for their ability to block IL-13 signalling via the STAT6 pathway. Briefly, the dAb is pre-incubated with 6 ng/ml recombinant IL-13 (GSK) for one hour then added to 50000 HEKSTAT6 cells in DMEM (Gibco, Invitrogen Ltd, Paisley, UK) in a tissue culture microtitre plate. The plate is incubated for 24 hours at 37° C. 5% CO₂. The culture supernatant is then mixed with QuantiBlue (Invivogen) and the absorbance read at 640 nm. Anti-IL-13 dAb activity causes a decrease in STAT6 activation and a corresponding decrease in A₆₄₀ compared to IL-13 stimulation. (FIG. 1)

TABLE 1

| | 10-53-474 | 10-275-78 | 10-275-94 | 10-275-99 | 10-275-100 | 10-275-101 |
|---|---|---|---|---|---|---|
| | EC50 (nM) | EC50 (nM) | EC50 (nM) | EC50 (nM) | EC50 (nM) | EC50 (nM) |
| HEK assay | 0.63 | 2.5 | 2.3 | 2.8 | 2.8 | 3.6, 2.0 |
| hIL-13 | (n = 13) | (n = 7) | | | | |
| HEK assay | 11.1 | 1.4 | 2.0 | 2.0 | 2.5 | 1.8 |
| cIL-13 | (n = 10) | (n = 7) | | | | |

### Sandwich ELISA

### IL-13 Sandwich ELISA

A MAXISORP™ plate (high protein binding ELISA plate, Nunc, Denmark) was coated overnight with 2.5 μg/ml coating antibody (Module Set, Bender MedSystems, Vienna, Austria), then washed once with 0.05% (v/v) Tween 20 in PBS before blocking with 0.5% (w/v) BSA 0.05% (v/v) Tween 20 in PBS. The plates were washed again before the addition of 25 pg/ml IL-13 (Bender MedSystems) mixed with a dilution series of DOM10 dAb (i.e., an anti-IL-13 dAb) or IL-13. The plates were washed again before binding of IL-13 to the capture antibody was detected using biotin conjugated detection antibody (Module Set, Bender Medsystems), followed by peroxidase labelled Streptavidin (Module Set, Bender MedSystems). The plate was then incubated with TMB substrate (KPL, Gaithersburg, USA), and the reaction was stopped by the addition of HCl and the absorbance read at 450 nm. Anti-IL-13 dAb activity caused a decrease in IL-13 binding and therefore a decrease in absorbance compared with the IL-13 only control. Table 2 shows the results of the ELISA.

TABLE 2

| | 10-53-474 (EC₅₀) |
|---|---|
| IL-13 | 0.023 nM (n = 23) |

### BIACORE® Off-Rate Screening

A streptavidin coated SA chip (Biacore) was coated with approximately 100 RU of biotinylated human IL-13 (R&D Systems, Minneapolis, USA) or cynomolgous IL-13 (Produced in-house). dAbs were serially diluted in HBS-EP running buffer. 50 to 100 ul of the diluted supernatant was injected (kininject) at 50 ul/min flow rate, followed by a 5 minute dissociation phase. Association and dissociation off-rates and constants were calculated using BIAevaluation software v4.1 (Biacore). Table 3 shows the KD (K_{off}/Kₒₙ).

TABLE 3

| | DOM10-53-474 | DOM10-275-78 |
|---|---|---|
| | (nM) | (nM) |
| Biacore hIL-13 | 0.028 | 0.072-0.1 |
| Biacore cIL-13 | 2.0 | 0.32-0.75 |

Binding to variant IL-13 (R130Q)

Genetic variants of IL-13, of which R130Q is a common variant, have been associated with an increased risk for asthma (Heinzmann et al. Hum Mol. Genet. (2000) 9549-59) and bronchial hyperresponsiveness (Howard et al., Am. J. Resp. Cell Molec. Biol. (2001) 377-384). Therefore it is desirable for the anti-IL-13 dAb to also have binding affinity for this variant of the cytokine. DOM10-53-474 bound IL-13 (R130Q) and inhibited IL-13 (R130Q) stimulated proliferation in two cell assays (TF-1 & Hek-Stat6).

TABLE 4
(DOM10-53-474)

| Cell Assay | EC50 nM |
|---|---|
| Hek-Stat6 (variant hIL-13 stimulation = | 0.273 (n = 4) |
| 3 ng/ml) | |
| TF-1 (variant hIL-13 stimulation = | 0.133 (n =3) |
| 5 ng/ml) | |

### Agonistic Activity

To determine whether DOM10-53-474 binds non-target proteins, and to ensure that no undesired cytokines/interferons are released due to agonistic activity of the dAb, DOM10-53-474 was tested for agonistic activity in a human blood assay. Each sample was titrated from 1 μM to 10 nM of DOM10-53-474 and tested in two donors, A & B. The assay was set up in duplicate (a & b) and the meso scale discovery (MSD) was performed in duplicate. The nil wells contained blood alone, (i.e. no dAb added), there were 8 nil wells for donor A and 4 for donor B. The cytokines assayed were IL-8, IL-6, TNFα, IL-10, IL-1β, IL-12p70 and IFNγ. No agonistic activity was seen with respect to IL-6, TNFα, IL-10, IL-β, IL-12p70 or IFNγ. There was a little IL-8 production at the 1 μM concentration but this was very low.

### SEC-MALLS

The in-solution properties of dAb proteins were determined by an initial separation on SEC (size exclusion chromatography; TSKgel G2000/3000SWXL, Tosoh Biosciences, Germany; BioSep-SEC-S2000/3000, Phenomenex, Calif., USA) and subsequent on-line detection of eluting proteinaceous material by UV (Abs280 nm), R1 (refractive index) and light scattering (laser at 685 nm). The proteins were at an initial concentration of 2 mg/mL for DOM10-275-78 and 1.4 mg/ml for DOM10-53-474, as determined by absorbance at 280 nm, and visually inspected for impurities by SDS-PAGE. The homogeneity of samples to be injected was usually >90%. 100 uL were injected onto the SEC column. The protein separation on SEC was performed at 0.5 mL/min for 45 minutes. PBS (phosphate buffered saline±10% EtOH) was used as mobile phase. The ASTRA software (Wyatt Inc; CA; USA) integrated the signals of all three detectors and allowed for the determination of the molar masses in kDa of proteins from ‘first physical principles’. Inter-run variations and data quality was assessed by running a positive control of known in-solution state with every sample batch.

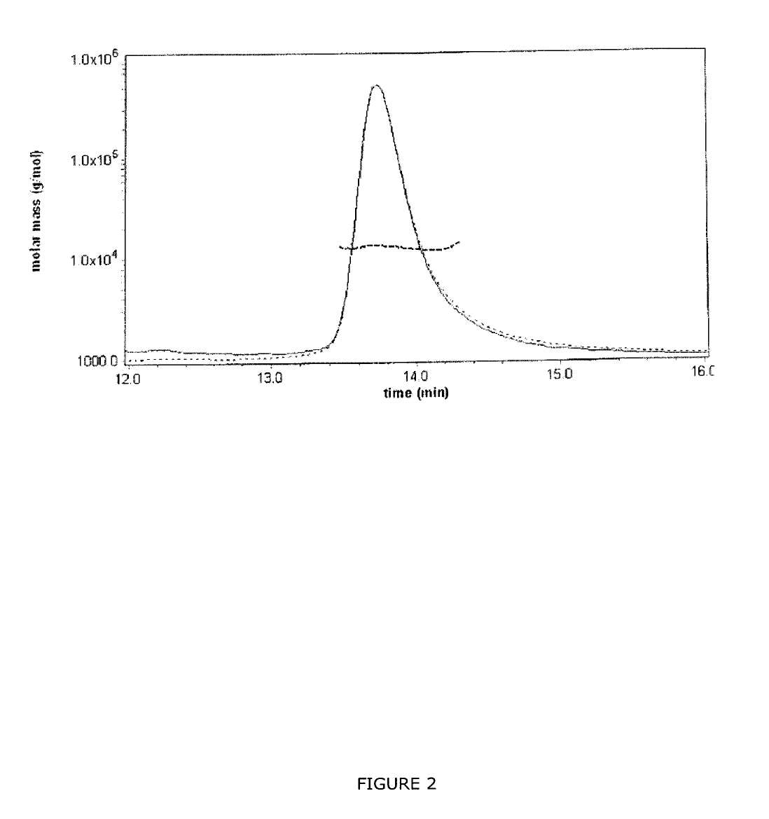

For some DOM10-53 clones no reliable solution state could be assigned because the molecules bound aspecifically to the column matrix or could not be resolved using the size exclusion column. For these cases where the solution state was reliable (i.e. DOM10-53-474 and DOM10-275-78) it was shown that the DOM10-275-78 molecule is mostly a monomer in solution and 90% is eluted from the column (FIG. 2), and that for the DOM10-53-474 molecule the majority of the protein is clear monomer (FIG. 3). DOM10-53-474 eluted as a single peak with the molar mass defined as 13 kDa in the right part of the peak (monomer) but creeping up over the left part of the peak up to 18 kDa, indicating some degree of rapid self association (average mass shown in the table is 14 kDa).

### DSC

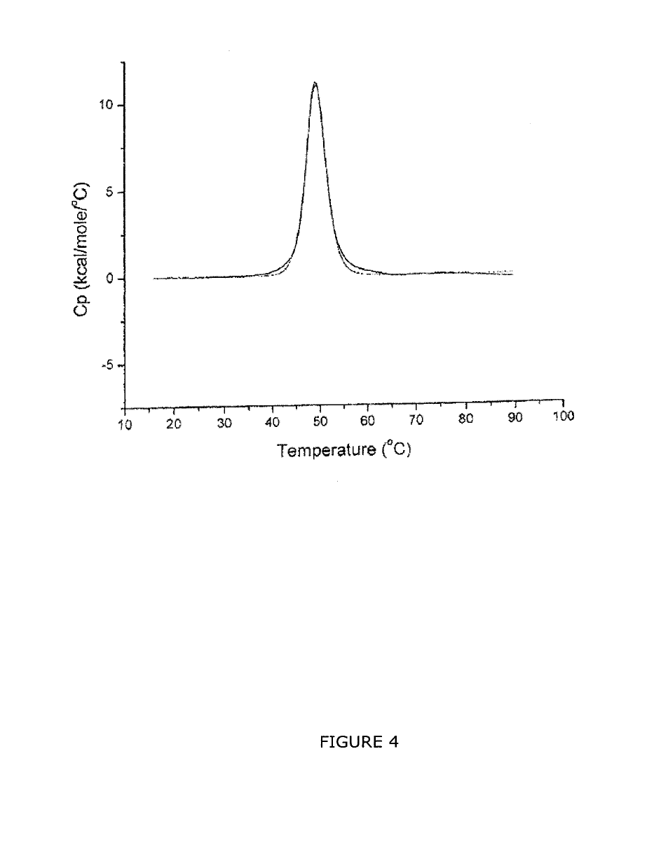

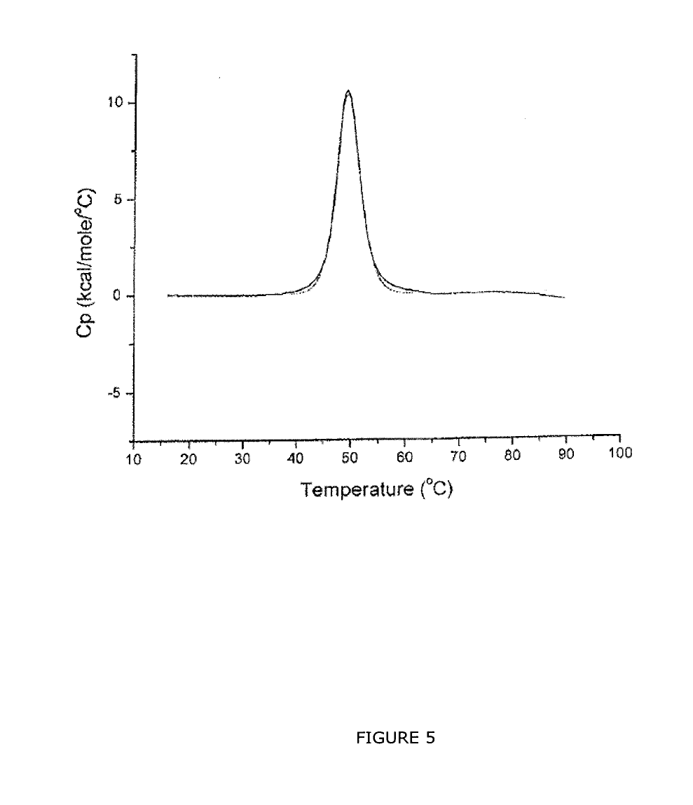

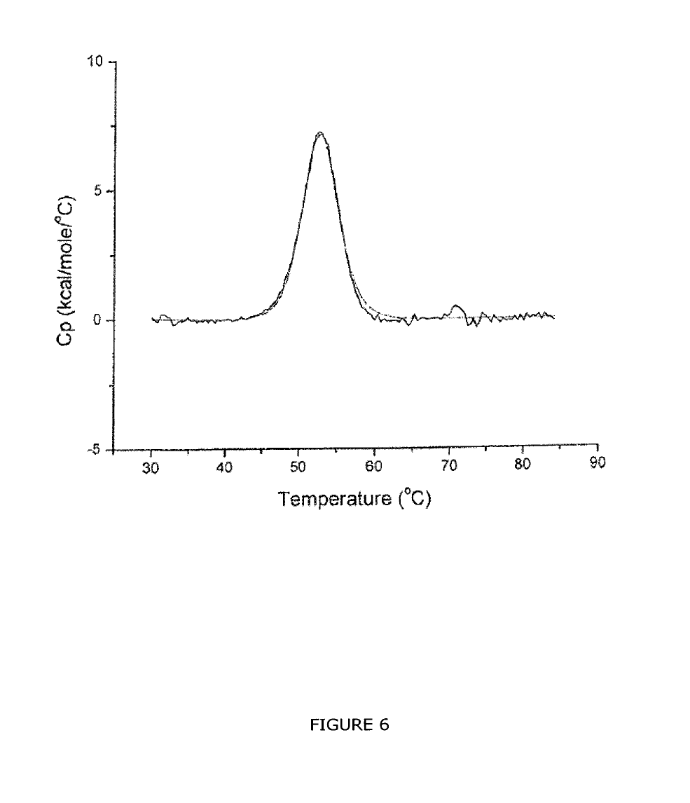

DOM10-275-78 protein was supplied in both PBS buffer (phosphate buffered saline) filtered to yield a concentration of 2 mg/ml, and in 50 mM potassium phosphate buffer pH7.4 at 2 mg/ml. Concentrations were determined by absorbance at 280 nm. PBS buffer and potassium phosphate buffer were used as a reference for the respective samples. DSC was performed using capillary cell microcalorimeter VP-DSC (Microcal, Mass., USA), at a heating rate of 180° C./hour. A typical scan usually was from 25-90° C. for both the reference buffer and the protein sample. After each reference buffer and sample pair, the capillary cell was cleaned with a solution of 1% Decon in water followed by PBS. Resulting data traces were analysed using Origin 7 Microcal software. The DSC trace obtained from the reference buffer was subtracted from the sample trace. The resultant traces are shown in FIGS. 4 AND 5. The precise molar concentration of the sample was entered into the data analysis routine to yield values for apparent Tm, enthalpy (ΔH) and van't Hoff enthalpy (ΔHv) values. Typically data were fitted to a non-2-state model. The DSC experiments showed that some DOM10 molecules (e.g. 10-53-474 (SEQ ID NO:1), FIG. 6, have higher melting temperatures compared to others (e.g. 10-275-78). Such properties are indicative of increased stability and indicate superior suitability, for example, for pulmonary delivery.

TABLE 5

| Molecule | Apparent Tm (° C.) |
|---|---|
| DOM10-274-78 in PBS | 49.4 |
| DOM10-275-78 in | 49.8 |
| potassium phosphate | |
| DOM10-53-474 in PBS | 54.0 |

The unfolding of DOM10-53-474 protein is irreversible, and therefore apparent Tm might be lower than the melting temperature due to some irreversible steps in the unfolding mechanism taking place before the melting point.

### Solubility

Liquid formulations that contain high dAb concentrations are desirable for certain purposes. For example, proteins delivered therapeutically via a nebulising device may need to be at higher concentrations than would be expected for systemic delivery because not all the nebulised protein will be inhaled nor deposited in the lung. Volumes administered are also limited by the size of the reservoir in the nebuliser of interest. To this end, the solubility of both DOM10-53-474 and DOM10-275-78 was measured to determine the maximum concentration that could be achieved before incurring protein losses through aggregation and precipitation.

The proteins of a known starting concentration in PBS, determined by measuring absorbance at 280 nm, and of a known volume were each applied to a Vivaspin 20 centrifugal concentrating device, with a PES membrane of MWCO 3,000Da (Vivasciences) and spun in a benchtop centrifuge at 4,000 g for time intervals of between 10 and 30 mins. Ten minute time periods were used initially and these were incremented as the protein became more concentrated in order to obtain the desired reduction in volume.

After each spin the protein was removed from the device, the volume measured to the nearest 50 μl using pipettes and the concentration determined. Concentration determination was performed using the absorbance reading obtained by subtracting the absorbance measured at 320 nm from the absorbance measured at 280 nm after the sample had been centrifuged at 16,000 g to remove any precipitate.

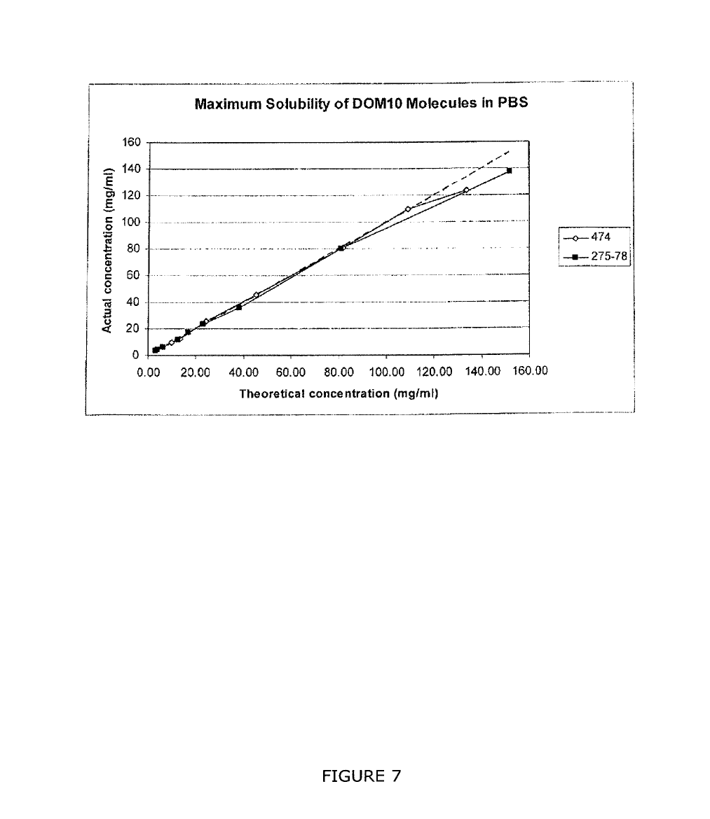

The experimental concentration was plotted against the theoretical concentration at that volume, and the maximum solubility was taken as the point at which experimental concentration diverged from theoretical as shown in FIG. 7.

For both proteins a concentration of 100 mg/ml was achieved before divergence and actual protein recovery was approximately 100% of the start material.

### Nebulisation of DOM10-53-474

The nebulising device can nebulise the dAb solution into droplets, only some of which will fall within the requisite size range for pulmonary deposition (1-5 μm). The particle size of the aerosol particles were analysed by laser light scattering using the Malvern Spraytek. Two post-nebulisation samples were collected i) protein solution which remained in the reservoir and ii) aerosolized protein collected by condensation. The parameters measured to assess the nebulisation process were i) Respirable fraction−% of particle in 1-5 μm size range, this is important to determine how much dAb will reach the deep lung; ii) Particle size distribution (psd) of dAb; iii) Mean median aerodynamic diameter (MMAD)—average droplet size of nebulised dAb solution within psd. The stability of the dAb to the nebulisation process was assessed by comparing pre- and post nebulisation samples using a variety of methods, i) Size Exclusion Chromatography (SEC)—which demonstrates whether the nebulisation process caused aggregation of the dAb; ii) Sandwich ELISA for binding to hIL-13.

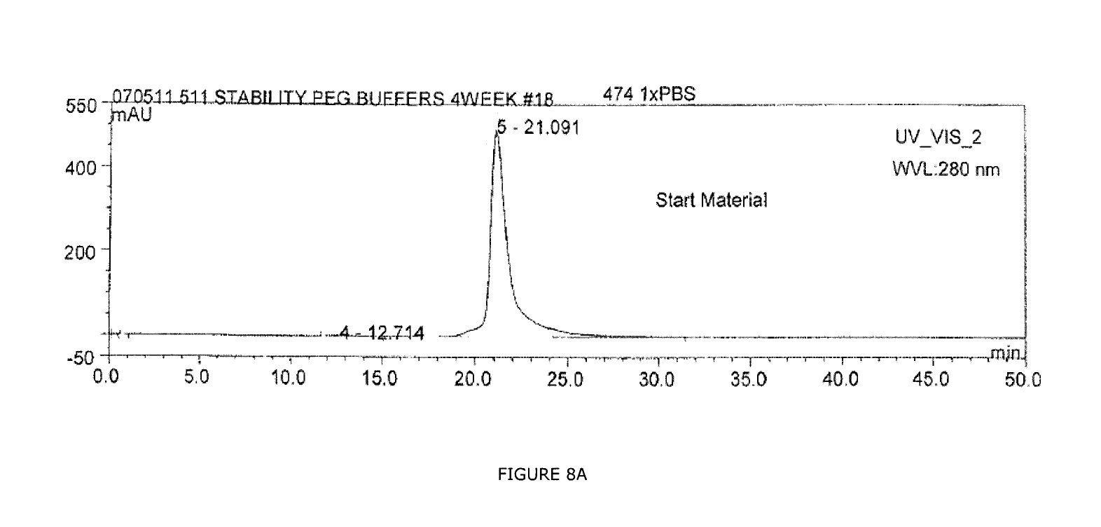

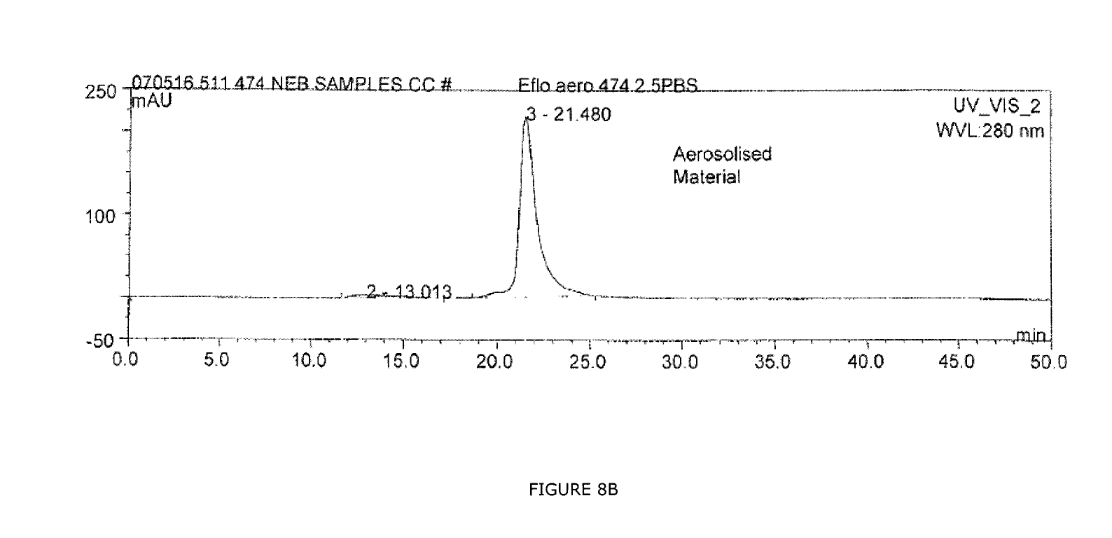

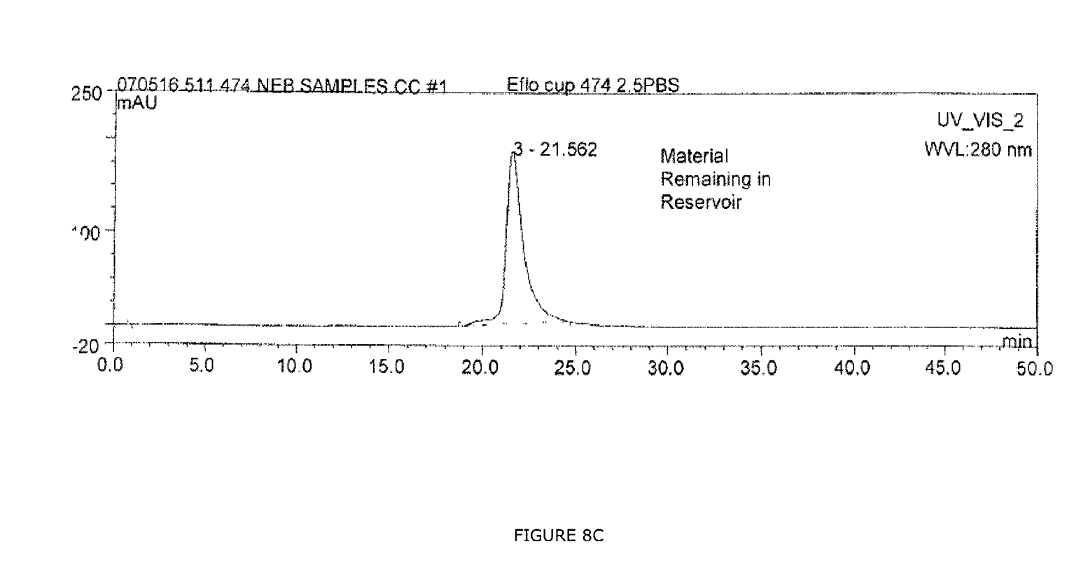

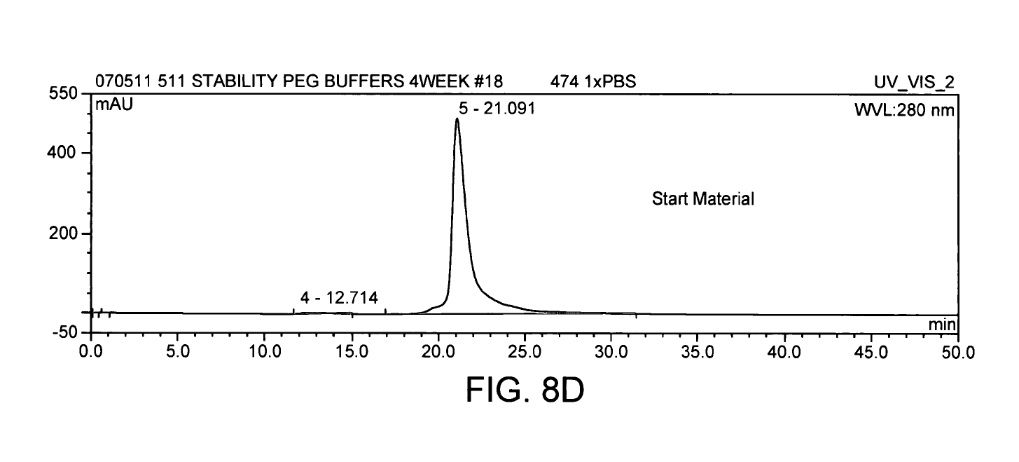

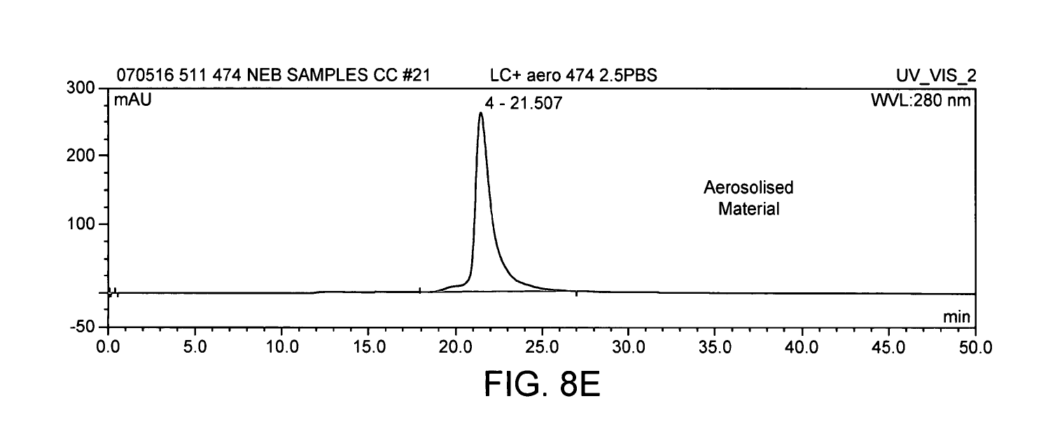

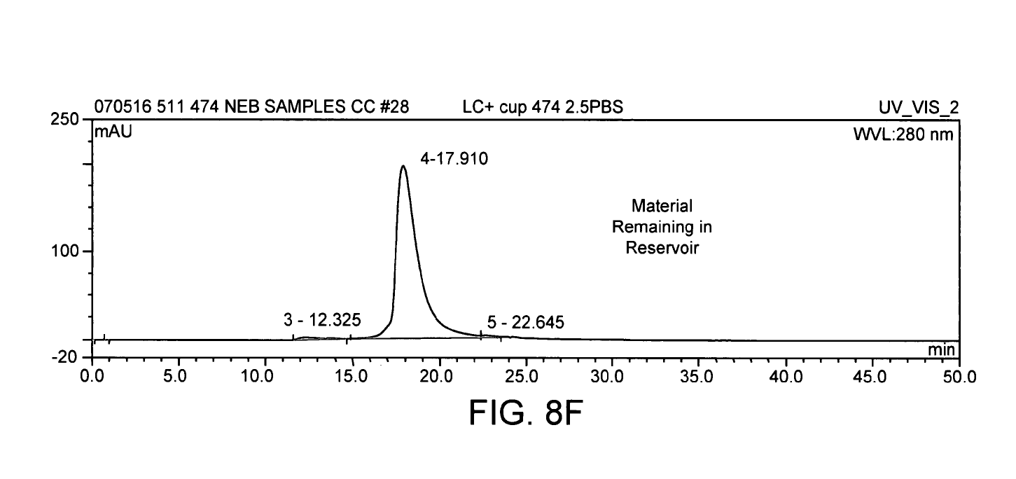

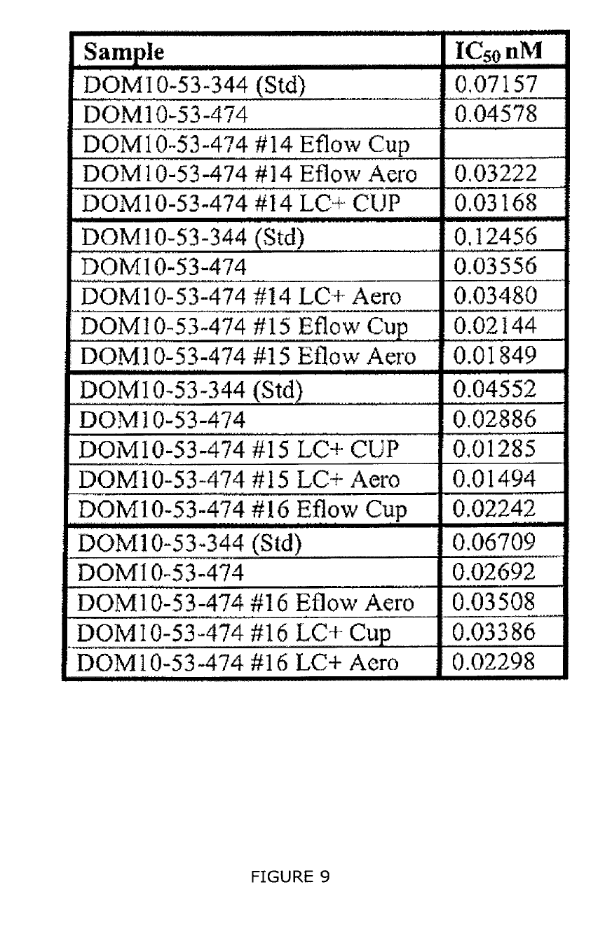

The nebulisation properties of DOM10-53-474 were investigated using both a jet nebuliser (LC+, Pari) and a vibrating mesh nebuliser (E-flow, Pari). DOM10-53-474 protein was tested in both PBS buffer (phosphate buffered saline) at a concentration of 2.6 mg/ml, and in 25 mM sodium phosphate buffer pH7.5, 7% (v/v) PEG1000, 1.2% (w/v) sucrose at 2.3 and 4.7 mg/ml. Nebulisation was performed for approximately 3 minutes. 100 uL of protein samples (diluted to 1 mg/mL) were injected onto the SEC (TSKgel G2000SWXL, Tosoh Biosciences, Germany) column. The protein separation on SEC was performed at 0.5 mL/min for 45 minutes. PBS (phosphate buffered saline)+10% EtOH was used as mobile phase. The detection of eluting proteinaceous material was carried by on-line detection by UV (Abs 280 nm & 215 nm). The SEC profile of the pre- and two post-nebulisation samples were identical; no peaks indicative of aggregation were seen post nebulisation, FIGS. 8A-F. The samples were analysed for binding to hIL-13 and the potency was shown to be unaffected by nebulisation, FIG. 9. The optimum MMAD is 3 μm and for deep lung delivery the desirable respirable fraction is the highest percentage of particles <5 μm. The LC+ (Pari) Jet nebuliser gives the better MMAD: MMAD values are lower when the buffer contains PEG; MMAD decreases as protein concentration increases. The LC+ (Pari) Jet nebuliser gives the higher %<5 μm: higher %<5 μm values are obtained when the buffer contains PEG; %<5 μm also increases as protein concentration increases.

TABLE 6

| | eFlow Rapid | | Pari LC + | |
|---|---|---|---|---|
| | MMAD | | MMAD | |
| Formulation | (um) | % < 5 um | (um) | % < 5 um |
| 25 mM NaPhosphate pH 7.5, 7% | 4.26 | 60.6% | 3.98 | 61.2% |
| PEG 1000, 1.2% Sucrose, | | | | |
| 2.3 mg/ml | | | | |
| 25 mM NaPhosphate pH 7.5, 7% | 4.10 | 63.8% | 3.66 | 66.5% |
| PEG 1000, 1.2 % Sucrose | | | | |
| 4.7 mg/ml | | | | |
| 10-53-474, PBS, | 5.20 | 47.9% | 4.43 | 56.6% |
| 2.6 mg/ml | | | | |

### Downstream Processing and Purity Obtained

A traditional method for initial capture and purification of antibodies and antibody fragments from fermenter supernatants or periplasmic fractions is using Protein A immobilised on an inert matrix. As an affinity chromatography step this has the advantage of good protein recovery and high (e.g. ˜90%) level of purity. However, there are some disadvantages. As with all forms of affinity chromatography some of the ligand can be leached from the column support matrix during the elution phase, Protein A is known to be a potential immunogen. Therefore, if Protein A is used, then any residual Protein A, leached from the column, should be removed or reduced as far as possible in subsequent chromatography steps.

### DOM10-275-78 Purification

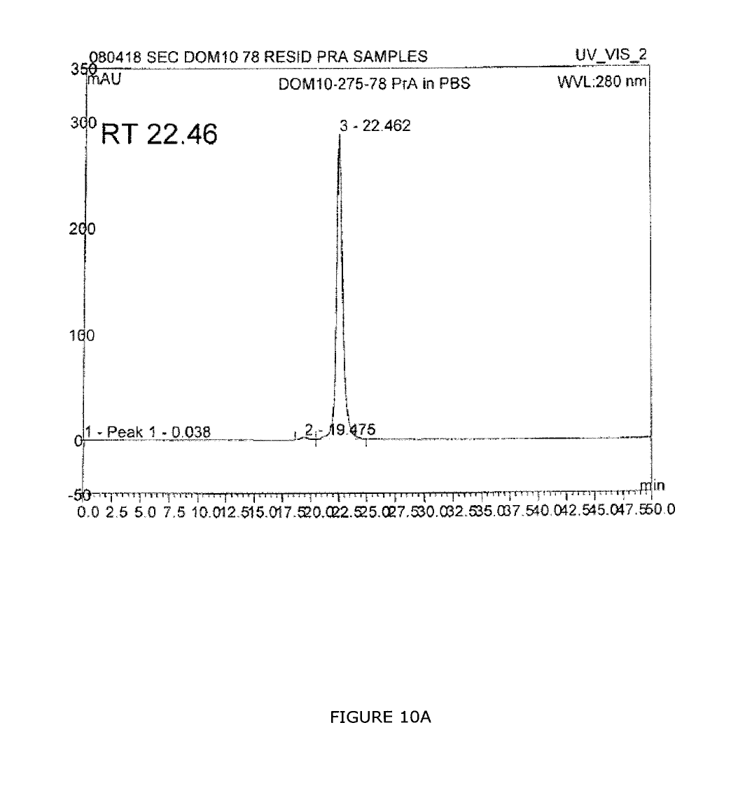

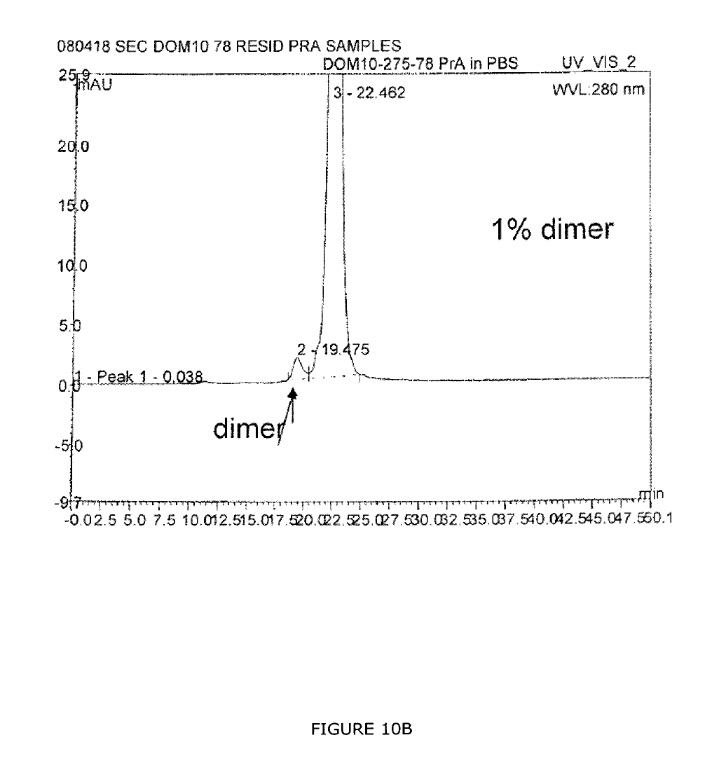

The initial capture step for either fermenter supernatants or periplasmic fraction containing DOM10-275-78 was by direct loading onto Protein A Streamline resin (GE Healthcare) equilibrated in PBS. The resin was washed with 2-5 column volumes of PBS before eluting the protein with 4 column volumes of 0.1M Glycine pH3.0. At this stage the eluted protein was approximately 99% pure, containing approximately 1% of dimeric DOM10-275-78 as measured by SEC and is shown in FIG. 10. Protein recovery was virtually 100%. Residual PrA was measured using a PrA ELISA kit (Cygnus, #F400) and was determined to be between 50 to 200 ppm.

### Residual PrA Removal

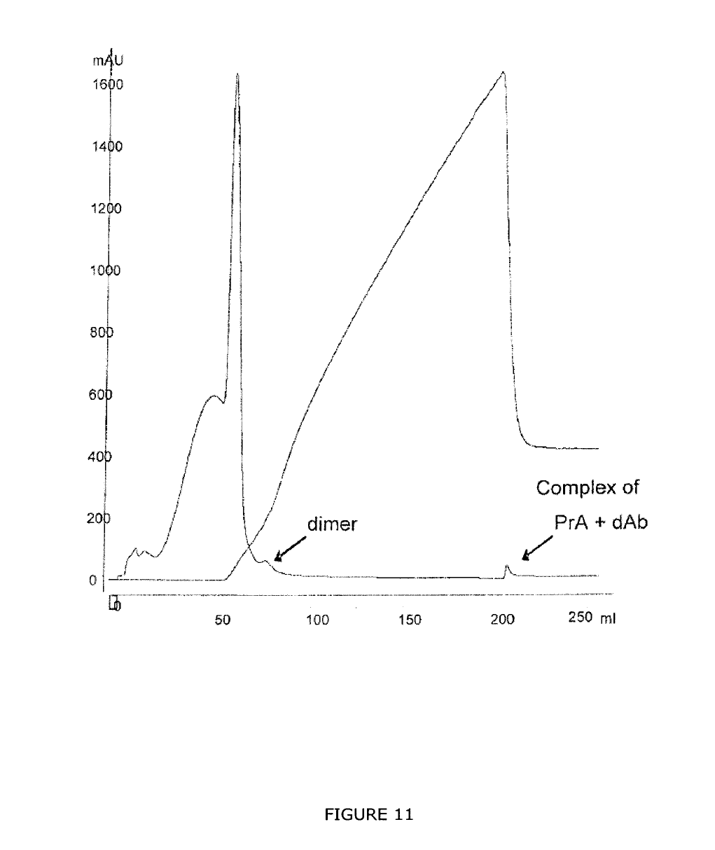

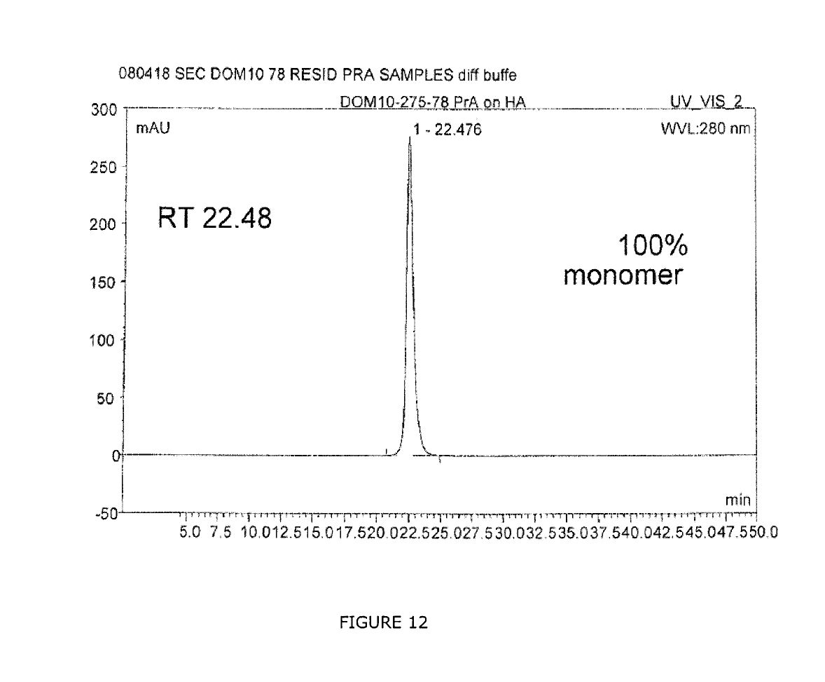

The residual PrA was reduced using two further chromatographic steps. The eluate from the PrA step was pH adjusted to pH6.5 using 1M Tris pH8.0 and prepared for purification on hydroxyapatite type II by addition of 1% (v/v) 0.5M sodium phosphate 016.5 resulting in a final phosphate concentration of 5 mM. The PrA eluate was applied to the column which had been equilibrated with 5 mM phosphate pH6.5 and the DOM10-275-78 monomer eluted in the flow through. The dimer was bound to the column and eluted at the start of a salt gradient which was applied after the DOM10-275-78 had been recovered. The gradient ran from 0 to 1M NaCl in 5 mM phosphate pH6.5 over 30 column volumes. It was expected that the PrA would elute in this gradient although amounts were too small to be able to see by absorbance on the chromatogram. Complexes of PrA with the DOM10-275-78 eluted after the salt gradient when a 500 mM phosphate pH6.5 wash was applied to the column. An example of a typical chromatogram is shown in FIG. 11. The recovery of DOM 10-275-78 monomer after this stage was measured as 74% based on absorbance at 280 nm and the purity was 100% as measured by SEC which is shown in FIG. 12. The residual protein levels were measured and were found to have been reduced to between 0.4 and 0.56 ppm (parts per million i.e. ng/mg).

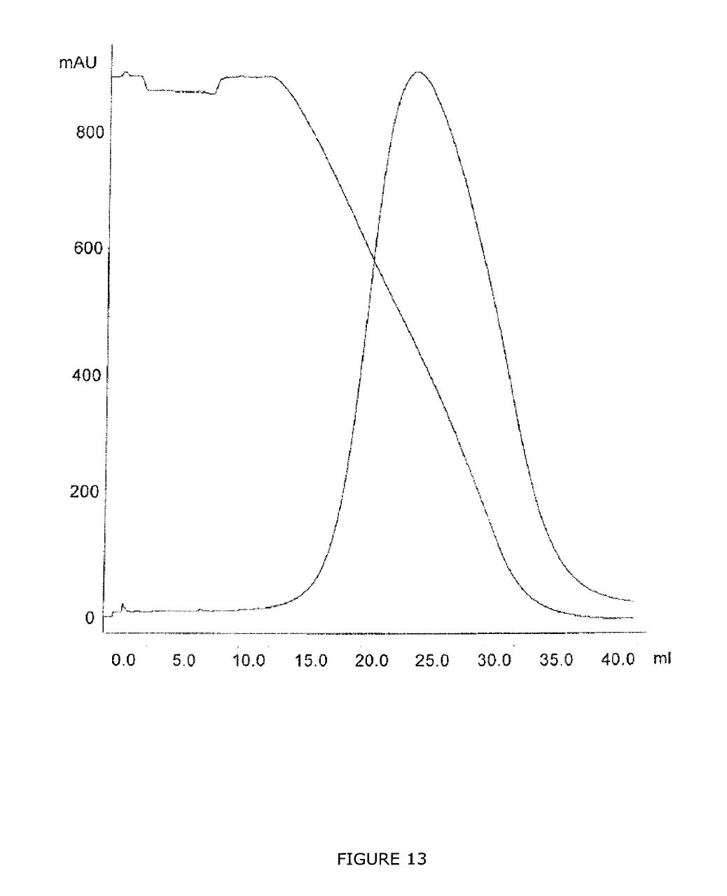

A further purification step was introduced to reduce the residual PrA even further. The eluate pool from the hydroxyapatite column was directly applied to a phenyl (HIC) column (GE Healthcare) after addition of NaCl to a final concentration of 2M. The column had been equilibrated with 25 mM phosphate pH7.4 plus 2M NaCl. The protein was eluted with a gradient from 2M NaCl to no salt over 20 column volumes as shown in the chromatogram in FIG. 13. After this step the residual PrA levels were reduced to between 0.15 to 0.19 ppm and the protein recovery was measured by absorbance at 280 nm as being 80%.

### Example 6

### Codon Optimization of Select Anti-IL-13 Dabs

Two anti-IL-13 dAbs were selected for codon optimization, DOM10-53-474 and DOM10-275-78. DOM10-53-474 was optimized for both E. coli expression (once) and Pichia pastoris soluble expression (twice). DOM10-275-78 was optimized once for E. coli expression.

The theoretical minimum percent identity of a codon optimised sequence to the wild-type dAb (i.e. maximimising the number of nucleotide changes within each degenerate codon to still encode the same amino acid sequence) for DOM10-53-474 is 57.6% and for DOM10-53-78 is 54.6%.

The actual percent identity for DOM10-53-474 optimized for E. coli expression (SEQ ID NO:3) was 79.0% sequence identity to wild-type DOM10-53-474. The actual percent identity for DOM10-53-474 optimized for Pichia pastoris soluble expression was 75.7% (SEQ ID NO:4) and 75.4% (SEQ ID NO:5).

The actual percent identity for DOM10-275-78 optimized for E. coli expression (SEQ ID NO:16) is 75.2%.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

### Sequence Correlation Table

The sequences below are presented in prior applications (number in the first bracket) and also in the present application (number in second bracket).

Amino Acid sequences:

DOM10-53-474

(SEQ ID NO:2369 in WO2007/085815A2), (SEQ ID NO: 1)

DOM 10-275-78

(SEQ ID NO:2456 in U.S. Ser. No. 12/152,903 & 12/397,826), (SEQ ID NO:6)

DOM10-275-94

(SEQ ID NO:2457 in U.S. Ser. No. 12/152,903 & 12/397,826), (SEQ ID NO:7)

DOM10-275-99

(SEQ ID NO:2458 in U.S. Ser. No. 12/152,903 & 12/397,826), (SEQ ID NO:8)

DOM10-275-100

(SEQ ID NO:2459 in U.S. Ser. No. 12/152,903 & 12/397,826), (SEQ ID NO:9)

DOM10-275-101

(SEQ ID NO:2460 in U.S. Ser. No. 12/152,903 & 12/397,826), (SEQ ID NO:10)

Nucleotide Sequences:

DOM10-53-474

(SEQ ID NO:2105 in WO2007/085815A2), (SEQ ID NO: 2)

DOM10-275-78

(SEQ ID NO:2464 in U.S. Ser. No. 12/152,903 & 12/397,826), (SEQ ID NO:11)

DOM10-275-94

(SEQ ID NO:2465 in U.S. Ser. No. 12/152,903 & 12/397,826), (SEQ ID NO:12)

DOM10-275-99

(SEQ ID NO:2466 in U.S. Ser. No. 12/152,903 & 12/397,826), (SEQ ID NO:13)

DOM10-275-100

(SEQ ID NO:2467 in U.S. Ser. No. 12/152,903 & 12/397,826), (SEQ ID NO:14)

DOM10-275-101

(SEQ ID NO:2468 in U.S. Ser. No. 12/152,903 & 12/397,826), (SEQ ID NO:15)

Codon-optimised DOM10-53-474 variants:—

Variant 1

(SEQ ID NO: 2470 in U.S. Ser. No. 12/152,903 & 12/397,826), (SEQ ID NO:3)

Variant 2

(SEQ ID NO: 2471 in U.S. Ser. No. 12/152,903 & 12/397,826), (SEQ ID NO:4)

Variant 3

(SEQ ID NO: 2472 in U.S. Ser. No. 12/152,903 & 12/397,826), (SEQ ID NO:5)

Codon-optimised DOM10-275-78 variant:—(SEQ ID NO: 2473 in U.S. Ser. No. 12/152,903 & 12/397,826), (SEQ ID NO:16)

1.-9. (canceled)

10. A method of inhibiting IL-13 (R130Q variant)-stimulated cell proliferation in a patient, the method comprising administering to the patient a therapeutically effective amount of ligand comprising DOM10-53-474 (SEQ ID NO:1).

11. A method for treating R130Q IL-13 variant-mediated disease or condition in a patient, where in the disease or condition is selected from the group consisting of allergic disease, bronchial hyperresponsiveness, Th2-type immune response, and asthma, the method comprising administering to the patient a therapeutically effective amount of ligand comprising DOM10-53-474 (SEQ ID NO:1).

12.-14. (canceled)

15. A nucleic acid comprising a codon-optimised sequence that encodes DOM10-53-474, optionally wherein the sequence is codon-optimised for expression in E coli or Pichia pastoris.

16. The nucleic acid according to claim 15, wherein the codon-optimised sequence is selected from SEQ ID NO: 3, SEQ ID NO: 4 and SEQ ID NO: 5.

17.-21. (canceled)

22. A ligand that has binding specificity for IL-13, comprising an immunoglobulin single variable domain with binding specificity for IL-13, wherein said immunoglobulin single variable domain with binding specificity for IL-13 inhibits binding of an anti-IL-13 domain antibody (dAb) selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10) to IL-13.

23. The ligand of claim 22, wherein said immunoglobulin single variable domain with binding specificity for IL-13 comprises an amino acid sequence that has at least about 75% amino acid sequence identity with the amino acid sequence of a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10).

24. The ligand of claim 22, wherein said immunoglobulin single variable domain with binding specificity for IL-13 has the epitopic specificity of a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10).

25. A ligand comprising an immunoglobulin single variable domain that binds IL-13, said immunoglobulin single variable domain selected from the group consisting of:
an immunoglobulin single variable domain wherein the amino acid sequence of said immunoglobulin single variable domain differs from the amino acid sequence of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10) at no more than 25 amino acid positions and has a CDR1 sequence that has at least 50% identity to the CDR1 sequence of a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10);
an immunoglobulin single variable domain wherein the amino acid sequence of said immunoglobulin single variable domain differs from the amino acid sequence of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10) at no more than 25 amino acid positions and has a CDR2 sequence that has at least 50% identity to the CDR2 sequence of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10); and
an immunoglobulin single variable domain wherein the amino acid sequence of said immunoglobulin single variable domain differs from the amino acid sequence DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10) at no more than 25 amino acid positions and has a CDR3 sequence that has at least 50% identity to the CDR3 sequence of a dAb selected from the group consisting of DOM10-275-78 (SEQ ID NO:6), DOM10-275-94 (SEQ ID NO:7), DOM10-275-99 (SEQ ID NO:8), DOM10-275-100 (SEQ ID NO:9) and DOM10-275-101 (SEQ ID NO:10).

26.-27. (canceled)

28. The ligand of claim 22, wherein the ligand is in an IgG-like format.

29. The ligand of claim 22 for therapy or diagnosis.

30. A ligand of claim 22 for treating, suppressing or preventing an allergic disease.

31. (canceled)

32. A ligand of claim 22 for treating, suppressing or preventing a Th2-type immune response.

33. (canceled)

34. A ligand of claim 22 for treating, suppressing or preventing asthma.

35. (canceled)

36. A ligand of claim 22 for treating, suppressing or preventing cancer.

37. (canceled)

38. A pharmaceutical composition comprising a ligand of claim 22 and a physiologically acceptable carrier.

39. An isolated or recombinant nucleic acid encoding a ligand of claim 22.

40. A vector comprising the recombinant nucleic acid of claim 39.

41. A host cell comprising the recombinant nucleic acid of claim 39.

42. A method of inhibiting proliferation of peripheral blood mononuclear cells (PBMC) in an allergen-sensitized subject, comprising administering to the subject a pharmaceutical composition comprising a ligand of claim 22.

43. A method of inhibiting proliferation of B cells in a subject, comprising administering to the subject a pharmaceutical composition comprising a ligand of claim 22.

44. (canceled)
